(19) United States
(12) Patent Application Publication
(10) Pub. No.: US 2003/0232102 A1
(43) Pub. Date: Dec. 18, 2003

(54) Total glycosides of paeony, method to prepare the same and uses thereof

(76) Inventor: Xinxian Zhao, Guangdong (CN)

Correspondence Address: Albert Wai-Kit Chan, World Plaza, Suite 604, 141-07 20th Ave., Whitestone, NY, 11357, US

(21) Appl. No.: 10/125,320

(22) Filed: Apr. 17, 2002

Related U.S. Application Data
(60) Provisional application No. 60/284,450, filed on Apr. 18, 2001.

(51) Int. Cl. A61K 35/78

(52) U.S. Cl. 424/778

(57) ABSTRACT

This invention provides a composition comprising Paeoniflorin, Albiflorin, Oxypaeoniflorin, and Benzoylpaeomiflorin. This invention also provides a pharmaceutical composition comprising an effective amount of Paeoniflorin, Albiflorin, Wxypaeoniflorin, and Benzoylpaeomiflorin and a pharmaceutically acceptable carrier. Finally, this invention provides a method to make the above composition.

This application claims the benefit of Provisional Patent Application No. 60/284,450, Filed on Apr. 18, 2001, the content of which is incorporated here into this application.

### BACKGROUND OF THE INVENTION

Rheumatoid Arthritis (RA) is an autoimmune disease that afflicts approximately 3% of the population, approximately 2.1 million Americans, mostly women. Diseases like RA that affect the entire human body are referred to as systemic illnesses. RA is characterized by inflammation of synovial tissues, joint pain, swelling and stiffness leading to varying degrees of joint destruction. RA is an extremely disabling disease that carries a high rate of immobility. The direct and indirect costs of RA on the U.S. economy reached $65 billion in 1992.

RA patients are treated initially with “first-line” agents: nonsteroidal anti-inflammatory drugs (NSAID) and corticosteroids, such as aspirin and cortisone, that act primarily to relieve the symptoms.

The majority of RA patients receive “second-line” medicines, the disease-modifying agents: Immunosuppressive medicines like methotrexate; Gold compounds; Hydroxychloroquine, drugs originally for malaria; Salfassalazine, drugs originally for ulcerative colitis; penicillamine; and so on. In a meta-analysis of 66 placebo-controlled trials comparing a variety of such second-line agents, no differences were found in short-term clinical efficacy for any of these agents, except oral gold (which was relatively less effective).

Among the second-line drugs, methotrexate (MTX) is said to have a more rapid onset of action. It appears to be more beneficial and has a relatively good safety profile. On the other hand, reports suggest that MTX should be generally reserved for patients with very aggressive disease, or those with serious complications of rheumatoid inflammation, and it should be used in low doses, usually in combination with anti-inflammatory agents to counter its serious side effects, which include: depression of bone marrow function, anemia, and/or a low white blood cell/platelet count, which can increase the risk of infection and bleeding. It can also lead to liver cirrhosis and allergic reactions in the lung.

There are many medicines for the treatment of RA, and reports tout new approaches, including monoclonal antibody therapy, tumor necrosis factor, etc., most of which are still in preclinical stages. It was known that most medication is high in side effects in comparison with their effectiveness, especially as long-term remedies.

### DETAILED DESCRIPTION OF THE FIGURES

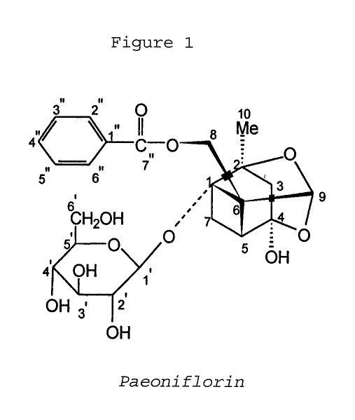

FIG. 1 Structural formula of Paeoniflorin

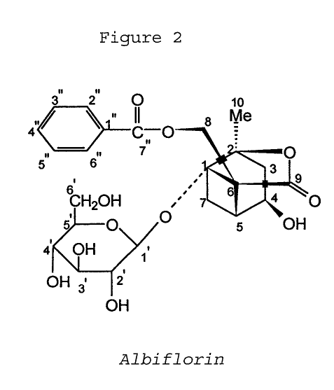

FIG. 2 Structural formula of Albiflorin

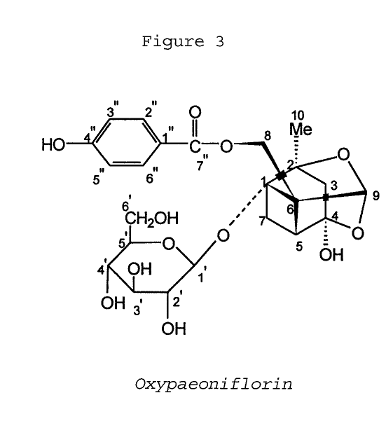

FIG. 3 Structural formula of Oxypaeoniflorin

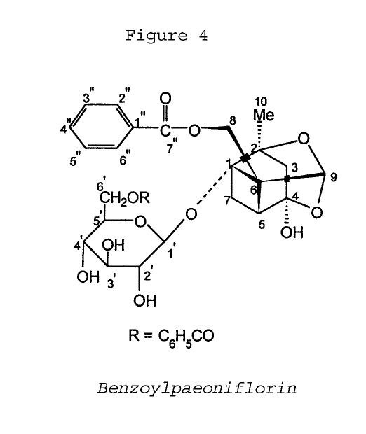

FIG. 4 Structural formula of Benzoylpaeoniflorin

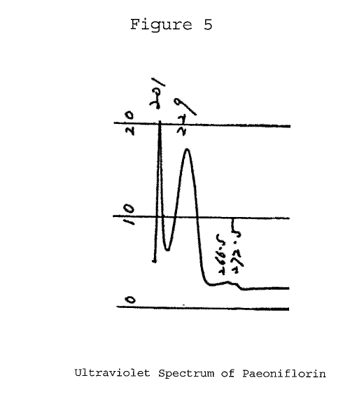

FIG. 5 Ultraviolet Spectrum V (PN3 0.000697 g/50 ml) of Paeoniflorin

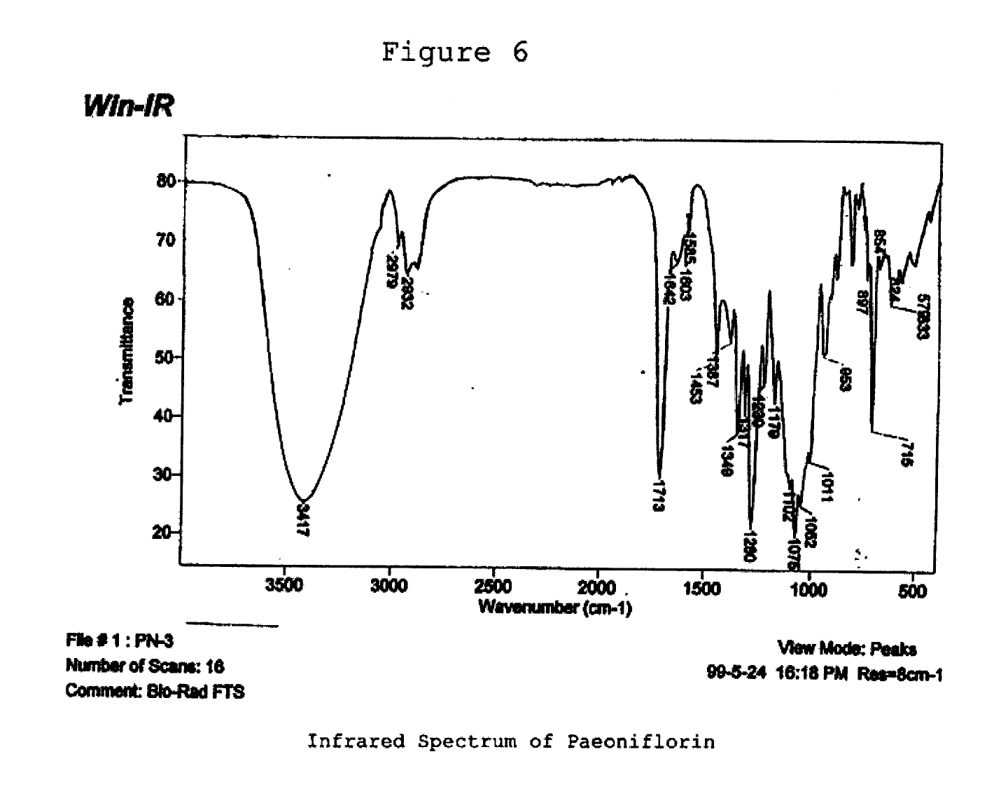

FIG. 6 Infrared Spectrum of Paeoniflorin

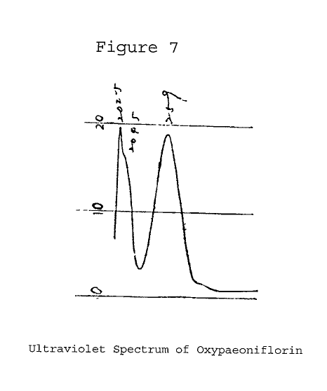

FIG. 7 Ultraviolet Spectrum (PN3 0.000707 g/50 ml) of Oxypaeoniflorin

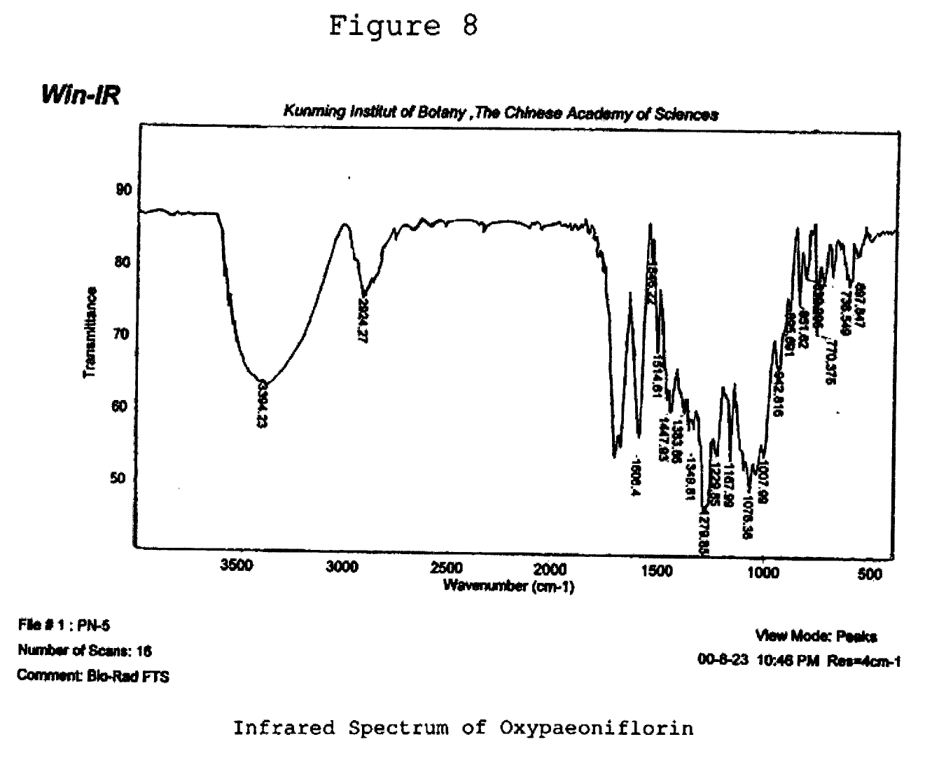

FIG. 8 Infrared Spectrum of Oxypaeoniflorin

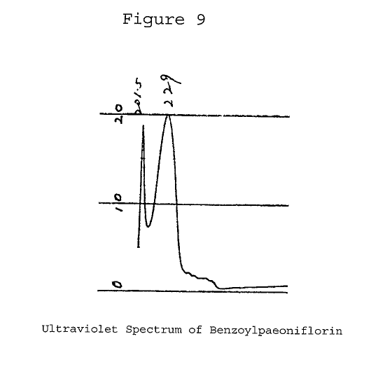

FIG. 9 Ultraviolet Spectrum (PN3 0.000502 g/50 ml) of Benzoylpaeoniflorin

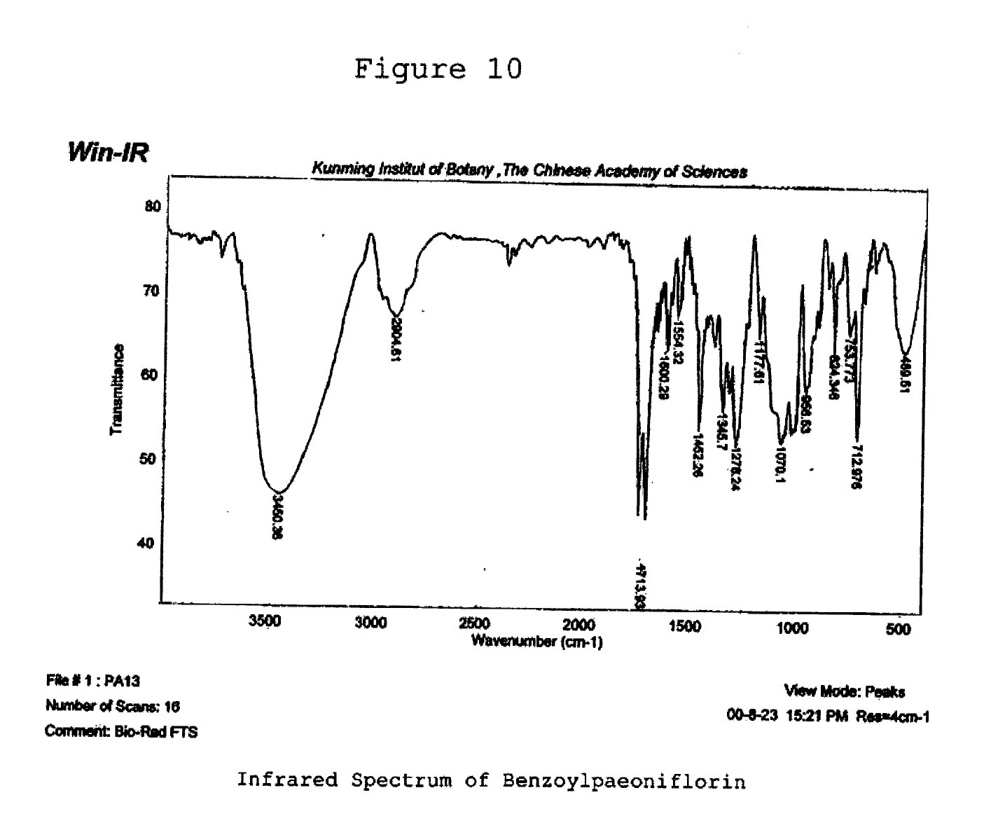

FIG. 10 Infrared Spectrum of Benzoylpaeoniflorin

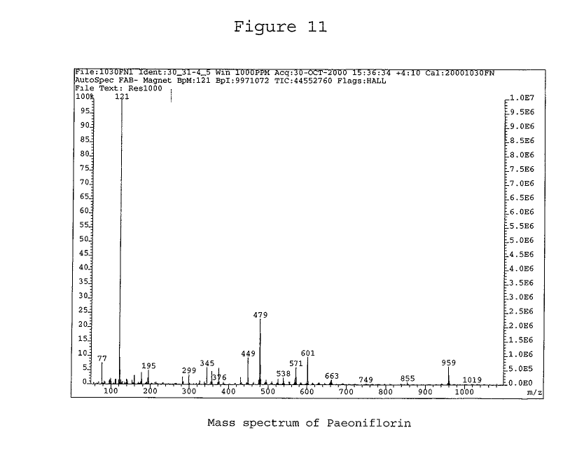

FIG. 11 Mass spectrum of Paeoniflorin

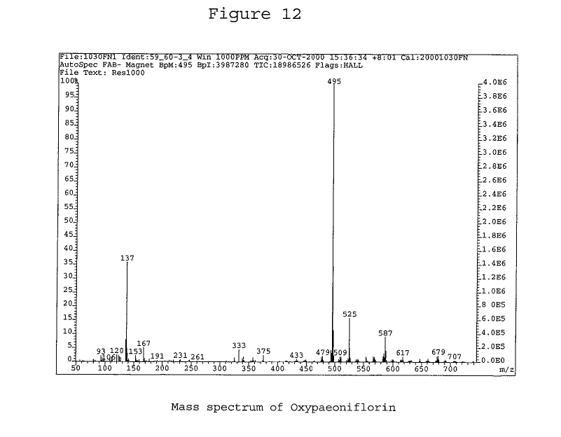

FIG. 12 Mass spectrum of Oxypaeoniflorin

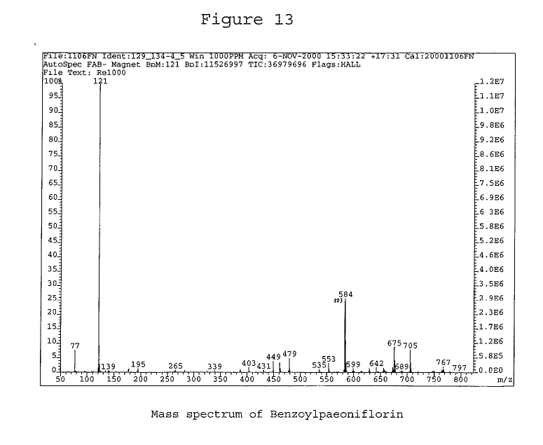

FIG. 13 Mass spectrum of Benzoylpaeoniflorin

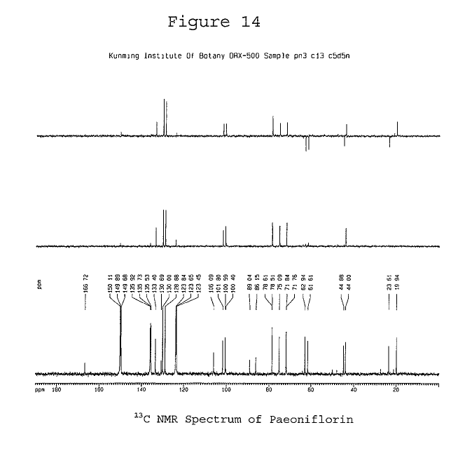

FIG. 14 13C NMR Spectrum of Paeoniflorin

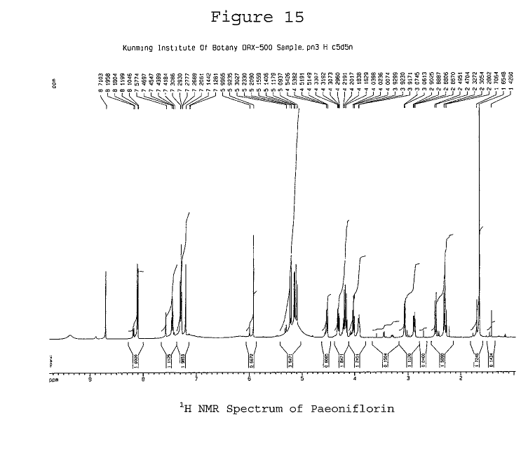

FIG. 15 1H NMR Spectrum of Paeoniflorin

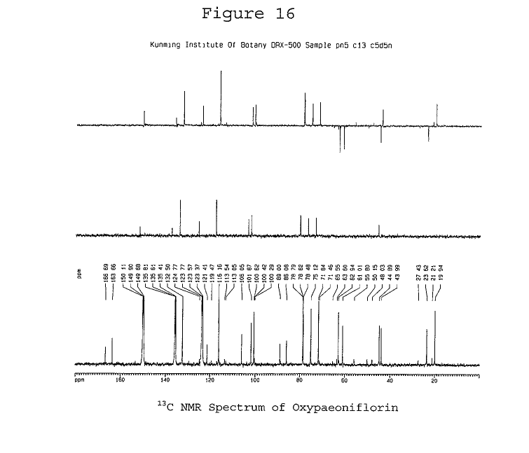

FIG. 16 13C NMR Spectrum of Oxypaeoniflorin

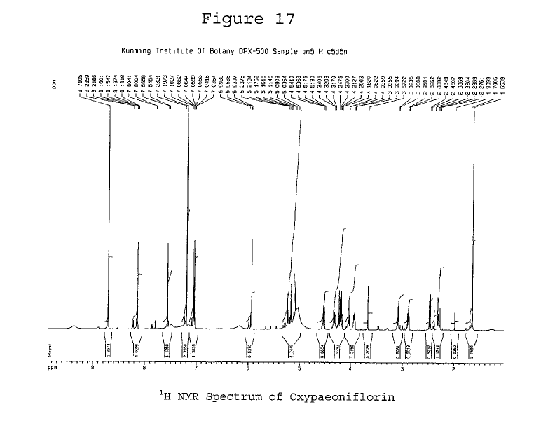

FIG. 17 1H NMR Spectrum of Oxypaeoniflorin

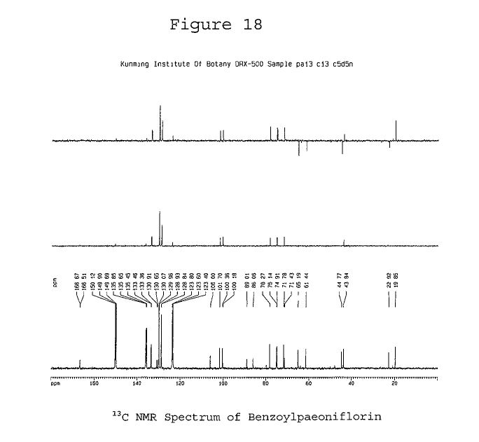

FIG. 18 13C NMR Spectrum of Benzoylpaeoniflorin

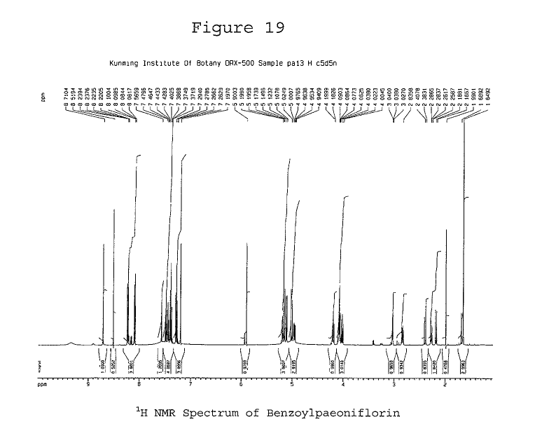

FIG. 19 1H NMR Spectrum of Benzoylpaeoniflorin

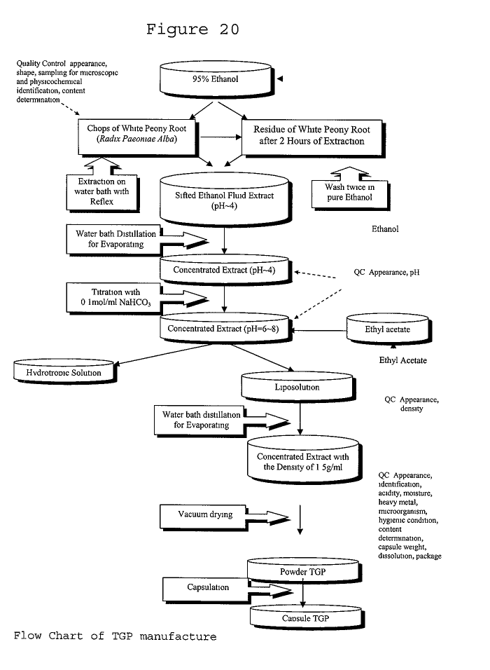

FIG. 20 Flow Chart of manufacture

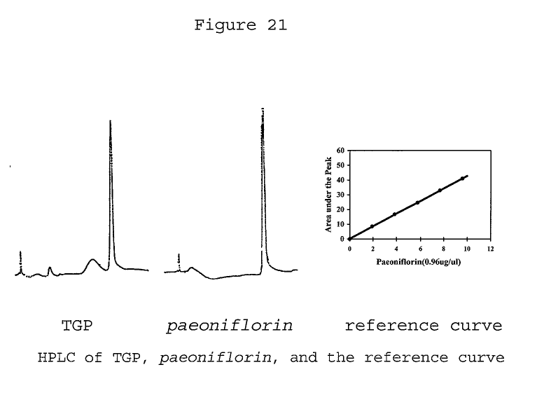

FIG. 21 HPLC of TGP, paeoniflorin and the refernce curve (paeoniflorin)

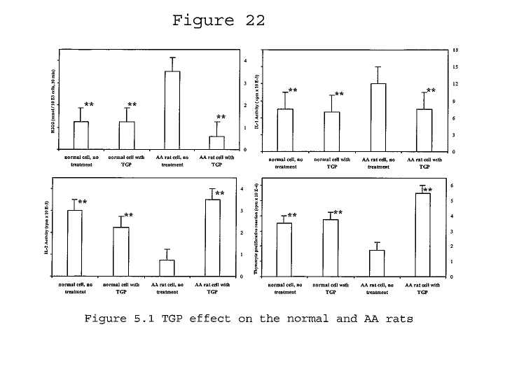

FIG. 22 TGP effect on normal and AA rats: H2O2 and IL-1 generated by M φ in abdominal cavity, Con A proliferative reaction of thymocyte, and the Con A induced IL-2 creation of splenocyte. AA rats were administered TGP (50 mg/kg d) from d7 to d17. Rats were executed on d17 and examined of the indexes. Mean±SD, n=5, **P<0.01 in compare with AA, no treatment group

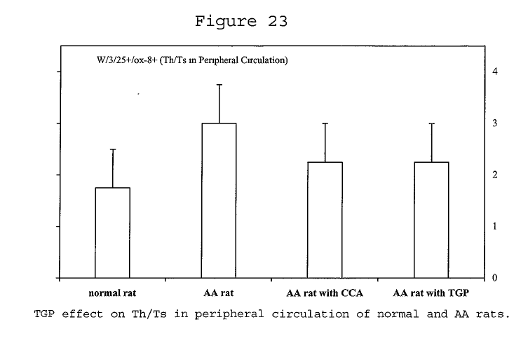

FIG. 23 TGP effect on Th/Ts in peripheral circulation of normal and AA rats: AA rats were administered TGP (50 mg/kg d) from d18 to d25. Rats were executed on d25 and examined of the indexes. Mean±SD, n=6, **P<0.01, in compare with AA, no treatment group.

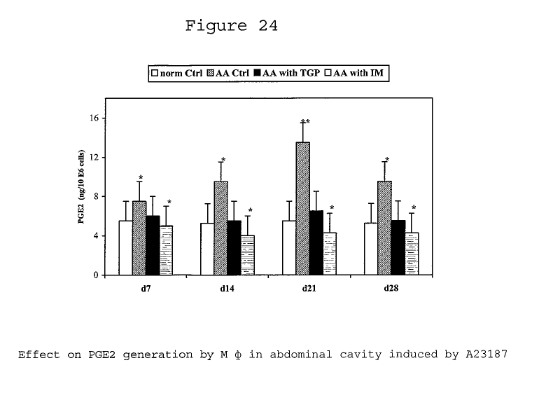

FIG. 24 TGP effect on PGE2 generation by macrophage in abdominal cavity induced by A231187: AA rats were administered TGP (50 mg/kg d) or IM (2 mg/kg d) from do. Rats were executed on d7, d14, d21, and d28 for the exam of PGE2. Mean±SD, n=4, *P<0.05, **P<0.01, in compare with normal control group.

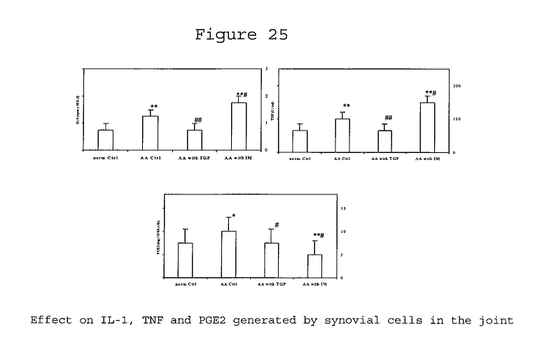

FIG. 25 TGP effect on IL-1, TNF and PGE2 generated by synovial cells in joints: AA rats were administered TGP (50 mg/kg d) or IM (2 mg/kg d) from d12. Rats were executed on d22 for the exam of above indexes.

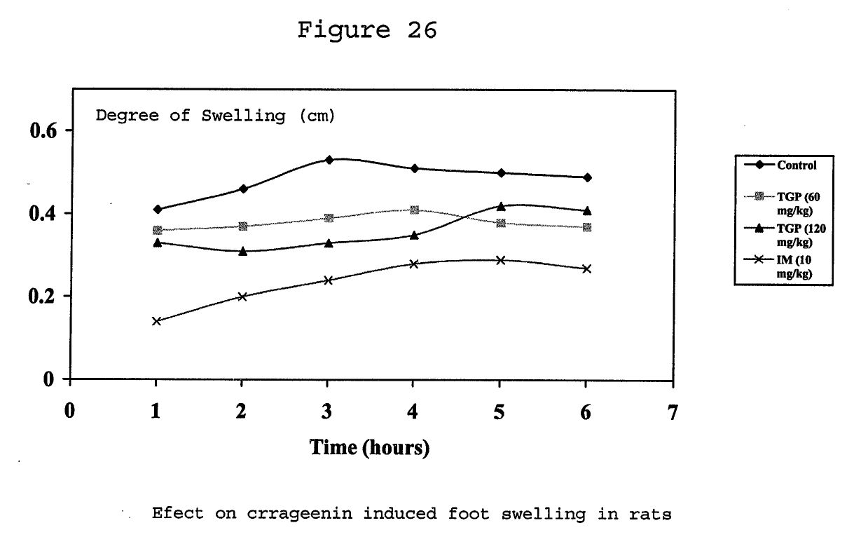

FIG. 26 TGP effect on crrageenin induced foot swelling in rats

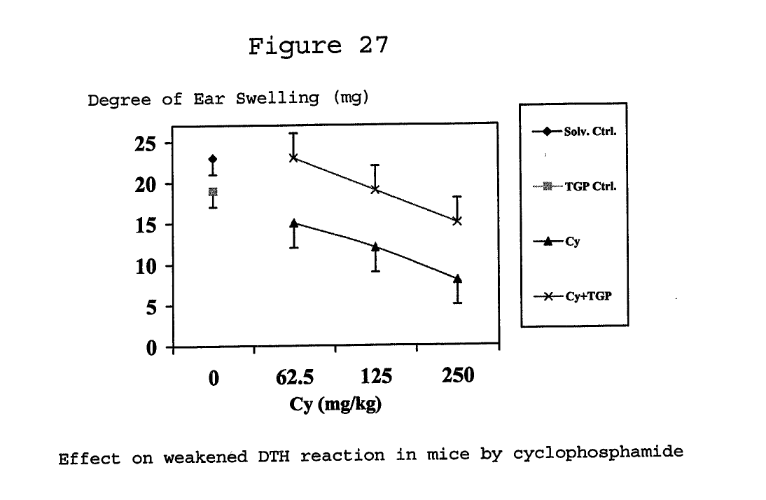

FIG. 27 TGP effect on weakened DTH reaction in mice by cyclophosphamide: Mean±SD, n=7; *P<0.05, **P<0.01 in compare with control group

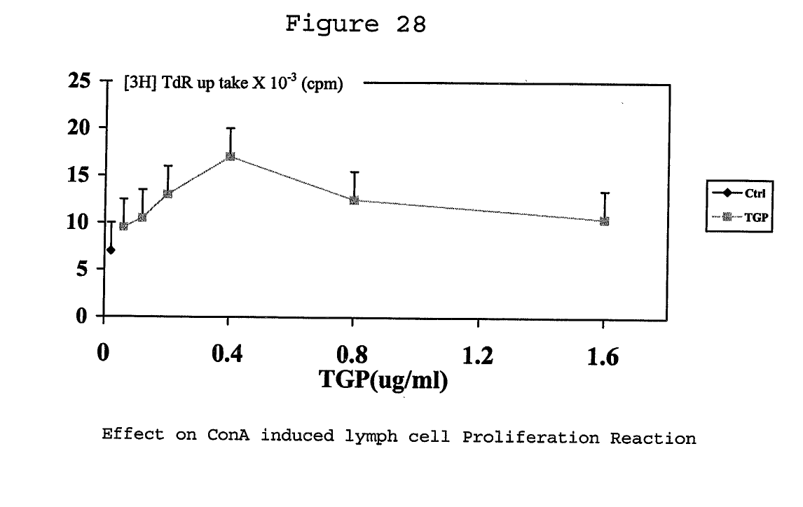

FIG. 28 TGP effect on ConA induced lymph cell Poliferation Reaction: Mean±SD, n=8; *P<0.05 in compare with control group (con A 3 μg/ml)

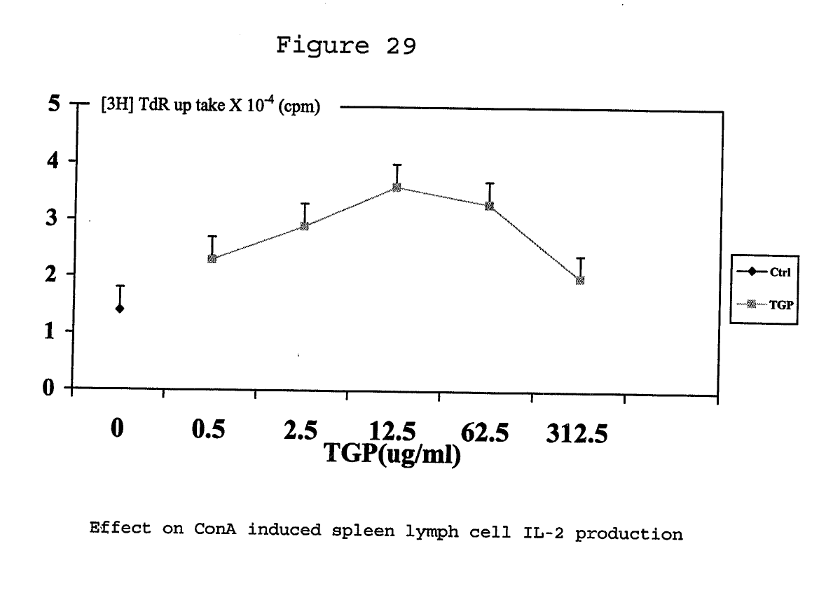

FIG. 29 TGP effect on ConA induced spleen lymph cell IL-2 production: Mean±SD, n=5; logarithmic diagram, *P<0.05 in compare with control group (con A 3 μg/ml)

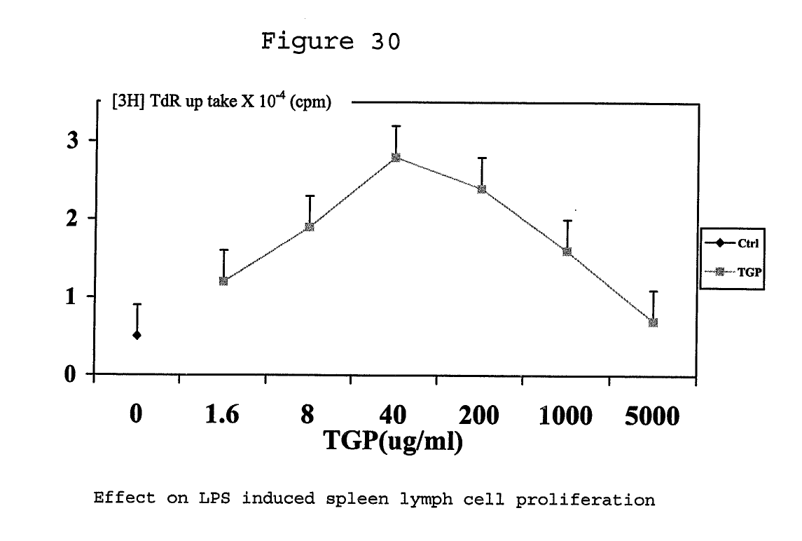

FIG. 30 TGP effect on LPS induced spleen lymph cell proliferation: Mean±SD, n=8; logarithmic diagram, *P<0.01 in compare with control group (LPS 6 μg/ml)

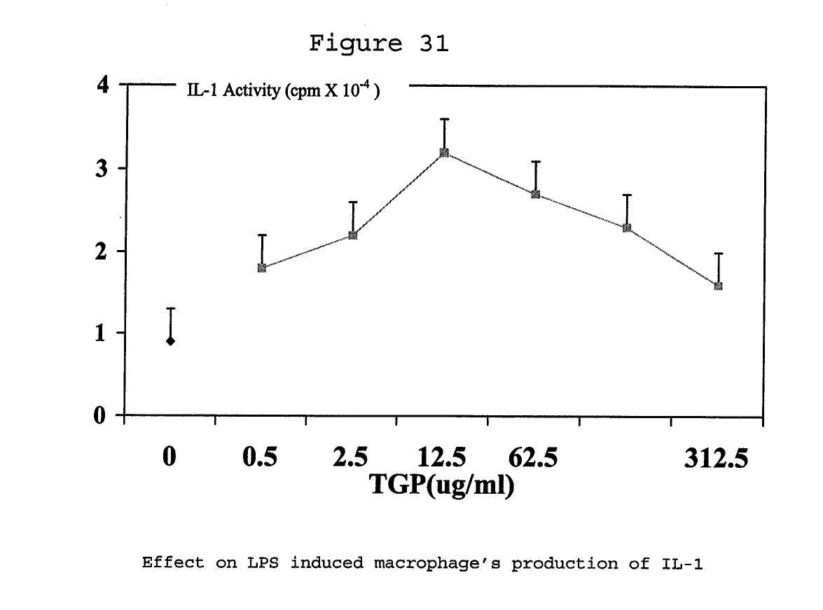

FIG. 31 TGP effect on LPS induced macrophage's production of IL-1: Mean±SD, n=4; logarithmic diagram, **P<0.01 in compare with control group (LPS 6 μg/ml)

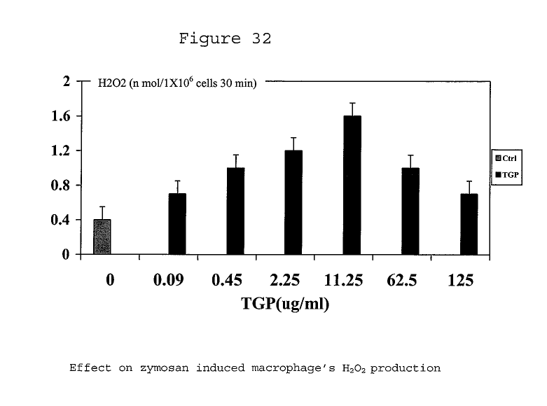

FIG. 32 TGP effect on zymosan induced macrophage's H2O2 production: Mean±SD, n=5; logarithmic diagram, *P<0.05, **P<0.01, in compare with control group

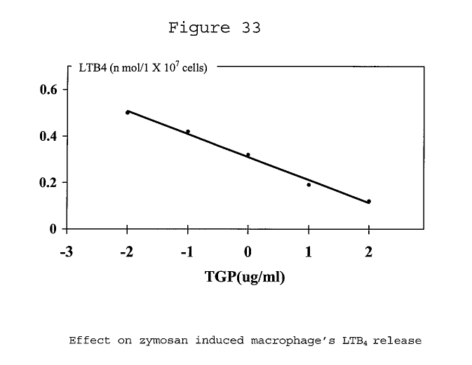

FIG. 33 TGP effect on zymosan induced macrophage's LTB4 release

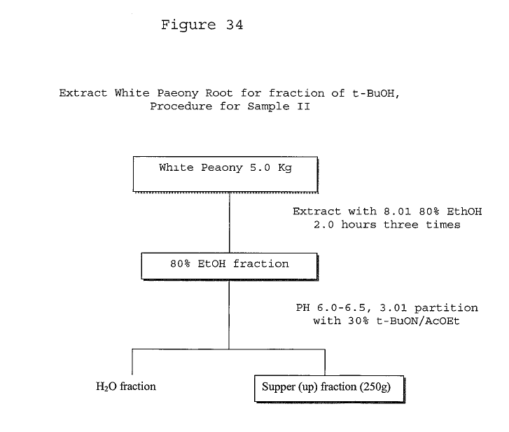

FIG. 34 Flow chart for extract White Paeony Root for fraction of t-BuOH, procedure for Sample II

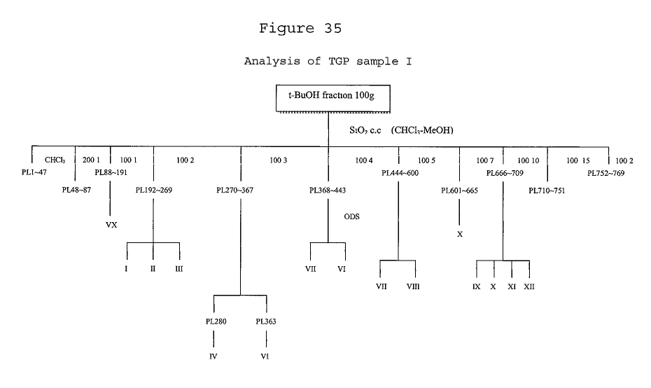

FIG. 35 Analysis of TGP Sample I

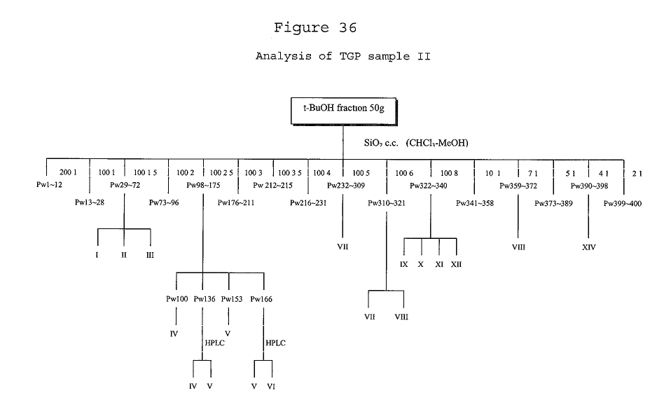

FIG. 36 Analysis of TGP Sample II

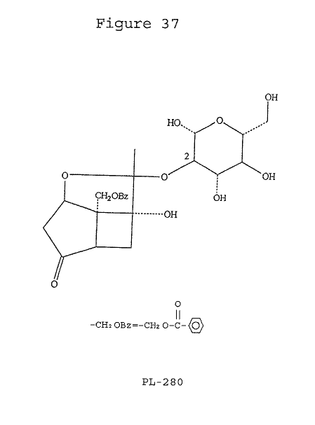

FIG. 37 Structure of PL 280

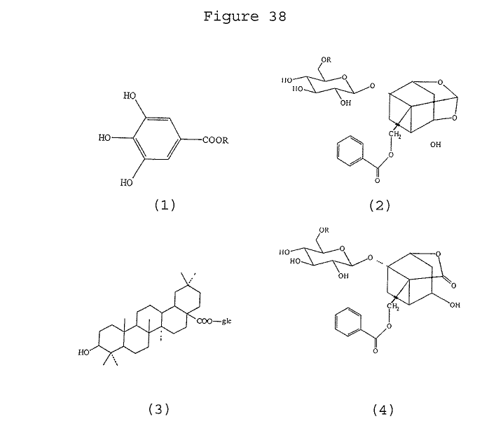

FIG. 38 Structures of compounds isolated from TGP

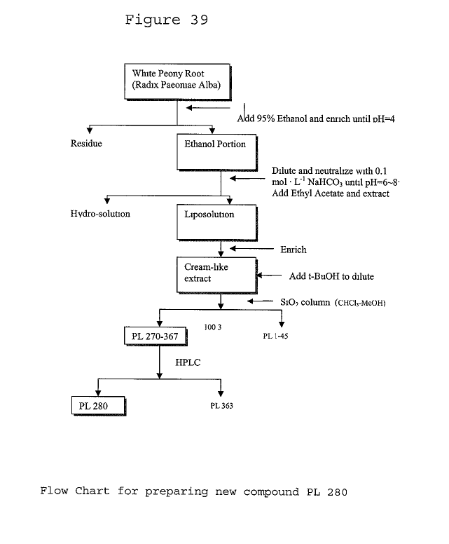

FIG. 39 Flow chart for preparing new compound PL 280

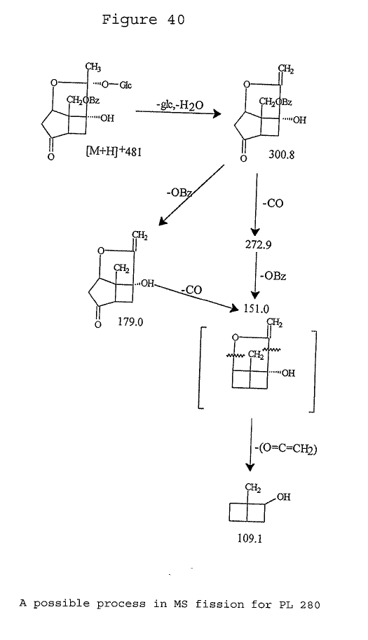

FIG. 40A process in MS fission for PL 280

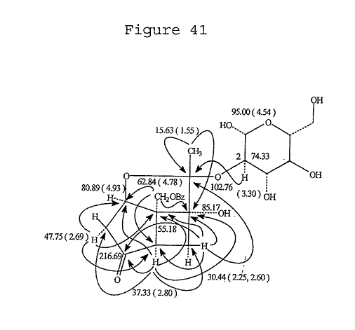

FIG. 41 HMBC spectrum of PL 280

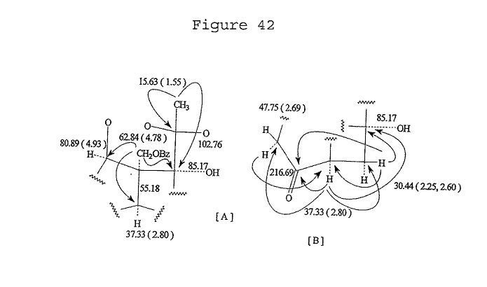

FIG. 42 Section [A] and [B] of PL 280, analyzed with HMBC spectrum

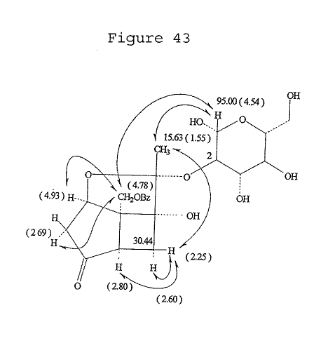

FIG. 43 NOESY spectrum: NOE correlations of PL 280

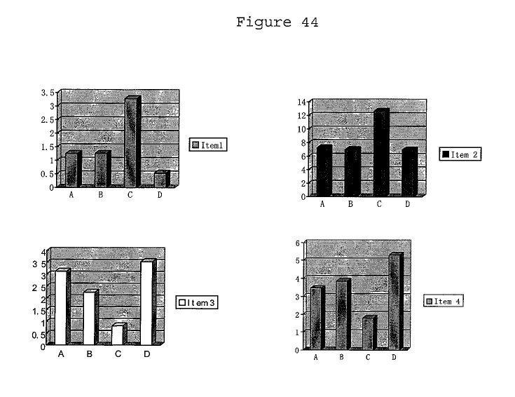

FIG. 44 TGP interferes the amount of H2O2, IL-1, and PGE2 produced by abdominal cavity macrophage: Item 1-H2O2 (nmol/104, 30 min), Item 2-IL-1 Activity (cpm×10-3), Item 3-IL-2 Activity (cpm×10-3), Item 4-Proliferation (cpm, 10-4)

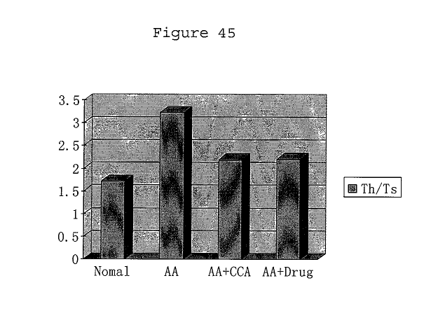

FIG. 45 TGP interferes sub-group T cell in peripheral circulation

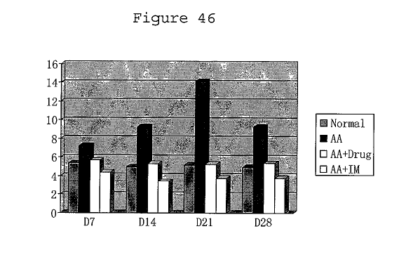

FIG. 46 TGP interferes kinemics in abdominal cavity macrophage release PGE2

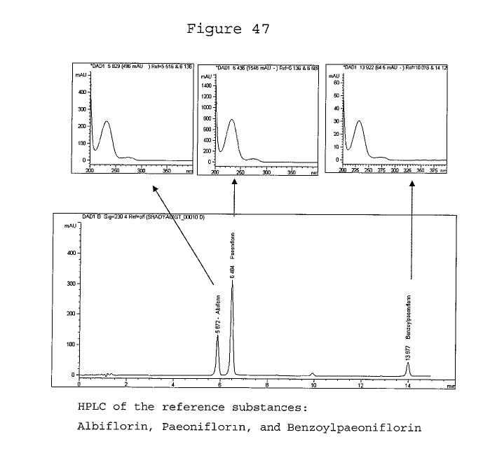

FIG. 47 HPLC of the reference substances: Albiflorin, Paeoniflorin, and Benzoylpaeoniflorin

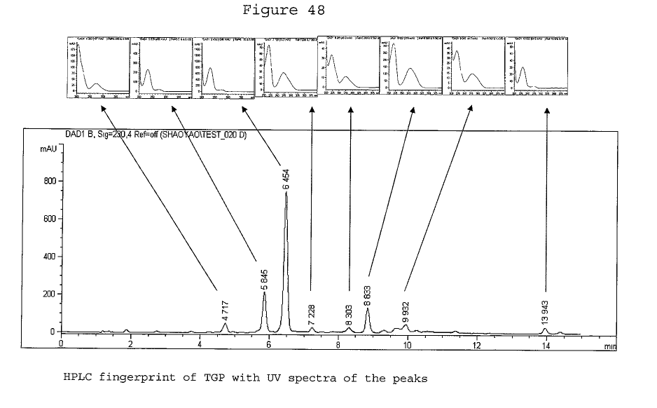

FIG. 48 HPLC fingerprint of TGP with TV spectrum of the peaks: (Peak 2: Albiflorin, peak 3: Paeoniflorin, peak 8: Benzoylpaeoniflorin)

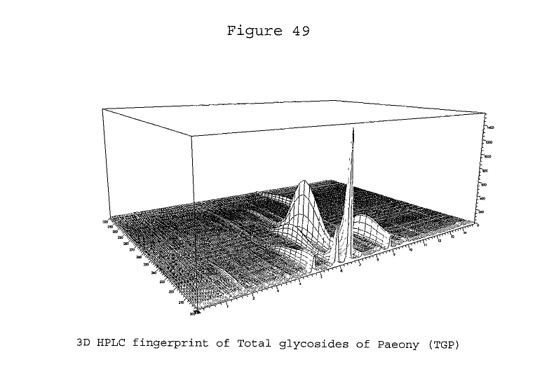

FIG. 49 3D HPLC fingerprint of Total glycosides of Paeony (TGP)

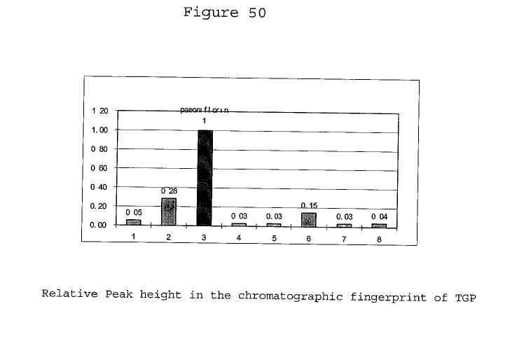

FIG. 50 Relative Peak height in the chromatographic fingerprint of TGP

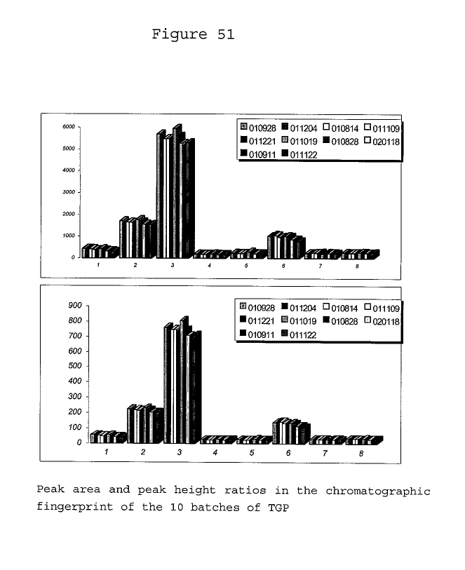

FIG. 51 Peak area and peak height ratios in the chromatographic fingerprint of the 10 batches of TGP

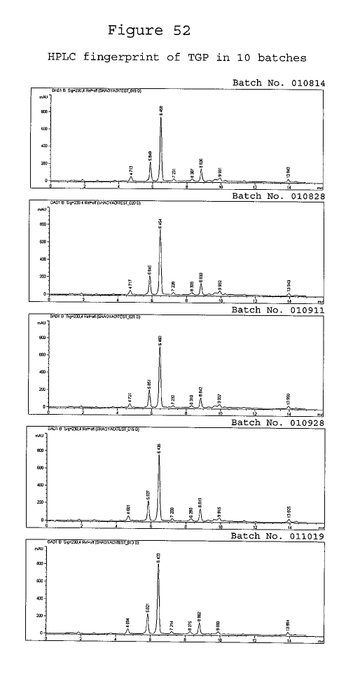

FIG. 52 HPLC fingerprint of TGP in 10 batches

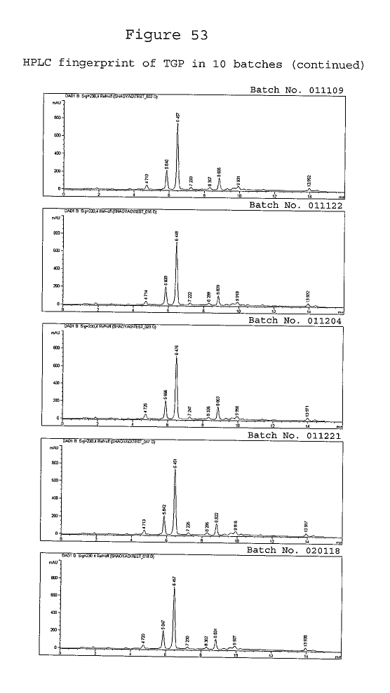

FIG. 53 HPLC fingerprint of TGP in 10 batches

### DETAILED DESCRIPTION OF THE INVENTION

The present invention provides a composition comprising Paeoniflorin, Albiflorin, Oxypaeoniflorin, and Benzoylpaeomiflorin. This invention also provides the composition above, wherein the proportion of Paeoniflorin, Albiflorin, Oxypaeoniflorin, and Benzoylpaeomiflorin is 85-90%. In another embodiment, the proportion of Paeoniflorin is no less than 35%. The invention further provides the composition above, derived from an extract of white peony. In a further embodiment, the composition is derived from the root of white peony.

The invention also provides the pharmaceutical composition comprising an effective amount of Paeoniflorin, Albiflorin, Oxypaeoniflorin, and Benzoylpaeomiflorin and a pharmaceutically acceptable carrier.

The invention provides the pharmaceutical composition above, wherein the proportion of Paeoniflorin, Albiflorin, Oxypaeoniflorin, and Benzoylpaeomiflorin is 85-90%. The invention further provides the pharmaceutical composition above, wherein the proportion of Paeoniflorin is no less than 35%. Furthermore, the invention provide a formulation comprising the pharmaceutical composition above, wherein the formulation is a pill, capsule, granule, tablet, suspension, injection, syrup, or tincture.

This invention further provides a method for producing the composition above comprising steps of: (a) obtaining appropriate herbal materials; (b) chopping the obtained herbal materials into small pieces; (c) immersing the herbal materials into an organic solvent for extraction; (d) separating the extracted materials into residue and solution and repeating step c at appropriate times for the residue; (e) combining solutions from the extractions; (f) concentrating the solution from step e; (g) diluting the solution from step f to approximately 6.0 in pH; (h) extracting solutions from step g in appropriate solutions to obtain lipo-solutions; (i) concentrating the combined lipo-solution from step h; and (j) vacuum drying the extract from step i to obtain the composition above. The invention also provides the method above, wherein in step c and d, the chopped herbs are extracted three times in 95% alcohol. The invention further provide the method above, wherein, in step h, the extracting is ethyl acetate. Furthermore, the invention provides the method above, wherein the herb is white peony. In a further embodiment, it is the root of the white peony.

In addition, this invention provides the composition produced by the above method. The invention also provides the composition above, wherein, it comprises Paeoniflorin. The invention further provides the composition above, e wherein, it further comprising Albiflorin, Oxypaeoniflorin, and Benzoylpaeomiflorin.

This invention also provides a pharmaceutical composition described above and a pharmaceutically acceptable carrier.

This invention further provides a method for treating arthritis in a subject comprising administering to the subject the pharmaceutical composition above. In an embodiment, the subject is human. The invention also provides a method for alleviating clinical symptoms in a subject suffering from arthritis comprising administering to the subject the pharmaceutical composition above. Furthermore, this invention provides the method above, wherein the arthritis is a rheumatic arthritis.

Furthermore, this invention provides a method for adjusting immunity in a subject comprising administering to the subject the pharmaceutical composition above.

This invention provides a compound with the structure set forth in FIG. 37 and derivatives of the compound above. This invention also provides a composition and a pharmaceutical composition comprising the compound above, and a pharmaceutically acceptable carrier. This invention further provides a method for treating inflammatory conditions or immune disorders in a subject comprising administering to the subject the pharmaceutical composition above or the composition above. Furthermore, this invention provides a method for producing the compound above as set forth in FIG. 39.

This invention further provides the fingerprinting of Total Glycosides of Paeony as set forth in FIG. 48. This invention also provides Total Glycosides of Paeony as characterized by at least 4 of the 8 peaks recited, wherein if the retention time of paeoniflorin is 1, the corresponding value for relative retention times are: 0.73 for peak 1, 0.91 for peak 2, 1 for peak 3, 1.12 for peak 4, 1.29 for peak 5, 1.37 for peak 6, 1.54 for peak 7, and 2.16 for peak 8. The areas render these peaks can be ±15%. In another embodiment, the areas are ±10%. This invention further provides Total Glycosides of the above Paeony, characterized by at least 5 peaks of the 8 peaks recited above. Furthermore, this invention provides the above Total Glycosides of Paeony, characterized by at least 6 peaks of the 8 peaks recited above. Finally, this invention provides Total Glycosides of Paeony above, characterized by at least 7 peaks of the 8 peaks recited above.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Total Glycosides of Paeony is a botanical drug, an extract from the dried root of White Peony (Paeonia Lactiflora Pall.). It can be used for treating Rheumatoid Arthritis. The data from a large-scaled clinical study conducted in China suggested that TGP introduces substantial benefits to patients. It shares the same therapeutic efficacy as MTX, a classic effective remedy for RA, while showing much fewer negative influences on the human body.

White peony is commonly called peony, or Chinese peony, a fragrant white-flowered species that is one of the most popular herbaceous peonies throughout the world. Its dried root is one of the most important element herbs in Traditional Chinese Medicine (TCM), which has been medically used for about one thousand years in recorded history. In China, the description of its medicinal effects can be found in nearly every traditional medical book in successive dynasties.

TGP originated from the most developed prescriptions in TCM compiled with remedies from people with a long history of civilization. The white peony is commonly used in Asia. It is so popular in herb therapies that it has been described both in Chinese and Japanese pharmacopoeia. In recent years, people came to notice that its traditionally described functions were mostly focused on the human immune system. It has been developed for its immune interference characteristics.

One of the main ingredients, Paeoniflorin, was separated and purified from peonies in early 60's and its pharmacological functions of sedation, pain relief and anti-inflammation were discovered. The conclusion can also be drawn from serials of pre-clinical studies: in the experiments, it was observed that TGP which sustains its chief ingredient as Paeoniflorin and its derivates may activate the phagocytic function of macrophagocyte and inhibit swelling in the ankle joint of rats. It can moderate the immunologic abnormalities in rat adjuvant arthritis and regulate the extra higher or lower conditions of humoral and cellular immunity induced by cyclophosphamide to become normal. Its two-way regulation effect on immune function is dosage dependent.

Total Glycosides of Paeony has been established as a safe and effective remedy in providing symptomatic relief and systematic improvement in chronic inflammation of RA.

Raw Materials

Total Glycosides of Paeony (TGP) is the extract from dry white peony root (Radic Paeoniae Alba).

White peony (Paeonia Lactiflora Pall. [P. Albiflora Pall]) is commonly called peony, white peony or Chinese peony which is one of the most popular herbaceous peonies.

The cultivated species have been the common source for drugs. There are three large production arias of white peony root (Radix Paeoniae Alba) that have long been established in China. They are Bozhou, Hangzhou and Heze, such areas are centers for the growth, harvest, crude drug preparation and product distribution. People who work in these large-scaled farms and markets are well skilled and experienced in the pertinent procedures, since the production of a good and stable drug product has a long practice and business history. White peony root is one of the most commonly used crude botanical drugs in TCM, and there is a large demand on it every year. The white peony root for TGP product is also called “Bo” white peony since it is from Bozhou, China.

The White Peony (Paeonia Lactiflora Pall.)

Paeonia lactiflora is a herbaceous, deciduous perennial, which grows to 2-3 feet in height. It has straight, upright stems, large and dissected leaves, which are medium to dark green. It features two ternate or occasionally pinnate leaves with-many oval to linear ones, and entire or lobed leaflets. It has large double flowers with ruffled petals thickly and neatly arranged. From early to mid-summer, they open with white or rose-pink flush. In addition to its beautiful flowers, it is valued for its tuberous roots, which are shaped like a spindle or column, and are deep brown or black.

Growing in meadows, scrub rocky hillside or woodland, it is mostly native to Siberia, Mongolia, northern parts of Manchuria, and Tibet. In China, the wild species are distributed over Dongbei, the Huabei plains, Shanxi, and Gansu. For more than 800 years, the plant was cultivated not only for its cheerful flowers but also as one of the basic drug materials of TCM.

Planting of White Peony

Planted in deep, fertile, humus-rich soil with good drainage, the plant prefers full sun and can tolerates light shade, cold weather and most soils. Seed sowing and root division can both be used for propagation. It takes 3-4 years of growth before it may harvested, and it is mostly a trouble-free plant. Fungi can be the main problem. Botrytis paeonia and Botrytis cinerea are important pests. Spider mite, aphid, grub and cutworm can be harmful insects.

The White Peony Root (Radix Paeoniae Alba)

After 3-4 years of growth, the plant is gathered in September or October. The planters dig out the whole plant from the earth, cut away stems grown above the ground, and clean dirt from the root. The root is washed in clear water and then boiled for 5-15 minutes until no hard core can be observed. The boiled root is easily peeled with a bamboo knife and chopped into slices. Dried completely in the sun, the slices (Radix Paeoniae Alba) are stored in a dry, cool and well-ventilated warehouse. Radix Paeoniae Alba is white with some wood-like slices, and it is relatively hard and heavy. The cross section of the slice appears grayish-white, sometimes with a hint of brown. A radiate grain can be seen in its xylem. It has no smell and tastes sour and slightly bitter.

Quality Control for White Peony Root

A standard for quality control of the crude drug materials has been established in compliance with Good Manufacturing Practice (GMP) in China. White peony roots are assessed for their quality and content of active ingredients. The white peony root should be dry, clean, free of contamination by dirt, mold, rot, and any other contamination. Release criteria: Chinese Pharmacopoeia Volume 1 1990 Edition, P 85 (Refer to relevant chapters for detailed information.)

Quality Control for Other Materials

Ethanol (95%), Ethyl acetate and NaHCO₃ are “industrial grade” purities. The water is unpolluted and purified living water.

Drug Substance

White Peony Root (Radix Paeoniae Alba) contains 1) Paeoniflorin, Oxypaeoniflorin, Benzoylpaeoniflorin, Albiflorin, and many other miner components. The above four glycosides share a similar chemical structure and are considered capable of transforming into one another under the appropriate conditions. They account for 85-90% of the total mass of TGP, and are the active ingredients. 2) Other glycosides, such as: paeoniflorigenone, daucosterol, lactoflorin etc., which exist in small amounts; 3) Carbohydrates and tanning matters; 4) Volatile oils: benzoic acid, paeonol etc.

The bulk drug TGP is obtained from Radix Paeoniae Alba through an effective two-step extraction with alcohol and ethyl acetate. After vacuum drying the extract fluid, the semi-product is made into a powder. When this powder is filled into capsules without excipient, the result is the final product. Each capsule contains 300 mg of powder TGP.

Manufacturing Process

The operating procedures and outcome ratios of manufacturing are described below:

Add 200 ml of 95% alcohol in 100 grams of white peony root. Heat the alcohol in a circulation reflex for 2 hours in water bath. The solution is then collected after cooling down and filtrating. The residue is washed twice in 95% alcohol and the alcohol saved to be mixed with the formal alcoholic solutions. After distilling the mixed alcoholic extract in a water bath, 15 ml of concentrated extract is finally obtained. The acidity is adjusted with dilute alkali until its PH=6.0. The balanced concentrated alcoholic extract is then extracted with 30 ml of ethyl ,:12 acetate three times (90 ml in total). The ester extract is distilled in a water bath until its density comes up to 1.5 g/ml, yielding 6.0 grams of cream extract (outcome rate: 4.5%). After vacuum drying in a drying cupboard (pressure: 15 mm Hg, temperature: 95-100° C.), 3.0 grams of TGP powder is extracted.

White peony root (dried) contains 65% carbohydrates, 3.5-5% of TGP and many other contents, including β-sitosterin, benzene carboxylic acid, tanning matters, volatile oils, etc. The technological procedure described above can extract most TGP effectively from natural materials in a relatively pure form.

There is about 10 grams of solid components in the alcoholic extract from 100 grams of white peony root (after the first step of extraction):

TABLE 1
TGP preparation: contents in step one

| | Benzene | | | | |
|---|---|---|---|---|---|
| | Carboxylic | | Tanning | | |
| TGP | Acid | Carbohydrates | Matters | Other | Total |
| 3.2 g | 4.0 g | 1.8 g | 0.4 g | 0.6 g | 10.0 |

After the neutralization reaction (0.1 mol/L NaHCO₃) and the extraction of ethyl acetate, 3.0 grams of solid material is released. That is the powder product TGP.

The TGP capsules are prepared by filling the TGP powder into capsules, 300 mg in each capsule without any excipient or subsidiary.

TABLE 2
TGF preparation: contents after step two

| TSP | | | |
|---|---|---|---|
| (the 4 active | | | |
| ingredient | | Tanning | |
| glycosides) | Carbohydrates | Matters | Total |
| 2.6 g | 0.2 g | 0.18 g | 3.0 g |

Qualitative and Quantitative Description

| | |
|---|---|
| Name of Bulk Drug: | Total Glycosides of Paeony (TGP) |
| Active Ingredients: | Paeoniflorin |
| | Oxypaeoniflorin |
| | Benzoylpaeoniflorin |
| | Albiflorin |
| Other components: | Carbohydrates: 8% in total mass |
| | Tanning matters: 6.5% in total mass |
| | Other glycosides: paeoniflorigenone, |
| | daucosterol, etc., trace |
| Appearance: | A light yellowish brown powder |
| Taste: | Slight bitter and sour |
| Acidity: | pH in 10% solution: 6.5 |
| Coefficient of Absorptinont: | E_{1cm}^{1%} (230) = 175 |
| Specific Rotation Power | [a] _{D}²⁰ − 13.6 (C = 5.2) |
| Solubility: | Hygroscopic powder; soluble in water, |
| | ethanol and ethyl acetate; Slightly |
| | soluble in diethyl ether and chloroform |
| Clinical Indication: | For treatment of Rheumatoid |
| | Arthritis |
| Manufacturer: | Sunjiu Medical & Pharmaceutical |
| | Co., Ltd. |
| | 1028 Beihuan Road |
| | Shengzhen, Guangdong 518029 |
| | P. R. China |

TABLE 3
Properties of the active ingredience (1)

| Name | Paeoniflorin | Albiflorin |
|---|---|---|
| Mol. | C₂₃H₂₈O₁₁ | C₂₃H₂₈O₁₁ |
| formula | | |
| Mol. | 480 | 480 |
| weight | | |
| Appear- | Amorphous white | Colorless hygroscopic |
| ance | powder | powder |
| Solution | Neutrality | Neutrality |
| acidity | | |
| UV (MeOH) | λ ₘₐₓ (log ε) | λ ₘₐₓ (log ε) 231 (4.05), |
| | 201 (4.10), | 267 (3.15), 274 (3.16), |
| | 229 (4.02), | 281 (3.08) nm |
| | 266.5 (2.44), | |
| | 272.5 (2.65) nm | |
| IR (KBr) | 3417 (broad, OH), | 3400 (broad, OH), |
| νₘₐₓ | 2932, 1713 (OBz), | 2904, 1760 (lactone) |
| | 1603 (phenyl) , | 1720 (Obz) , 1605, 1590 |
| | 1453, 1387, 1349, | (phenyl)/cm |
| | 1280, 1075, 953, | |
| | 715/cm | |
| Negative | 479 [M-H]⁻ (25), | 479 [M—H]⁻ (30), 121 |
| FAB-MS | 449 (10), 121 | (10), 121 [C₆H₅COO]⁻ |
| m/z (%) | [C₆H₅COO]⁻ (100), | (100), 77 [C₆H₅]⁻ (10) |
| | 77[C₆H₅]⁻ (8) | |

TABLE 4
Properties of the active ingredience (2)

| Name | Oxypaeoniflorin | Benzoylpaeoniflorin |
|---|---|---|
| Mol. | C₂₃H₂₈O₁₂ | C₃₀H₃₂O₁₂ |
| formula | | |
| Mol. weight | 496 | 584 |
| Appear-ance | Amorphous white | Amorphous white powder |
| | powder | |
| Solution | Neutrality | Neutrality |
| acidity | | |
| UV (MeOH) | λ ₘₐₓ (log ε) | λ ₘₐₓ (log ε) |
| | 202.5 (4.10), | 201.5 (4.31), 229 (4.43) |
| | 204.5 (4.06), | nm |
| | 259 (4.09) nm | |
| IR (KBr) | 3394 (broad, OH), | 3450 (broad, OH), 2904, |
| νₘₐₓ | 2924, 1721 (OBz), | 1713 (Obz), 1600, |
| | 1606, | 1554 (phenyl), 1452, |
| | 1514 (phenyl), | 1345, 1278, 1177, 1070, |
| | 1447, 1383, 1349, | 956, 712/cm |
| | 1279, 1167, 1076, | |
| | 942, 770/cm | |
| Negative | 495 [M-H]⁻ (100), | 583 [M-H]⁻ (25), 479 |
| FAB-MS | 479 (3) , 333 (4) , | [M-C₆H₅CO]⁻ (6) , 449 [M- |
| m/z (%) | 137 [HOC₆H₄COO]⁻ | C₆H₅COOCH₂]⁻ (4), 121 |
| | (36) . | [C₆H₅COO]^{- (100),} |
| | | 77[C₆H₅]⁻ (8) |

TABLE 5
¹³C and ¹H NMR spectrum data of paeoniflorin

Paeoniflorin

| | δ_{C} | δ_{H} |
|---|---|---|
| 1 | 89.04(s) | |
| 2 | 86.15(s) | |
| 3 | 44.88(t) | 2.48(d, J = 12.3 Hz) |
| | | 2.29(d, J = 12.3 Hz) |
| 4 | 106.09(s) | |
| 5 | 44.88(d) | 3.07(d, J = 6.6 Hz) |
| 6 | 71.84(s) | |
| 7 | 23.61(t) | 2.89(dd, J = 10.9, 6.6 Hz) |
| | | 2.31(d, J = 10.9 Hz) |
| 8 | 61.61(t) | 5.11(d, J = 12.1 Hz) |
| | | 5.22(d, J = 12.1 Hz) |
| 9 | 101.80(d) | 5.92(s) |
| 10 | 19.94(q) | 1.65(s) |
| 1′ | 100.59(d) | 5.15(d, J = 8.1 Hz) |
| 2′ | 75.09(d) | 7.02(t, J = 8.1 Hz) |
| 3′ | 78.51(d) | 3.18(t, J = 8.1 Hz) |
| 4′ | 71.76(d) | 4.16(t, J = 8.1 Hz) |
| 5′ | 78.61(d) | 3.92(DD, J = 8.1, 3.4 Hz) |
| 6′ | 62.94(t) | 4.54(dd, J = 11.7, 2.2 Hz) |
| | | 4.32(dd, J = 11.7, 5.7 Hz) |
| 1″ | 130.69(s) | |
| 2″, 6″ | 130.00 Cd) | 8.11(d, J = 7.8 Hz) |
| 3″, 5″ | 128.88(d) | 7.29(t, J = 7.8 Hz) |
| 4″ | 133.40 Cd) | 7.45(t, J = 7.8 Hz) |
| 7″ | 166.72(s) | |
| 1′″ | | |
| 3′″, 5 ′″ | | |
| 4′″ | | |
| 7′″ | | |

TABLE 6
¹³C and ¹H NMR spectrum data of oxypaeoniflorin

Oxypaeoniflorin

| | δ_{C} | δ_{H} |
|---|---|---|
| 1 | 89.00(s) | |
| 2 | 86.08(s) | |
| 3 | 44.89(t) | 2.47(d, J = 12.3 Hz) |
| | | 2.28(d, J = 12.3 Hz) |
| 4 | 106.05(s) | |
| 5 | 43.99(d) | 3.08(d, J = 6.3 Hz) |
| 6 | 71.84(s) | |
| 7 | 23.62(t) | 2.89(dd, J = 10.8, 6.3 Hz) |
| | | 2.31(d, J = 10.8 Hz) |
| 8 | 61.01(t) | 5.11(d, J = 12.1 Hz) |
| | | 5.22(d, J = 12.1 Hz) |
| 9 | 101.87(d) | 5.94(s) |
| 10 | 19.94(g) | 1.65(s) |
| 1′ | 100.62(d) | 5.16(d, J = 8.1 Hz) |
| 2′ | 75.12(d) | 4.04(t, J = 8.1 Hz) |
| 3′ | 78.48(d) | 4.23(t, J = 8.1 Hz) |
| 4′ | 71.84(d) | 4.19(t, J = 8.1 Hz) |
| 5′ | 78.62(d) | 3.93(dd, J = 8.1, 3.4 Hz) |
| 6′ | 62.94(t) | 4.54(dd, J = 11.7, 2.3 Hz) |
| | | 4.33 (dd, J = 11.7, 5.6 Hz) |
| 1″ | 130.69(s) | |
| 2″, 6″ | 130.00(d) | 8.14(d, J = 8.6 Hz) |
| 3″, 5″ | 128.88(d) | 7.06(d, J = 8.6 Hz) |
| 4″ | 133.40(d) | |
| 7″ | 166.72(s) | |

TABLE 7
¹³C and ¹H NMR spectrum data of benzoylpaeoniflorin

Benzoylpaeoniflorin

| | δ_{C} | δ_{H} |
|---|---|---|
| 1 | 89.01(s) | |
| 2 | 86.06(s) | |
| 3 | 44.77(t) | 2.39(d, J = 12.3 Hz) |
| | | 2.27(d, J = 12.3 Hz) |
| 4 | 106.00(s) | |
| 5 | 43.84(d) | 3.03(d, J = 6.5 Hz) |
| 6 | 71.43(s) | |
| 7 | 22.92(t) | 2.83(dd, J = 10.7, 7.0 Hz) |
| | | 2.17(d, J = 10.7 Hz) |
| 8 | 61.44(t) | 5.01(d, J = 12.1 Hz) |
| | | 5.15(d, J = 12.1 Hz) |
| 9 | 101.70(d) | 5.90(s) |
| 10 | 19.85(g) | 1.65(s) |
| 1′ | 100.36(d) | 5.11(d, J = 7.8 Hz) |
| 2′ | 74.91(d) | 4.02(t, J = 7.8 Hz) |
| 3′ | 78.27(d) | 4.19(t, J = 7.8 Hz) |
| 4′ | 71.78(d) | 4.06(t, J = 7.8 Hz) |
| 5′ | 75.14(d) | 4.09(dd, J = 7.8,4.5 Hz) |
| 6′ | 65.19(t) | 4.95(dd, |
| | | J = 11.5, 6.2 Hz) |
| | | 5.17(d, J = 11.5 Hz) |
| 1″ | 130.69(s) | |
| 2″, 6″ | 130.00(d) | 8.08(d, J = 8.0 Hz) |
| 3″, 5″ | 128.88(d) | 7.27(t, J = 8.0 Hz) |
| 4″ | 133.40(d) | 7.42(t, J =0 8.0 Hz) |
| 7″ | 166.72(s) | |
| 1′″ | 130.91(s) | |
| 2′″, 6′″ | 130.07(d) | 8.22(d, J = 7.8Hz) |
| 3′″, 5′″ | 128.93(d) | 7.38(t, J = 7.8 Hz) |
| 4′″ | 133.46(d) | 7.47(t, J =0 7.8 Hz) |
| 7′″ | 166.51(s) | |

Requirements for Materials and Reagents Used in Production

1) White peony root (Radix Paeoniae Alba): the crude drug for TGP product is from Bozhou, China Release criteria: Chinese Pharmacopoeia Volume 1 (1990 Edition) P 85

2) Chemical reagents for the product are industrial pure in purity grade:

95% Alcohol

Ethyl acetate

Sodium hydrogen carbonate

3) Water used in production is purified unpolluted living water

New Compound

Samples

TGP sample I was provided by the inventor.

TGP sample II was prepared in another laboratory with the above-described method.

Both samples appeared as a yellowish-brown powder, soluble in methanol to form a reddish brown solution.

Method:

Use reverse SiO2 Column, Lobar Column, HPLC, and Duplicated Crystallization to isolate a group of chemical compounds. FIGS. 34, 35 and 36 show the flow charts for analyzing both Sample I and Sample II, while Sample II was prepared from the white paeony root.

The chemical structures of chemical compounds I-XV isolated from both samples are shown in FIG. 38 and the below table.

Results:

A group of chemical compounds (chemicals I˜XV, some of them share similar structures) were isolated from the samples.

Chemical Structures of chemical compounds isolated from TGP (see FIG. 38 for chemical structures)

| | | ‘-R’ in | | |
|---|---|---|---|---|
| Chemical | Chemical | chemical | Sample | Sample |
| compounds | Structures | structure | I | II |
| I | | CH₃ | + | + |
| II | FIG. 38(1) | CH₂CH₃ | + | + |
| III | | CH₂CH₂CH₃ | + | + |
| IV | | | + | + |
| V | FIG. 38(2) | Phenyl | − | + |
| VI | FIG. 38(3) | | + | + |
| VII | FIG. 38(2) | H | + + | + + |
| VIII | FIG. 38(4) | H | + | + |
| IX | FIG. 38(4) | 3,4,5-trihydro- | + | + |
| | | phenyl | | |
| X | FIG. 38(2) | 3,4,5-trihydro- | + | + |
| | | phenyl | | |
| XI | FIG. 38(4) | p-hydro-phenyl | + | + |
| XII | FIG. 38(2) | p-hydro-phenyl | + | + |
| XV | FIG. 38(2) | Ac | + | − |

Some of the single ingredients obtained from TGP sample I and II in analysis procedures:

| No. | Name of Chemicals | Amount | Method |
|---|---|---|---|
| 1 | Paeoniflorin | 200 mg | HPLC |
| 2 | Albiflorin | 20 mg | HPLC |
| 3 | Ethane Paeoniflorin | 10 mg | HPLC ODS |
| | | | Column |
| 4 | Paeoniflorin R1 | 10 mg | HPLC |
| 5 | Benzoylpaeoniflorin | 40 mg | HPLC |

New Chemical Compound

A new chemical compound—PL 280 (Paeoniflorin R1) was discovered. Paeoniflorin R1 is a side product obtained from during the preparation of Total Glycosides of Paeony (TGP) and is one of the components of TGP. The given chemical name is: 2-hydroxy-3 Methyl-Glucoside-4-Oxo-7-Carbonyl-Tricyclo [3,2,2,0^{1,2}]-Nonanyl-Benzoicmethoxycarbonyl. The structure of PL 280 is shown in FIG. 37. The method or procedure to obtain paeoniflorin R1 from white paeony root is similar to that for TGP and is shown in FIG. 39.

Properties of PL 280

PL 280 (Paeoniflorin R1) is a white powder, positive in glasses reaction. It turns red-violet in sulfuric acid reaction, and its UV (λₘₐₓ^{MeOH}): 228.6 nm, IR (cm⁻¹): 3536 (OH), 2931 (CH₂), 1747, 1731, 1712 (C═O), 1624, 1450 (phenyl), 1383 (CH₃), 1275 (O—C—O), 1116 (C—O), 717

Determination of the Structure

In ¹HNMR spectrum (300 MHz, CD₃OD), groups of signals were detected:

δ 7.97 (2H, d, J=7.5 Hz), 7.63 (1H, t, J=7.5 Hz), 7.49 (2H, t, J=7.5 Hz);(protons in single phenyl replacement)

δ4.54 (1H, d, J=8.0 Hz, glc-1′H), 3.26˜3.95 (6H, m, glc-H); (protons in the glucose)

δ1.55 (3H, s); (protons in methyl)

δ4.93 (1H, br.s), 4.78 (2H, s); (proton on carbons bond to oxygen)

δ2.69 (2H, br.s), 2.80 (1H, dd, J=8.1, 4.8 Hz), 2.60 (1H, t, J=8.1 Hz), 2.25 (1H, dd, J=8.1, 4.8 Hz). (alkyl protons)

In ¹³CNHR spectrum (75.4 MHz, CD₃OD), groups of signals were detected:

21 carbon signals, include the glucose carbons at: δ695.00, 74.33, 72.97, 70.09, 77.87, 61.09;

δ166.17 (C═O), 133.35 (4″C), 129.23 (2″, 6″C), 128.40 (1″C), 128.51 (3″,5″C); (protons on benzene meth-alkene)

δ216.47; (carbonyl carbon)

δ102.76, 85.17, 80.89, 62.84; (carbons bond to oxygen)

δ55.18, 47.35, 37.33, 30.44, 15.63. (alkyl carbons)

By analyzing the above signals with the satellite peaks in HMBC spectrum, the following clues were found:

δ4.78 proton is on δ62.84 carbon;

δ1.55 (3H, s) is a methyl proton on 615.63 carbon;

δ2.80 proton is on δ37.33 carbon;

δ2.25 and δ2.60 protons are both on δ30.44 carbon;

δ2.69 (2H) proton is on δ47.75 carbon;

δ4.93 (1H, br.s) proton is on δ80.89 carbon.

In ¹H-¹H COSY spectrum:

δ2.25, 2.60 correlate with δ2.80;

δ2.69 (2H) and δ4.93 (1H, s) have remote correlation and are protons in the glucose.

In HMBC spectrum:

δ4.78 (2H, br.s, CH₂O) remotely correlate with δ166.17 carbon and δ55.18, 80.89, 85.17, 37.33; 61.55 (3H, s) methyl proton remotely correlates with δ102.76 and 85.17, carbons bond to oxygen. Presumably, the section is connected with δ85.17 carbon, hence, section [A] exists (FIG. 42, A).

δ2.80 remotely correlates with carbons δ216.69, as well as δ47.75, 30.44, 85.17; protons δ2.25 and 2.60 both remotely correlate with δ216.69, 37.33, 85.17; (in addition, δ2.69 remotely correlates with δ37.33), it is deducted that section [B] exists (FIG. 42, B).

δ2.80, 2.25 in section [B] remotely correlate with δ55.18 carbon in section [A]; δ 2.69 in section [B] remotely correlates with δ80.89 in section [A]. It is presumed that section [A] and [B] are connected in the way shown in FIG. 41.

δ3.30, the proton at the 2^{nd} site of the glucose remotely correlates with δ102.76, indicating that the site is connected to a structure with a ketone bond;

The signal of carbon at the 2^{nd} site of the glucose moves toward a lower electromagnetic field to δ74.33, while carbon at 1^{st} and 3^{rd} site towards a higher electromagnetic field to δ95.00 and δ72.97. It is presumed that certain sites of the glucose are replaced, the glasses reaction is positive (reductive), all these indicate the free end of the glucose.

The compound's MS spectrum has a false peak of molecular ion 481 [M+H]⁺, which supports the established structure. (FIG. 40).

In NOESY spectrum:

δ1.55 (3H, s) methyl proton signal correlates with δ2.25 proton in NOE; and there is NOE relation between the glucose-end proton and δ4.78 (2H), br, s, CH₂OBz). It is presumable that the methyl structure is located at the inner side of the “basket structure.” With many other support NOE signals, the structure of PL 280 is established as shown in 43.

Pharmacodynamics of the New Chemical Compound

The new chemical compound, Paeoniflorin R1, was also demonstrated to be effective in treating inflammatory conditions and immune system disorders.

Animal: Wistar rats, 2-3 months, 180±30 g.

Chemical agents: Paeoniflorin R1, Lobenzarit disodium (CCA), Indomethacin (IM), and BCG vaccine

Model of Arthritis: inject 0.1 ml of 10 mg BCG vaccine to the right hind foot, bottom side, intracutaneously.

Observations

Primary inflammation: Degree of swelling of injected feet (primary inflammation): oral treatment was given 30 minutes before the inflammatory injection (BCG vaccine). The degree of swelling was measured 1, 3, 5, and 7 days after the injection.

Groups of animals for secondary inflammation: 4 groups, each contains 10 rats, groups are: 1) Normal control, receive inflammatory injection and no treatment; 2) Positive control, receive inflammatory injection and IM or CCA treatment; 3) TGP treatment group: receive inflammatory injection and TGP 25 mg/Kg/d. Oral administration of Paeoniflorin R1 30˜60 minutes before inflammatory injection.

TGP's influence on degree of swelling of injected feet (primary inflammation)

| | Dosage | Number | | | | |
|---|---|---|---|---|---|---|
| | (mg/K/g/ | of | Degree of Swelling | | | |
| Groups | d) | Animals | Day 1 | Day 3 | Day 5 | Day 7 |
| Control | | 10 | 0.62 ± | 1.18 ± | 0.86 ± | 0.54 ± |
| | | | 0.18 | 0.11 | 0.22 | 0.08 |
| Paeoniflorin | 25 | 10 | 0.59 ± | 0.81 ± | 0.50 ± | 0.40 ± |
| R1 | | | 0.20 | 0.18** | 0.14** | 0.16** |
| IM | 2 | 10 | 0.52 ± | 0.57 ± | 0.34 ± | 0.30 ± |
| | | | 0.21 | 0.12** | 0.08** | 0.09** |
| CCA | 50 | 10 | 0.65 ± | 0.96 ± | 0.68 ± | 0.65 ± |
| | | | 0.23 | 0.19 | 0.18 | 0.18 |

*P < 0.05
**P < 0.01

Secondary inflammation: Swelling degree of the opposite non-injected feet (secondary inflammation): oral treatment was given 12-28 days after the inflammatory injection. The degree of swelling was measured 12, 16, 20, 24 and 28 days after the injection.

Groups of animals for secondary inflammation: 6 groups, each contains 6 rats, groups are: 1) Normal control, receive no inflammatory injection and any pre treatment; 2) Negative control, receive inflammatory injection and no treatment; 3) Positive control, receive inflammatory injection and CCA treatment; 4) Paeoniflorin R1 groups (5, 25, 50 mg/kg/d) oral administration of Paeoniflorin R1 30˜60 minutes before inflammatory injection.

TGP's influence on degree of swelling of opposite, non-injected feet (secondary inflammation)

| | | | | | | | | W. of |
|---|---|---|---|---|---|---|---|---|
| | | | | | | | | Thymus |
| | | | | | | | | Glands |
| | Dosage | No. | Degree of Swelling | | | | | (mg/kg |
| | (mg/k | of | Day | Day | Day | Day | Day | body |
| Groups | g/d) | Animals | 12 | 16 | 20 | 24 | 28 | W.) |
| Normal | | 6 | | | | | | |
| Control | | | | | | | | |
| N.Control | | 6 | | 0.41 ± | 0.57± | 0.76 ± | 0.60 ± | 9.7 ± 2.2 |
| | | | | 0.10 | 0.16 | 0.38 | 0.30 | |
| Paeoniflorin | 5 | 6 | 0.35 ± | 0.42 ± | 0.33 ± | 0.25 ± | 0.26 ± | 16.3 ± |
| | | | 0.08 | 0.13 | 0.14** | 0.15** | 0.16** | 3.3*1* |
| R1 | 25 | 6 | 0.41± | 0.39 ± | 0.20 ± | 0.15 ± | 0.23 ± | 19.4 ± |
| | | | 0.09 | 0.13 | 0.07** | 0.08** | 0.10** | 6.0** |
| | 50 | 6 | 0.40 ± | 0.39 ± | 0.30 ± | 0.27 ± | 0.24 ± | 13.9 ± |
| | | | 0.12 | 0.16 | 0.08** | 0.09** | 0.14** | 2.4** |
| CCA | 50 | 6 | 0.40 ± | 0.55 ± | 0.42 ± | 0.14 ± | 0.05 ± | 14.4 ± |
| | | | 0.08 | 0.24 | 0.23* | 0.14** | 0.02** | 3.8** |

*P < 0.05
**P < 0.01

Amount of H2O2, IL-1, and PGE2 produced by abdominal cavity macrophage: Use 5 rats in each group, paeoniflorin R1 (25 mg/kg) treatment starts 7 days after inflammatory injection, animal sacrificed on day 17. The result is shown in FIG. 44: the drug can decrease the above reaction.

Sub-Group T Cell in Peripheral Circulation

Paeoniflorin R1 treatment (25 mg/kg) started at 18 days after inflammatory injection, animal sacrificed on day 26. The result showed the drug can improve Th/Ts (P<0.01) (FIG. 45).

Kinemics in Abdominal Cavity Macrophage Release PGE2

Paeoniflorin R1 treatment started the 1st day after inflammatory injection, animal sacrificed on day 7, 14, 21, and 28. FIG. 46 shows that Paeoniflorin R1 and IM are effective in PGE2 release.

IL-1, TNF, and PGE2 Produced by Synovial Cells

Paeoniflorin R1 treatment (25 mg/kg) started at 12 days after inflammatory injection, animal sacrificed on day 22. The result showed the drug can improve Th/Ts (P<0.01)

Laboratory study has proven Paeoniflorin R1 is a biologically active agent in inhibiting inflammation and moderate immune system activities.

Quality Control for Intermediate

(1) Standard and Reference: Chinese Pharmacopoeia, Edition 1995; Standard of Health Ministry of China (trying out.) WS-044 (X-034)₉₅

(2) Appearance: powder TGP is a light yellowish-brown powder; tastes slightly bitter, sour and puckery. With hygroscopicity, it is soluble in water, ethanol and ethyl acetate, slight soluble in diethyl ether and chloroform.

(3) Method of Identification: Dissolve 1˜2 mg sample in 1 ml acetic anhydride and add 4-5 drops of sulfuric acid into the solution. It turns red or red violet.

Weigh 2 mg of sample accurately and dissolve it evenly in 1 ml of methanol, prepare reference solution of Paeoniflorin to make it 1 mg/ml. Add 5 ul of both solutions onto a silica gel plate in a fluid phase of chloroform-methanol (chloroform:methanol=5:1; refer to Appendix of Chinese Pharmacopoeia 1995 Edition for method and procedure of TLC). Use 10% sulfuric acid as color developer. A spot in the same color and at about the same height with those of the reference can be observed.

(4) Acidity: Dissolve 0.1 g of sample into 50 ml of water. The PH value of such solution should be in a range from 5.0 to 6.0 (refer to Appendix of Chinese Pharmacopoeia 1995 Edition for method of PH value test)

(5) Contaminations test: Add 10 mg of sample into 1 ml of methanol to make the sample solution. Prepare the reference solution and develop both in the same way with the above item (3). The spot of impurity in sample should not be deeper than that of the reference.

(6) Weight loss in drying: bake the drug in 105° C. until it comes to a constant weight. No less than 4.5 percent of weight loss permitted.

(7) Heavy metals: prepare a 25 ml solution from 1.0 g of sample. No more than 10 ppm of heavy metals is allowed (refer to Appendix of Chinese Pharmacopoeia 1995 Edition for method of heavy metals test)

(8) Ignition residue: No more than 1.0% of ignition residue is allowed (refer to Appendix of Chinese Pharmacopoeia 1995 Edition for test method)

(9) Microorganism limit examination

Total bacterium less than 500 per gram

Total fungus less than 500 per gram

No found of Bacillus coli

(10) Content of paeoniflorin

System Applicability of HPLC:

Packed material: Octadecyl silane linkage silica

Flow phase: methanol-water (24:76)

Flow speed: 1.5 ml/min

Detective wave-length: 230 nm

Sensitivity: 0.16 A.U.F.S.

Number of column plates: no less than 800

Injection: 4 μl of both solutions

Method of calculation: external standard method.

Reference solution: accurately weigh 10 mg of paeoniflorin in a measuring flask. Add 50% methanol solution to the scale and shake to make it thoroughly mixed. The solution is 1 mg/ml.

Sample solution: prepare the sample solution with 20 mg accurately weighted sample and 50% methanol in the same way with the reference.

To control batch variation, paeoniflorin is chosen for an important index of TGP since it is relatively the most stable and permanent content in comparison with the other glycosidess. The peaks of HPLC reflect the overall content of TGP. The total glycosides extracted from peony (TGP), should contain no less than 40 percent of paeoniflorin in the dry material.

Quality Control for Final Product

Appearance of capsule: hard capsule filled with powder TGP or recipient (used for the control group in clinical trials). The capsule of TGP looks like a normal capsule filled with brown powder.

Weight of content: the amount of powder TGP in each capsule should be 90.09 to 110.0% of labeled amount (300 mg).

Dissolution of Capsule:

Immerse the sample in solvent water in a dissolution machine that is on the rotation of 100 per minute. Sample 5 ml of solutions at 10^{th} and 40^{th} minute after the machine is started and put them into 100 ml measure flasks. Add ethanol to dilute the sample solutions to the scale and shake. Examine their absorptions at 220 nm in UV spectrum to calculate the rate of dissolution. The dissolution ratio should be 80.0% or more. (Refer to Appendix of Chinese Pharmacopoeia, 1990 Edition for dissolution test). Dissolution Ratio can be calculated as:

adsorption of sample in 10^{th} minutes/adsorption of sample in 40^{th} minute X 100%

Identity of Capsules

Weigh 20 whole capsules, and their shells respectively to calculate the weight of content in each capsule and the mean weight. No more than two capsules may be out of the limit of content weight (90%˜110%). No capsule can be found beyond two times of the limit. If the mean weight is below 0.3 g, the limit of difference is 10%; if it is more than 0.3 g, the limit should be 7.5%.

HPLC Fingerprint of Total Glycosides of Paeony (TGP)

Analysis title: Chromatographic fingerprint of Total Glycosides of Paeony by High Performance liquid chromatography.

Scope: The fingerprint established can be used to evaluate the quality and consistency of the total glycosides of Paeony from batch to batch.

Principle: Samples are dissolved in methanol as sample solutions. The sample solutions are analyzed with High Performance liquid Chromatography (HPLC) using Lichrospher 100RP-18 column and detected by Diod Array Detector at 230 nm.

A 3D chromatogram is also carried out. By comparing the samples with a common pattern of the fingerprint, the batch-to-batch consistency of the samples can be judged.

Apparatus: Calibrated analytical balance accurate to 0.1 mg Lichrospher 100 RP-18, 5 μm, 4×125 mm column

High performance liquid chromatographic system, Agilent Model 1100 equipped with an auto sampler, Diod Array Detector and works on Chemo station platform

Reagents: Water, liquid chromatographic grade for mobile phase; water, deionized for sample solution preparation; Methanol, liquid chromatographic grade; phosphoric acid, concentrated, analytical grade; and acetonitrile, liquid chromatographic grade.

Samples: 10 batches of Total glycosides of Paeony (TGP) Batch numbers: 010814, 010828, 010911, 010928, 011019, 011109, 011122, 011204, 011221, 020118.

Reference substances: Paeoniflorin, Albiflorin, and Benzoxylpaeoniflorin

Referene solution preparation: Weigh the reference substances individually and dissolved into 0.5% methanol to make the volume to 1 mg per ml. for paeoniflorin, 0.5 mg per ml. for Albiflorin and 0.2 mg per ml. for Benzoylpaeoniflorin respectively. Store the reference solution in a tightly sealed glass vial at lower than 4° C. in refrigerator. It can be used within 1 month. The HPLC of the reference substances is shown in FIG. 47.

Sample solution preparation: Weigh accurately 20 mg of the dry extract sample, dissolve it in 10 ml. of 0.5% methanol, filter it through a 0.45 μm filter membrane. The filtrates are sample solutions. The HPLC of all samples is shown in FIGS. 52 and 53.

Chromatographic Condition:

Column: Lichrospher 100 RP-18, 4×125 mm

Column temperature: 20° C.

Gradient of mobile phase:

| | percent 0.1% | percent |
|---|---|---|
| Time (min) | phosphoric acid | Acetonitrile |
| 0 | 90% | 10% |
| 15 | 60% | 40% |

Flow rate (mL/min): 1.0

Injection volume: 5 μL

Detection wavwlenghth: 230 nm

Run time: 16 minutes

Relative Retention Time of Reference Substances:

| | |
|---|---|
| Albiflorin | 0.9 |
| Paeoniflorin | 1.0 |
| Benzoylpaeoniflorin | 2.2 |

Procedures: Prepare reference solution and sample solutions as aforementioned described.

Prepare a blank solvent of 0.5% methanol.

Make a single injection of the blank.

Make a single injection of the reference solutions.

Make a single injection of mixed reference solution (Mix Paeoniflorin, Albiflorin and Benzoylpaeoniflorin Reference solutions, 1 mL for each, shake thoroughly in a tightly sealed vial).

Make a single injection of the sample solutions with different batch numbers.

Make a single injection of mixed sample solution of 10 batch numbers sample solution (Mix the same volume of the 10 batch numbers of sample solutions and shake thoroughly).

Record the chromatograms through WorkStation respectively.

Take the chromatogram of the mixed sample solution as a common pattern of the fingerprint of TGP, i.e., Reference Fingerprint of TGP.

Observe and compare of the chromatograms of the sample solutions respectively against the Reference fingerprint of TGP.

Using the above circumstances, the HPLC fingerprint of TGP is established. It may be used for the quality control of TGP product. The fingerprint of TGP is as shown in FIG. 48, and its 3D chart is in FIG. 49. The table below shows the variation of retention times for the 8 characteristic peaks between 10 batches.

| BATCH | PEAK NUMBER | | | | | | | |
|---|---|---|---|---|---|---|---|---|
| # | 1 | 2 | 3 | 4 | 5 | 6 | 7 | 8 |
| 010928 | 0.73 | 0.91 | 1 | 1.12 | 1.29 | 1.37 | 1.54 | 2.16 |
| 011204 | 0.73 | 0.91 | 1 | 1.12 | 1.29 | 1.37 | 1.54 | 2.16 |
| 010814 | 0.73 | 0.91 | 1 | 1.12 | 1.29 | 1.37 | 1.54 | 2.16 |
| 011109 | 0.73 | 0.91 | 1 | 1.12 | 1.29 | 1.37 | 1.54 | 2.16 |
| 011221 | 0.73 | 0.91 | 1 | 1.12 | 1.29 | 1.37 | 1.54 | 2.16 |
| 011019 | 0.73 | 0.90 | 1 | 1.12 | 1.29 | 1.37 | 1.54 | 2.16 |
| 010828 | 0.73 | 0.91 | 1 | 1.12 | 1.29 | 1.37 | 1.54 | 2.16 |
| 020118 | 0.73 | 0.91 | 1 | 1.12 | 1.29 | 1.37 | 1.54 | 2.16 |
| 010911 | 0.73 | 0.91 | 1 | 1.12 | 1.29 | 1.37 | 1.54 | 2.16 |
| 011122 | 0.73 | 0.91 | 1 | 1.12 | 1.29 | 1.37 | 1.54 | 2.16 |

Note: The relative retention time of Paeoniflorin (peak 3) is considered as “1”, which is the basis of other data.

There are approximately 8 peaks in the HPLC fingerprint of TGP. The characteristic of HPLC fingerprint of TGP is that Paeoniflorin (peak 3) dominates the total chromatogram. Peak 2 is albiflorin and peak 8 is benzoylpaeoniflorin, the others are unknown glycosides. Assume peak 3 (paeoniflorin) is 1, the relative ratio of all of the peaks height are peak 1: peak 2 (albiflorin):peak 3 (paeoniflorin): peak 4: peak 5: peak 6: peak 7: peak 8 (benzoylpaeoniflorin)=0.05: 28: 1.0: 0.03: 0.03: 0.15: 0.03: 0.04 (FIG. 50). The main characteristic peaks in the fingerprint are peaks 3, 2, 6, 1 and 8.

The data of peak area and height are shown in the two below tables. The relative charts for the peak areas and heights among the 10 batches are shown in FIG. 51. A±10% limitation of all of the integration data (peak areas and peak heights) is allowed among the different batches of TGP samples.

Peak areas of the 8 peaks in HPLC fingerprint for 10 batches

| Batch | Peak | Peak | Peak | Peak | Peak | Peak | Peak | Peak |
|---|---|---|---|---|---|---|---|---|
| No. | 1 | 2 | 3 | 4 | 5 | 6 | 7 | 8 |
| 010928 | 429 | 1712 | 5693 | 177 | 224 | 1010 | 218 | 250 |
| 011204 | 418 | 1609 | 5329 | 176 | 210 | 1065 | 220 | 226 |
| 010814 | 402 | 1652 | 5491 | 174 | 211 | 1041 | 238 | 235 |
| 011109 | 368 | 1638 | 5491 | 172 | 212 | 961 | 232 | 235 |
| 011221 | 361 | 1644 | 5518 | 173 | 213 | 973 | 248 | 236 |
| 011019 | 410 | 1774 | 5947 | 191 | 282 | 1012 | 184 | 260 |
| 010828 | 352 | 1655 | 5594 | 175 | 216 | 973 | 249 | 241 |
| 020118 | 315 | 1559 | 5275 | 161 | 204 | 852 | 224 | 226 |
| 010911 | 312 | 1520 | 5153 | 160 | 197 | 849 | 223 | 223 |
| 011122 | 290 | 1544 | 5293 | 157 | 205 | 772 | 200 | 229 |

Peak heights of the 8 peaks in HPLC fingerprint for 10 batches

| Batch | Peak | Peak | Peak | Peak | Peak | Peak | Peak | Peak |
|---|---|---|---|---|---|---|---|---|
| No. | 1 | 2 | 3 | 4 | 5 | 6 | 7 | 8 |
| 010928 | 54 | 224 | 762 | 24 | 25 | 137 | 27 | 30 |
| 011204 | 51 | 209 | 715 | 24 | 23 | 142 | 26 | 28 |
| 010814 | 50 | 219 | 748 | 23 | 23 | 142 | 29 | 30 |
| 011109 | 46 | 216 | 745 | 23 | 24 | 132 | 28 | 30 |
| 011221 | 46 | 213 | 737 | 23 | 23 | 130 | 30 | 29 |
| 011019 | 53 | 232 | 804 | 25 | 27 | 133 | 24 | 33 |
| 010828 | 45 | 214 | 745 | 24 | 24 | 130 | 30 | 30 |
| 020118 | 41 | 202 | 707 | 22 | 22 | 115 | 27 | 28 |
| 010911 | 40 | 200 | 701 | 22 | 21 | 114 | 27 | 28 |
| 011122 | 38 | 201 | 709 | 21 | 22 | 104 | 24 | 29 |

Relative Retention Time: Assume peak 3 (paeoniflorin) is 1 for the retention time, the values of the relative retention time peak 1 through 8 are:

| Peaks | Relative Retention Time |
|---|---|
| 1 | 0.73 |
| 2 | 0.91 |
| 3 | 1 |
| 4 | 1.12 |
| 5 | 1.29 |
| 6 | 1.37 |
| 7 | 1.54 |
| 8 | 2.16 |

The 10 batches of samples' chromatograms are closely compared with the TGP fingerprint (FIG. 48).

The HPLC of the 10 batches of TGP samples are very similar to the TGP fingerprint. Among the samples analyzed, they have very high similarity (FIGS. 51, 52, and 53). This demonstrates that the quality of the 10 batches of samples analyzed is consistent.

Stability Tests for Bulk Drug TGP

Six batches of TGP bulk drug (batch number: 903014, 903015, 903016, 921019, 921020, and 921021) were tested for the stability in different conditions (36 months, sealed, room temperature; 12 months, not sealed, room temperature; 10 days in temperature of 40° C., 60° C. and 80° C.; and 10 days in forceful light radiation of 2000 Lx and 4000 Lx)

Sampling and Storing Conditions:

Stability assay for drug stored at room temperature: weighed 50 grams of bulk TGP drug from each of the three batches: 903014, 903015 and 903016. Put them into 10 sealed glass bottles; Weighed 30 grams of bulk TGP drug from the same batches of drug and put them into another 10 containers (culture plates) which were not sealed. Sampling and testing them periodically in 0, 1^{st}, 2^{nd}, 3^{rd}, 6^{th}, 12^{th}, 18^{th}, 24^{th}, and the 36^{th} month for sealed samples or 1^{st}, 2^{nd}, 3^{rd}, 6^{th}, 12^{th} month for unsealed samples. The results are listed in the tables below.

Stability assay for drugs stored in higher temperatures (accelerated test): Weighed 15 grams of bulk TGP drug from each of the three batches: 921019, 921020, and 921021. Spread them out on a clean glass plate to form a layer of about 5 mm thick. Put the plates in a constant temperature heater for ten days, the temperature was set to 40° C., 60° C. and 80° C. Samples are taken and tested in 0, 1^{st}, 3^{rd}, 5^{th}, and the 10^{th} days

Stability assay for drug under forceful light radiation (accelerated test): Weighed 15 grams of bulk TGP drug from each of the three batches: 921019, 921020, and 921021. Spread them on a plain glass plate to form a layer of about 5 mm thick. Put the plates under a forceful light of 2000 Lx or 4000 Lx in the clean work-bench which was covered around with black curtains. Samples were taken periodically and tested in 0, 1^{st}, 3^{rd}, 5^{th}, and the 10^{th} days

Test Items and Methods:

1) Organoleptic analysis: Appearance inspection

2) Identification of Paeoniflorin in TGP samples: Follow step 1 and step 2 in section 1.4.4. (quality control system for manufacture/release criteria and tests) the result of this test item can only be positive or negative.

3) Measuring the amount of Paeoniflorin: Follow instructions in section 1.4.4 for system standard of HPLC, preparation of reference and sample solutions for injection, and the principle for calculation. Result is presented in percentages.

4) Weight lost after drying in the thermostat: Weigh precisely 1 gram of TGP. Heat it in a thermostat of 105° C., until it comes to a constant weight. Calculate the weight lost. 5) Hygienical inspection: sterility examination

TABLE 8
Stability test at room temperature (sealed)

| | | | | Amount | Weight | |
|---|---|---|---|---|---|---|
| | | | | of | Lost | |
| Batch | | | Identi- | Paeon- | after | Hygienical |
| No | Month | Appearance | fication | iflorin | drying | inspection |
| 903014 | 0 | Light | + | 44.3 | 3.6 | Pass |
| | | yellowish | | | | |
| | | powder | | | | |
| | 1 | Same | + | 44.3 | 3.6 | Pass |
| | 2 | Same | + | 44.2 | 4.3 | Pass |
| | 3 | Same | + | 44.2 | 4.6 | Pass |
| | 6 | Same | + | 44.1 | 5.4 | Pass |
| | 12 | Same | + | 44.1 | 5.6 | Pass |
| | 18 | Same | + | 44.2 | 5.7 | Pass |
| | 24 | Same | + | 44.1 | 5.8 | Pass |
| | 36 | Same | + | 44.0 | 5.9 | Pass |
| 903015 | 0 | Light | + | 42.8 | 4.0 | Pass |
| | | yellowish | | | | |
| | | powder | | | | |
| | 1 | Same | + | 42.8 | 4.1 | Pass |
| | 2 | Same | + | 42.8 | 4.4 | Pass |
| | 3 | Same | + | 42.7 | 4.8 | Pass |
| | 6 | Same | + | 42.7 | 5.3 | Pass |
| | 12 | Same | + | 42.7 | 5.4 | Pass |
| | 18 | Same | + | 42.6 | 5.6 | Pass |
| | 24 | Same | + | 42.7 | 5.9 | Pass |
| | 36 | Same | + | 42.6 | 5.9 | Pass |
| 903016 | 0 | Light | + | 42.4 | 3.9 | Pass |
| | | yellowish | | | | |
| | | powder | | | | |
| | 1 | Same | + | 42.3 | 4.1 | Pass |
| | 2 | Same | + | 42.3 | 4.3 | Pass |
| | 3 | Same | + | 42.3 | 4.6 | Pass |
| | 6 | Same | + | 42.2 | 4.8 | Pass |
| | 12 | Same | + | 42.2 | 5.2 | Pass |
| | 18 | Same | + | 42.2 | 5.4 | Pass |
| | 24 | Same | + | 42.1 | 5.6 | Pass |
| | 36 | Same | + | 42.0 | 5.7 | Pass |

TABLE 9
Stability test in room temperature (not sealed)

| | | | | | Weight | |
|---|---|---|---|---|---|---|
| | | | | Amount | Lost | |
| Batch | | Ap- | Identifi- | of Paeon- | after | Hygienical |
| No. | Month | pearance | cation | iflorin | drying | inspection |
| 903014 | 0 | Light | + | 44.3 | 3.6 | Pass |
| | | yellowish | | | | |
| | | powder | | | | |
| | 1 | Same | + | 43.8 | 3.6 | Pass |
| | 2 | Caked | + | 43.1 | 4.3 | Pass |
| | | powder | | | | |
| | 3 | Caked | + | 42.0 | 4 6 | Pass |
| | | powder | | | | |
| | 6 | Shape | + | 41.6 | 5 4 | Pass |
| | | in starch | | | | |
| | | syrup | | | | |
| | 12 | Shape | + | 38.3 | 5.6 | Pass |
| | | in starch | | | | |
| | | syrup | | | | |
| 903015 | 0 | Light | + | 42.8 | 4.0 | Pass |
| | | yellowish | | | | |
| | | powder | | | | |
| | 1 | Same | + | 42.3 | 4.1 | Pass |
| | 2 | Caked | + | 41 8 | 4.4 | Pass |
| | | powder | | | | |
| | 3 | Caked | + | 40.9 | 4.8 | Pass |
| | | powder | | | | |
| | 6 | Shape in | + | 40.0 | 5.3 | Pass |
| | | starch | | | | |
| | | syrup | | | | |
| | 12 | Shape in | + | 36.9 | 5.4 | Pass |
| | | starch | | | | |
| | | syrup | | | | |
| 903016 | 0 | Light | + | 42.4 | 3.9 | Pass |
| | | yellowish | | | | |
| | | powder | | | | |
| | 1 | Same | + | 41.8 | 4.1 | Pass |
| | 2 | Caked | + | 41.2 | 4.3 | Pass |
| | | powder | | | | |
| | 3 | Caked | + | 40.1 | 4.6 | Pass |
| | | powder | | | | |
| | 6 | Shape in | + | 39.3 | 4.8 | Pass |
| | | starch | | | | |
| | | syrup | | | | |
| | 12 | Shape in | + | 36.5 | 5.2 | Pass |
| | | starch | | | | |
| | | syrup | | | | |

TABLE 10
Stability test in 80° C.

| | | | | | Weight | |
|---|---|---|---|---|---|---|
| | | | | | Lost | |
| Batch | | | Identifi- | Amount of | after | Hygienical |
| No. | Days | Apearance | cation | Paeoniflorin | drying | inspection |
| 921019 | 0 | Light | + | 44.2 | 3.9 | Pass |
| | | yellowish | | | | |
| | | powder | | | | |
| | 1 | Same | + | 44.3 | 3.9 | Pass |
| | 3 | Same | + | 44.2 | 3.8 | Pass |
| | 5 | Same | + | 44.1 | 3.6 | Pass |
| | 10 | Same | + | 44.0 | 3.0 | Pass |
| 921019 | 0 | Light | + | 43.8 | 3.8 | Pass |
| | | yellowish | | | | |
| | | powder | | | | |
| | 1 | Same | + | 43.8 | 3.8 | Pass |
| | 3 | Same | + | 43.6 | 3.7 | Pass |
| | 5 | Same | + | 43.7 | 3.4 | Pass |
| | 10 | Same | + | 43.6 | 3.2 | Pass |
| 921021 | 0 | Light | + | 44.8 | 3.9 | Pass |
| | | yellowish | | | | |
| | | powder | | | | |
| | 1 | Same | + | 44.8 | 3.9 | Pass |
| | 3 | Same | + | 44.7 | 3.6 | Pass |
| | 5 | Same | + | 44.6 | 3.4 | Pass |
| | 10 | Same | + | 44.7 | 3.0 | Pass |

TABLE 11
Stability test in 60° C.

| | | | | | Weight | |
|---|---|---|---|---|---|---|
| | | | | | Lost | |
| Batch | | Ap- | Identifi- | Amount of | after | Hygienical |
| No. | Days | pearance | cation | Paeoniflorin | drying | inspection |
| 921019 | 0 | Light | + | 44.6 | 3.9 | Pass |
| | | yellowish | | | | |
| | | powder | | | | |
| | 1 | Same | + | 44.6 | 3.8 | Pass |
| | 3 | Same | + | 44.7 | 3.8 | Pass |
| | 5 | Same | + | 44.6 | 3.7 | Pass |
| | 10 | Same | + | 44.4 | 3.6 | Pass |
| 921019 | 0 | Light | + | 44.4 | 3.8 | Pass |
| | | yellowish | | | | |
| | | powder | | | | |
| | 1 | Same | + | 44.2 | 3.7 | Pass |
| | 3 | Same | + | 44.3 | 3.7 | Pass |
| | 5 | Same | + | 44.5 | 3.6 | Pass |
| | 10 | Same | + | 44.2 | 3.5 | Pass |
| 921021 | 0 | Light | + | 43.8 | 4.0 | Pass |
| | | yellowish | | | | |
| | | powder | | | | |
| | 1 | Same | + | 44.7 | 4.0 | Pass |
| | 3 | Same | + | 43.6 | 3.9 | Pass |
| | 5 | Same | + | 43.2 | 3.8 | Pass |
| | 10 | Same | + | 43.2 | 3.7 | Pass |

TABLE 12
Stability test in 40° C.

| | | | | | Weight | |
|---|---|---|---|---|---|---|
| | | | | | Lost | |
| Batch | | Ap- | Identifi- | Amount of | after | Hygienical |
| No. | Days | pearance | cation | Paeoniflorin | drying | inspection |
| 921019 | 0 | Light | + | 44.2 | 4.1 | Pass |
| | | yellowish | | | | |
| | | powder | | | | |
| | 1 | Same | + | 44.2 | 4.0 | Pass |
| | 3 | Same | + | 44.1 | 4.0 | Pass |
| | 5 | Same | + | 44.2 | 4.1 | Pass |
| | 10 | Same | + | 44.3 | 4.0 | Pass |
| 921019 | 0 | Light | + | 44.2 | 3.8 | Pass |
| | | yellowish | | | | |
| | | powder | | | | |
| | 1 | Same | + | 44.1 | 3.8 | Pass |
| | 3 | Same | + | 44.0 | 3.8 | Pass |
| | 5 | Same | + | 44.2 | 3.9 | Pass |
| | 10 | Same | + | 44.1 | 3.9 | Pass |
| 921021 | 0 | Light | + | 44.2 | 4.2 | Pass |
| | | yellowish | | | | |
| | | powder | | | | |
| | 1 | Same | + | 44.0 | 4.2 | Pass |
| | 3 | Same | + | 44.1 | 4.3 | Pass |
| | 5 | Same | + | 44.0 | 4.3 | Pass |
| | 10 | Same | + | 44.0 | 4.3 | Pass |

TABLE 13
Stability test in forceful light radiation of 2000 Lx

| | | | | | Weight | |
|---|---|---|---|---|---|---|
| | | | | | Lost | |
| Batch | | Ap- | Identifi- | Amount of | after | Hygienical |
| No. | Days | pearance | cation | Paeoniflorin | drying | inspection |
| 921019 | 0 | Light | + | 44.3 | 3.4 | Pass |
| | | yellowish | | | | |
| | | powder | | | | |
| | 1 | Same | + | 44.3 | 3.4 | Pass |
| | 3 | Same | + | 44.2 | 3.3 | Pass |
| | 5 | Same | + | 44.1 | 3.2 | Pass |
| | 10 | Same | + | 44.1 | 3.2 | Pass |
| 921019 | 0 | Light | + | 42.8 | 3.6 | Pass |
| | | yellowish | | | | |
| | | powder | | | | |
| | 1 | Same | + | 42.8 | 3.5 | Pass |
| | 3 | Same | + | 42.7 | 3.5 | Pass |
| | 5 | Same | + | 42.6 | 3.4 | Pass |
| | 10 | Same | + | 42.6 | 3.4 | Pass |
| 921021 | 0 | Light | + | 42.3 | 3.6 | Pass |
| | | yellowish | | | | |
| | | powder | | | | |
| | 1 | Same | + | 42.2 | 3.7 | Pass |
| | 3 | Same | + | 42.2 | 3.6 | Pass |
| | 5 | Same | + | 42.1 | 3.5 | Pass |
| | 10 | Same | + | 42.1 | 3.4 | Pass |

TABLE 14
Stability test in forceful light radiation of 4000 Lx

| | | | | | Weight | |
|---|---|---|---|---|---|---|
| | | | | | lost | |
| Batch | | Ap- | Identifi- | Amount of | after | Hygienical |
| No. | Days | pearance | cation | Paeoniflorin | drying | inspection |
| 921019 | 0 | Light | + | 42.4 | 4.1 | Pass |
| | | yellowish | | | | |
| | | powder | | | | |
| | 1 | Same | + | 42.4 | 4.1 | Pass |
| | 3 | Same | + | 42.3 | 4.0 | Pass |
| | 5 | Same | + | 42.1 | 4.8 | Pass |
| | 10 | Same | + | 42.2 | 4.7 | Pass |
| 921019 | 0 | Light | + | 44.3 | 4.3 | Pass |
| | | yellowish | | | | |
| | | powder | | | | |
| | 1 | Same | + | 44.3 | 4.3 | Pass |
| | 3 | Same | + | 44.2 | 4.2 | Pass |
| | 5 | Same | + | 44.1 | 4.1 | Pass |
| | 10 | Same | + | 44.2 | 4.0 | Pass |
| 921021 | 0 | Light | + | 43.2 | 4.0 | Pass |
| | | yellowish | | | | |
| | | powder | | | | |
| | 1 | Same | + | 43.2 | 4.0 | Pass |
| | 3 | Same | + | 43.3 | 4.0 | Pass |
| | 5 | Same | + | 43.4 | 3.8 | Pass |
| | 10 | Same | + | 43.3 | 3.6 | Pass |

Pharmacology

treatment of RA, TGP's effectiveness at anti-inflammation has been proven by animal experiments and clinical studies. The purpose of studies in this section is to determine whether TGP may influence the nerve, respiratory and cardiovascular systems in animals of different species and to provide data of references for clinical study and practice.

Materials and Method

1) Activity of Animals: 30 rats were randomly divided into three groups with the same number of rats, two TGP groups and one control group. Animals in the two TGP groups were given 50 mg or 100 mg/kg TGP, respectively, once a day for three days by gastric injection (i.g.), while the control group was given the same volume of physiological saline. A close observation and detailed records were made focused on the animals' general appearance, reaction to the surroundings, food consumption and other behavior.

Six dogs were divided into 2 groups. Dogs in group one were intravenously injected with 15 mg/kg TGP once daily for three days. Dogs in the other group were injected with physiological saline the same way. Activities of dogs in both groups were observed and recorded.

2) Sleep rhythm of rats: SD male rats were selected for the test. One week before the study started, the animals were implanted with mini electric poles in the patrol and occipital lobes of the brain, and temporal or neck muscles (bilateral). The operations were performed under anesthesia while the rat's head was fixed in a cranial stereotaxic frame, (Modal: J.W.−1). The implanted poles were well fastened to the skull.

All prepared rats were divided into two groups, 10 rats in each. 50 mg/kg of TGP was given to one group by gastric injection. The other group was given the same volume of physiological saline. Two hours after administration, rats were connected to a polygraph-monitoring instrument with recording device. The cortex electroencephalography and electromyography were recorded for 6 hours. The test was performed once daily for three days.

Test Results

TABLE 15
Effects of TGP in gastric perfusion: average
percentage in Sleep-wake Cycle (SWC) of rats

| | Dose | | Before | After Administration | |
|---|---|---|---|---|---|
| Group | (mg/kg) | SWC | Administration | 1 day | 3 days |
| Normal | 0 | Alert | 40.7 ± 4.9 | 41.3 ± 5.5 | 40.2 ± 5.9 |
| saline | | SWS | 52.0 ± 4.4 | 51.1 ± 5.4 | 52.6 ± 6.7 |
| | | PS | 7.3 ± 1.2 | 7.6 ± 1.7 | 7.2 ± 1.5 |
| TGP | 50 | Alert | 37.0 ± 3.2 | 36.4 ± 6.6 | 36.6 ± 7.1 |
| | | SWS | 54.8 ± 6.8 | 55.8 ± 6.8 | 56.2 ± 6.0 |
| | | PS | 8.2 ± 0.9 | 7.8 ± 1.8 | 7.1 ± 1.4 |

Cardiovascular System

The method and test materials applied in this section were in compliance with the Regulation for New Drug Application by Administration of Health and Sanitation, P.R. China. 1985:32. Preparation of TGP, solutions of designed concentrations, were given to the dogs orally and intravenously before the start of the test.

Dogs were intravenously anesthetized with pentobarbital sodium, 30 mg/kg. The heart rate, electrocardiograph and blood pressure were monitored and recorded. The blood pressure was measured with intubated sensors in the right carotid artery. The No. two limb connection was chosen for electrocardiographs record. The speed of recording paper was set at 50 mm/second, voltage 1 mv.

The study showed that TGP had no effect on the dogs' cardiovascular system, see Table 16 through Table 19

TABLE 16
Effects of TGP oral administration on dogs'
cardiovascular system (1)

| | | |
|---|---|---|
| Dose | Num. of | ECG |

| Group | (mg/kg) | dog | Before | 1 hr. after |
|---|---|---|---|---|
| Control | 0 | 3 | Normal | Normal |
| TGP | 10 | 3 | Normal | Normal |
| TGP | 280 | 3 | Normal | Normal |

TABLE 17
Effects of TGP oral administration on dogs'
cardiovascular system (2)

| | | Blood Pressure | | |
|---|---|---|---|---|
| | Heart Rate | (systolic/dia- | | |
| | (beat/min) | stolic) | | |
| Group | Before | 1 hr. after | Before | 1 hr. after |
| Control | 134.3 ± | 148.4 ± 27.6 | 139 ± 17.8/ | 140 ± 16/ |
| | 15.0 | | 98.5 ± 19 | 99 ± 15 |
| TGP | 139.0 ± | 144.0 ± 16.0 | 140 ± 20/ | 144 ± 15/ |
| | 17.0 | | 95 ± 15 | 97 ± 18 |
| TGP | 136.0 ± | 139.0 ± 20.0 | 145 ± 16/ | 143 ± 21.0/ |
| | 28.0 | | 97 ± 13 | 98 ± 28 |

Table 18
Effects of TGP intravenous injection on dogs'
cardiovascular system (1)

| | | | ECG | |
|---|---|---|---|---|
| Group | Dose (mg/kg) | Num. of dog | Before | 1 hr. after |
| Control | 0 | 3 | Normal | Normal |
| TGP | 15 | 3 | Normal | Normal |

TABLE 19
Effects of TGP intravenous injection on dogs'
cardiovascular system (2)

| | Heart Rate | Blood Pressure | | |
|---|---|---|---|---|
| | (beat/mm) | (systolic/diastolic) | | |
| Group | Before | 1 hr. after | Before | 1 hr. after |
| Control | 130.0 ± | 143.0 ± 30.0 | 143 ± 21/ | 150 ± 30/ |
| | 28.0 | | 97 ± 15 | 99 ± 36 |
| TGP | 135.0 ± | 140.0 ± 24.0 | 145 ± 17/ | 149 ± 20/ |
| | 35.0 | | 95 ± 28 | 96 ± 23 |

Influences on Respiratory System

The method and test materials applied in this section are in compliance with the Regulation for New Drug Application by Administration of health and sanitation, P.R. China. 1985:32. Preparation of TGP, solutions of designed concentrations, are given to the rabbits orally and intravenously before the start of the test.

Rabbits are monitored for their frequency and depth of respiration with Method of Dresser. Results show that no effect on the rabbits' respiratory system was detected. See Table 20 and Table 21.

TABLE 20
Effects of TGP gastric injection on respiratory
system of rabbit (1)

| Group | Dose (mg/kg) | Numbers of Animal |
|---|---|---|
| Control | 0 | 3 |
| TGP | 25 | 3 |

TABLE 21
Effects of TGP gastric injection on respiratory
system of rabbit (2)

| | | Depth of | | |
|---|---|---|---|---|
| | Respiratory | Respiration | | |
| | Rate (time/15 s) | (ml/15 s) | | |
| Group | Before | 1 hr. after | Before | 1 hr. after |
| Control | 15 ± 4 | 20 ± 6 | 190 ± 35.0 | 213 ± 27.5 |
| TGP | 16 ± 2 | 19 ± 3 | 186 ± 24.0 | 208 ± 30.0 |

TABLE 22
Effects of TGF intravenous injection on
respiratory system of rabbit (1)

| | Dose | |
|---|---|---|
| Group | (mg/kg) | Numbers of Animal |
| Control | 0 | 3 |
| TGP | 25 | 3 |

TABLE 23
Effects of TGP intravenous injection on
respiratory system of rabbit (2)

| | Respiratory | Depth of | | |
|---|---|---|---|---|
| | Rate | Respiration | | |
| | (time/15 s) | (ml/15 s) | | |
| Group | Before | 1 hr. after | Before | 1 hr. after |
| Control | 15 ± 4 | 20 ± 6 | 190 ± 35.0 | 213 ± 27.5 |
| TGP | 16 ± 2 | 19 ± 3 | 186 ± 24.0 | 208 ± 30.0 |

Acute Toxicity Studies

The TGP for this study was the product of Sanjiu Medical & Pharmaceutical Co., Ltd. In the clinical study conducted in P.R. China, the dosage that patients received was 2 capsules one time and 3 times a day. It is estimated at 30 mg/kg day. (The content on one capsule was 300 mg).

Materials

1) Drug: TGP was provided by laboratory of botanical chemistry, Pharmaceutical Institute of Anhui Medical University, China. The drug was dissolved in physiological saline right before use.

2) Animal: Kunming-strain mice, male and female, half to half, (18±2 grams) were provided by Animal Section of Anhui Medical University.

Method and Design

190 mice were randomly divided into three groups, each group was treated differently in drug administration: Intravenous injection (i.v.), Intraperitoneal injection (i.p.), and intragastric injection (i.g.). Each of the groups was re-divided into 6˜7 subgroups containing 10 mice each. The mice were observed, and notes were made regarding the toxic effects, abnormal symptoms and deaths for 7 days. The LD₅₀ were evaluated with the Reduced Probability Method.

The LD₅₀ of i.v. group is 159.56 mg/kg, probability ranges at 149.25-170.67 mg/kg. The LD₅₀ of i.p. group is 230.01 mg/kg, probability ranges at 185.56˜264.46 mg/kg. For the i.g. group, neither obvious reaction nor death could be observed when drug dosage was raised to more than 2500 mg/kg.

Result and Conclusion

The LD₅₀ of mice administered with TGP by way of i.v. and i.p. were 159.96 and 230.01 mg/kg, respectively. For oral administration, no negative reaction was found in the 7 days after large-dosage TGP (>2500 mg/kg). The conclusion is oral administration of TGP is safe in animal tests.

Chronic Toxicity Study

The experiment was intended to study the long-term toxic effects of TGP, to determine its dose response relationship and safe dosage.

Materials and Method

1) Drug: TGP was provided by laboratory of botanical chemistry, Pharmaceutical Institute of Anhui Medical University, China.

2) Animal: Sprage-Dawley rats, raised for six-weeks. Their weight range is 130±20 grams, half to half in sex. Hybrid dogs, 4˜6 months, weight 7.34±2.2 kg, half to half in sex.

3) Equipment: Platelet analyzer Model 100, USA Blood cell analyzer Model 70, USA TM Automatic biochemical analyzer

Aemstor, USA Electrocardiograph XOH-3 Shanghai, China

4) Study Design

Test on Rats

80 rats were divided into 4 groups at random, i.e., control, low, mid and high dose groups (TGP 0, 50, 1000, and 2000 mg/kg in dosage). Each contained an equal number of male and female rats.

The rats were brought to the laboratory and raised there one week before the start of the test. During this period, routine blood and urine tests were taken (twice). The same procedure was repeated at the end of the 90-day study.

The Drug was administered by intra-gastric injection, once a day in doses that are mentioned above. Pure water was given to the control group in the same volume.

The procedure within the 90 days included: observe and make notes on the toxic effects or any abnormal reactions everyday, and take their body weights once a week.

After 90 days, the rats were killed by bloodletting in carotid artery. Rat's blood was collected for biochemical assays. Organs in half of the rats were put in histopathological examination which includes the tissues of heart, liver, lung, pituitary gland, hypothalamus, spheres of brain, midbrain, medullary bulb, and spinal cord. All samples were taken from the same part of the organs of rats' spleen, kidney, stomach, intestine, ovary/testicle, bone marrow, thyroid, thymus, and adrenal gland.

Before microscope examination, tissues were fixed in neutral formalin and stained with hematoxylin-eosin in the routine paraffin section.

Test on Dogs

12 dogs were randomly divided into 3 groups. Two groups were fed with TGP in dosages of 280 mg/kg and 560 mg/kg daily, one serving as the control group. TGP was mixed in ground meat together with starch and a little bit salt, once a day in the morning. Dogs in the control group had the same meat paste without the drug. Food supply was sufficient after the morning feeding. Dogs were observed and recorded in the way the rats did except that an antiparasitic course, two feces exam and two electrocardiographs (ECG) were taken before the start of drug administration. After 90 days, the dogs were killed by bloodletting in carotid artery. Organs of the dogs were put in histopathological examination, which included the same tissues with rat test.

Results

Test in Rats

1) Four rats in the high dose group died during 51 to 61 days after successive TGP administration. It is indicated by autopsy that they died of bronchopneumonia.

2) The activity, feeding and weight gaining of rats in mid and low dose groups showed no significant difference with those in the control group.

3) The liver function test (SGPT) and renal function test (BUN) of all groups of rats were in the normal range. Yet the average of SGPT of rats in mid and low dose groups is obviously lower than those of the control group by the end of the 90 days.

4) For items of blood tests, all groups were in the normal range except that the number of platelet in the high dose group is significantly higher than those of the control group (P<0.01)

5) Histopathology: the listed eighteen tissues of rats in the three groups of administration showed no significant difference or special finding in compare with those in the control group. It is indicated that no toxic effect of TGP on those organs of rats.

TABLE 24
Weight gaining of rats in the 90-day test (1)

| | Number of | |
|---|---|---|
| Groups | animal | Weight before test (gram) |
| Control | 20 | 151.4 ± 32.9 |
| TGP 50 mg/kg | 20 | 136.1 ± 30.9* |
| TGP 1000 mg/kg | 20 | 153.6 ± 51.7* |
| TGP 2000 mg/kg | 16 | 142.5 ± 33.0* |

TABLE 25
Weight gaining of rats in the 90-day test (2)

| | Weight after | Net weight gain |
|---|---|---|
| Groups | test (gram) | (gram) |
| Control | 305.5 ± 82.3 | 154.6 ± 79.0 |
| TGP 50 mg/kg | 298.1 ± 72.3* | 161.9 ± 66.2* |
| TGP 1000 mg/kg | 285.7 ± 56.9* | 132.1 ± 42.8* |
| TGP 2000 mg/kg | 216.0 ± 42.9** | 76.5 ± 65.3** |

TABLE 26
Biochemical examination of rats in the 90-day test

| | Number of | | |
|---|---|---|---|
| Groups | animal | SGPT (unit) | BUN (mmol/L) |
| Control | 20 | 64.6 ± 10.0 | 6.35 ± 0.71 |
| TGP 50 mg/kg | 20 | 56.6 ± 6.6*** | 6.49 ± 1.32 |
| TGP 1000 mg/kg | 20 | 58.0 ± 9.4** | 6.35 ± 0.80 |
| TGP 2000 mg/kg | 16 | 68.3 ± 13.3* | 6.71 ± 0.99 |

TABLE 27
Blood tests in rats before and after the 90-day test

| Items\Groups | | Control | TGP 50 mg/kg | TGP 1000 mg/kg | TGP 2000 mg/kg |
|---|---|---|---|---|---|
| Hb | Before | 140 ± 11 | 135 ± 10 | 145 ± 28 | 150 ± 38 |
| (g/l) | After | 178 ± 20 | 177 ± 20 | 163 ± 20 | 170 ± 10 |
| RBC | Before | 6.72 ± 0.93 | 6.59 ± 1.00 | 6.98 ± 1.10 | 6.98 ± 0.96 |
| (10¹²/L) | After | 8.30 ± 1.00 | 8.80 ± 0.50 | 8.00 ± 1.00 | 8.00 ± 0.50 |
| MCV | Before | 58.6 ± 4.6 | 60.7 ± 3.3 | 62.0 ± 3.8 | 60.8 ± 3.2 |
| (fl) | After | 53.9 ± 3.0 | 54.0 ± 2.0 | 53.5 ± 2.0 | 55.3 ± 2.2 |
| MCH | Before | 21.6 ± 2.7 | 23.8 ± 3.6 | 22.4 ± 3.1 | 21.0 ± 4.4 |
| (pg) | After | 21.9 ± 1.6 | 20.4 ± 3.0 | 20.9 ± 2.0 | 21.3 ± 1.0 |
| MCHC | Before | 36.7 ± 4.5 | 36.6 ± 6.4 | 36.2 ± 4.8 | 33.6 ± 3.7 |
| (%) | After | 39.6 ± 2.0 | 39.2 ± 4.0 | 38.8 ± 3.0 | 38.4 ± 2.0 |
| HCT | Before | 0.38 ± 0.06 | 0.39 ± 0.45 | 0.40 ± 0.0 | 0.41 ± 0.06 |
| (L/L) | After | 0.43 ± 0.06 | 0.44 ± 0.05 | 0.43 ± 0.05 | 0.45 ± 0.04 |
| BPC | Before | 783 ± 93 | 800 ± 100 | 779 ± 92 | 775 ± 130 |
| (10⁹/L) | After | 770 ± 290 | 758 ± 270 | 897 ± 270 | 1140 ± 210** |
| WBC | Before | 23.6 ± 5.6 | 24.3 ± 5.4 | 23.8 ± 5.1 | 23.9 ± 6.7 |
| (10^{9/}L) | After | 21.0 ± 5.0 | 20.2 ± 4.0 | 22.8 ± 3.5 | 22.0 ± 4.0 |
| N (%) | Before | 20.2 ± 6.0 | 18.3 ± 6.0 | 18.5 ± 10 | 20.9 ± 1.4 |
| | After | 23.6 ± 5.1 | 19.8 ± 4.2 | 19.2 ± 5.4 | 19.8 ± 4.5 |
| L (%) | Before | 79.4 ± 4.8 | 79.9 ± 4. | 80.4 ± 5.6 | 79.5 ± 3.0 |
| | After | 73.2 ± 5.0 | 80.3 ± 8.0 | 79.5 ± 4.0 | 78.6 ± 8.5 |

Test in Dogs

1) Faeces of dogs in TGP groups appeared deep brown after drug administration, although the occult blood examinations were negative. In the first five days, different extent of loose stool could be observed in dogs of the high dose group, it was healed completely without any treatment.

2) One dog in the control group died of a general mycotic infection on the 63^{th} day. Another dog in the low dose group died of lobar pneumonia on the 84^{th} day.

3) No obvious difference in weight gaining between groups. There was no difference in food consumption between groups in the first stage of the test. But, dogs in TGP groups broke the condition to increase their food intake to a significant level after the 49^{th} day.

4) For items of blood tests, the platelet levels of dogs in TGP groups were much higher than those of the control group, they were also higher than those of themselves before drug administration. Both are of statistically significance (P<0.01). Other items of blood tests didn't show any difference or abnormity.

5) No obvious changes can be traced between groups for the appearance, behavior, activity, electrocardiograph and histopathological exams of dogs. It indicated that TGP had no toxic effect on heart, liver, kidney and all the eighteen organs of dogs during a long term study.

TABLE 28
Weight gaining in dogs in the 90-day test

| | | Weight | | Net |
|---|---|---|---|---|
| | Number of | before Test | Weight after | weight |
| Groups | animal | (kg) | Test (kg) | gain (kg) |
| Control | 3 | 7.17 ± 1.26 | 10.33 ± 2.75 | 3.17 ± |
| | | | | 1.53 |
| TGP | 3 | 7.00 ± 2.00 | 9.67 ± 3.53 | 2.67 ± |
| 280 mg/kg | | | | 1.61* |
| TGP | 4 | 7.88 ± 3.86 | 10.38 ± 2.32 | 2.50 ± |
| 560 mg/kg | | | | 1 47* |

TABLE 29
Biochemical examinations in rats in the 90-day test

| | Number | | | | |
|---|---|---|---|---|---|
| | of | SGPT (unit) | | BUN (mmol/L) | |
| Groups | animal | Before | After | Before | After |
| Control | 3 | 29.5 ± 0.5 | 28.0 ± 3.6 | 4.06 ± 0.99 | 3.70 ± |
| | | | | | 0.46 |
| TGP | 3 | 26.8 ± 3.0* | 21.3 ± 14.6* | 4.36 ± 0.14* | 2.28 ± |
| 280 mg/kg | | | | | 0.98* |
| TGP | 4 | 28.0 ± 1.2* | 35.2 ± 13.5* | 4.50 ± 0.71 | 3.38 ± |
| 560 mg/kg | | | | | 0.57* |

TABLE 30
Heart rate and ECG in dogs in the 90-day test

| | Number | | | ECG (II lead | |
|---|---|---|---|---|---|
| | of | Heart rate (beat/min) | | connection) | |
| Groups | animal | Before | After | Before | After |
| Control | 3 | 154.7 ± 39.88 | 182.84 ± 43.35 | normal | normal |
| TGP | 3 | 147.29 ± 30.55 | 174.65 ± 38.4 | normal | normal |
| 280 mg/kg | | | | | |
| TGP | 4 | 141.71 ± 19.61 | 155.65 ± 15.99 | normal | normal |
| 560 mg/kg | | | | | |

TABLE 31
Blood tests in dogs before and after the 90-day test

| | | | TGP | TGP |
|---|---|---|---|---|
| Items\ | Groups | Control | 280 mg/kg | 560 mg/kg |
| Hb | Before | 179 ± 9.9 | 174 ± 38 | 176 ± 27 |
| (g/l) | After | 183 ± 8.0 | 166 ± 37 | 168 ± 18 |
| RBC | Before | 5.86 ± 0.31 | 5.89 ± 0.28 | 6.07 ± 0.59 |
| (10¹²/L) | After | 6.96 ± 2.04 | 7.89 ± 2.28 | 8.27 ± 0.77 |
| MCV | Before | 87.0 ± 2.2 | 84.2 ± 7.1 | 82.6 ± 7.3 |
| (fl) | After | 76.6 ± 3.6 | 75.3 ± 3.5 | 78.0 ± 8.7 |
| MCH | Before | 30.8 ± 1.6 | 29.4 ± 2.9 | 29.3 ± 4.2 |
| (pg) | After | 27.7 ± 7.2 | 27.4 ± 1.9 | 29.1 ± 1.1 |
| MCHC | Before | 36.8 ± 1.9 | 37.7 ± 2.5 | 36.3 ± 4.0 |
| (%) | After | 39.8 ± 1.9 | 36.6 ± 2.3 | 38.3 ± 2.1 |
| HCT | Before | 0.50 ± 0.03 | 0.49 ± 0.03 | 0.49 ± 0.08 |
| (L/L) | After | 0.49 ± 0.07 | 0.51 ± 0.02 | 0.53 ± 0.04 |
| BPC | Before | 366 ± 192 | 363 ± 149 | 362 ± 152 |
| (10⁹/L) | After | 380 ± 183 | 711 ± 127 | 656 ± 108 |
| | Before | 22.1 ± 3.0 | 17.7 ± 2.7 | 21.0 ± 5.1. |
| (10⁹/L) | After | 18.6 ± 6.5 | 16.2 ± 39 | 18.1 ± 7.2 |
| N (%) | Before | 70 ± 1.5 | 65.6 ± 4.4 | 70.5 ± 3.3 |
| | After | 65 ± 10.1 | 61.0 ± 9.5 | 64.0 ± 4.6 |
| L (%) | Before | 27.5 ± 2.3 | 32.7 ± 4.0 | 28.4 ± 3.3 |
| | After | 32.3 ± 6.6 | 35.0 ± 9.5 | 32.3 ± 2.7 |

Conclusion

As an anti-inflammation agent and immunological regulator, TGP has been applied in human for clinical study. One single course of TGP treatment lasts 28 days. The period of chronic toxic test is determined at 90 days.

1) Animals in high dose groups showed symptom of loose stool in the first several days, yet those of lower dose did not. It indicated that the symptom is related to the high dose of TGP which is thought to have not been well absorbed in the intestine. In the same way, it is thought that a high dose TGP also disturbed the functional digestive system so that the group of animals were not as well in gaining weight as those of other groups.

2) By the end of the tests, Number of the blood platelet increased in animals of TGP dog groups and the high dose rat group (norm: 100˜600×10⁹/L). The mechanism is not clear

3) Faeces of animals of all groups appeared deep brown after drug administration, although the occult blood examinations were negative showing that no bleeding in digestive tract. A micro components of phenolic hydroxyl chemicals, like methyl-paeoniflorin in TGP, will combine with trivalent iron to have the violet reaction.

4) No traceable damages could be found in histopathological examinations of the eighteen main organs of animals after administration. It holds identical verdict with other results: the absolute normality on electrocardiograph, blood examination of SGPT and BUN. TGP has a very low toxic effect on the main organs of animals; neither did it cause any major problems of body function even with a much higher dosage in a proximately long time. It is harmless and of wide range of dose safety.

Test of Teratogenesis

Materials and Method

TGP was from the product of Sanjiu Medical & Pharmaceutical Co., Ltd. Before use, it was dissolved into distilled water for different concentration. The solutions were prepared for groups of different dosages of TGP, that is: high (2.16 g/kg), mid (360 mg/kg) and low (60 mg/kg). Ninety pregnant Wistar rats were numbered by their date of fertilization and randomly divided into five groups. The other two were for the control groups.

A positive and a negative control groups were also set up, rats were given acetosalicylic acid (250 mg/kg) in the positive group and distilled water of the same volume in the negative group.

From day 7 to 17 after pregnancy, the TGP and negative control groups were given solutions of drug and water as described above. The positive control group was treated with the acetosalicylic acid from day 9 to 11. Both were by gastric injection.

The body weight of rats in all groups was taken once every three days, and the amount of drugs were adjusted individually after the body weight. Rats were killed at day 20 of pregnancy, wombs and embryos were extracted by cesarean section, which was for the study and examination in a traditional way.

Results and Conclusion

1) Effects on body weight of mother rats: Body weight and weight increase of rats in high dose group were significantly lower than those of the negative control group (P<0.01 or P<0.05). But, it was not true for the mid and low dose groups (P>0.05).

TABLE 32
Effects on body weight gaining in pregnant rats

| | | | Body weight of pregnant | | | |
|---|---|---|---|---|---|---|
| | Dose | pregnancy/ | rats | | | weight |
| Group | (mg/kg) | fertility | Day 0 | Day 6 | Day 20 | increase |
| Control | 0 | 17/18 | 246.6 ± | 269.8 ± | 339.6 ± | 36.2 ± |
| A | | | 30.4 | 27.3 | 28.5 | 18.5 |
| Control | 250 | 17/18 | 246.9 ± | 268.9 ± | 331.0 ± | 33.9 ± |
| B | | | 42.2 | 42.5 | 40.2 | 13.6 |
| TGP | 2160 | 16/18 | 247.1 ± | 270.3 ± | 313.3 ± | 17.4 ± |
| | | | 29.7 | 26.6 | 30.5* | 8.7** |
| TGP | 360 | 16/18 | 246.8 ± | 270.8 ± | 343.7 ± | 41.9 ± |
| | | | 25.0 | 26.6 | 39.5 | 16.6 |
| TGP | 60 | 17/18 | 247.0 ± | 270.4 ± | 341.0 ± | 35.5 ± |
| | | | 23.1 | 19.7 | 24.2 | 16.6 |

2) Effects on the survival rate of embryos: As compared with the negative control, there was no significant difference among the TGP groups in average implantation, average litter survival rate, survival rate, absorptive embryo rate, and dead embryo rate (P>0.05). The acetosalicylic acid group had much higher absorptive embryo rate, and dead embryo rate, and lower survival rate (P<0.01).

TABLE 33
Effects on survival of rate embryos (1)

| | Dose | Num. of | Ave. Num. | live Embryo | |
|---|---|---|---|---|---|
| Group | (mg/kg) | Preg. | Implant | Num | % |
| N.Control | 0 | 17 | 9.9 | 162 | 94.2 |
| P.Control | 250 | 17 | 9.6 | 137 | 77.4** |
| TGP | 2160 | 16 | 9.1 | 158 | 89.8 |
| TGP | 360 | 16 | 9.2 | 160 | 92.5 |
| TGP | 60 | 17 | 9.9 | 156 | 91.2 |

TABLE 34
Effects on survival of rate embryos (2)

| | Absorptive | | Dead | | | |
|---|---|---|---|---|---|---|
| | Embryo | | Embryo | | Dead and Absorbed | |
| Group | Num | % | Num | % | Num | % |
| N.Control | 9 | 5.2 | 2 | 1.2 | 11 | 5.8 |
| P.Control | 27 | 15.2** | 13 | 7.3** | 40 | 22.6** |
| TGP | 15 | 8.5 | 3 | 1.7 | 18 | 10.2 |
| TGP | 11 | 6.4 | 2 | 1.2 | 13 | 7.5 |
| TGP | 13 | 7.6 | 2 | 1.2 | 15 | 8.8 |

3) Effects on growth of embryo rats: As compared with the negative control, there was no significant difference for the TGP groups in body weight of embryos, weight of the placentas, body and tail length of embryos. The acetosalicylic acid group had showed differences in the body weight and length of embryos (P<0.01 and P<0.05).

TABLE 35
Effects on placentas and growth of embryos (1)

| | | Number of live | Weight of Embryo |
|---|---|---|---|
| Group | Dose (mg/kg) | Embryo | (g) |
| N.Control | 0 | 162 | 358 ± 0.77 |
| P.Control | 250 | 137 | 2.79 ± 0.67** |
| TGP | 2160 | 158 | 3.15 ± 0.92 |
| TGP | 360 | 160 | 3.41 ± 0.59 |
| TGP | 60 | 156 | 3.49 ± 0.91 |

TABLE 36
Effects on placentas and growth of embryos (2)

| | Weight of | Body Length | Tail Length |
|---|---|---|---|
| Group | placentas (g) | (cm) | (cm) |
| N.Control | 0.63 ± 0.09 | 3.63 ± 0.30 | 1.25 ± 0.15 |
| P.Control | 0.55 ± 0.09* | 3.39 ± 0.20* | 1.23 ± 0.13 |
| TGP | 0.55 ± 0.16 | 3.45 ± 0.30 | 1.23 ± 0.10 |
| TGP | 0.58 ± 0.11 | 3.66 ± 0.33 | 1.28 ± 0.11 |
| TGP | 0.60 ± 0.14 | 3.65 ± 0.36 | 1.31 ± 0.13 |

4) Effects on the appearance of embryos and the development of organs and tissues: Rats in the TGP and negative control groups showed no obvious malformation in their appearance and internal organs or tissues. Yet, the acetosalicylic acid group appeared to have harelip, split tongue, short body and abnormal limbs in appearance (4.4%). The malformation rate of internal organs was 52.7% that was mainly hydrocephalus, hydronephrosis, bleeding internal organs and split tongue.

5) Effects on the development of skeletal system of embryo: As compared with the negative control, there was no significant difference in TGP groups of deformity rate in skeletal system (P>0.05). But, the acetosalicylic acid group appeared to have a much higher deformity rate in skeletal system (69.5%, P<0.01) which included multiple malformations: defect or delayed ossification in sternum and skull, defect or fusion of vertebra, deformation or fusion of ribs, and the ‘fourteen rib’.

TABLE 37
Effects on the development of skeletal system of
rat embryos

| | | |
|---|---|---|
| | | Embryo with |
| Dose | Embryo | abnormal skeleton |

| Group | (mg/kg) | examined | Number | % |
|---|---|---|---|---|
| N.Control | 0 | 93 | 21 | 22.6 |
| P.Control | 250 | 82 | 57 | 69.5 |
| TGP | 2160 | 97 | 24 | 24.7 |
| TGP | 360 | 98 | 21 | 21.4 |
| TGP | 60 | 90 | 14 | 15.6 |

TABLE 38
Abnormal skeletal system in rat embryos (1)

| | | | Defect/delayed | | Delayed | |
|---|---|---|---|---|---|---|
| | Abnormal | | ossification of | | ossification of | |
| | Skeleton | | the sternum | | the skull | |
| Group | Num. | % | Num. | % | Num. | % |
| N.Control | 21/93 | 22.6 | 21 | 22.6 | 5 | 5.4 |
| P.Control | 57/82 | 69.5** | 46 | 56.1** | 23 | 28.0** |
| TGP | 24/97 | 24.7 | 22 | 22.7 | 6 | 6.2 |
| TGP | 21/98 | 21.4 | 20 | 20.4 | 6 | 6.1 |
| TGP | 14/90 | 15.6 | 14 | 15.6 | 4 | 4.4 |

TABLE 39
Abnormal skeletal system in rat embryos (2)

| | Defect/fusion | | Deformation/ | | | |
|---|---|---|---|---|---|---|
| | of vertebra | | fusion of ribs | | Other deformations | |
| Group | Num. | % | Num. | % | Num. | % |
| N.Control | 0 | 0 | 0 | 0 | 0 | 0 |
| P.Control | 16 | 19.5** | 22 | 26.8** | 3 | 3.7 |
| TGP | 0 | 0 | 0 | 0 | 0 | 0 |
| TGP | 0 | 0 | 0 | 0 | 0 | 0 |
| TGP | 0 | 0 | 0 | 0 | 0 | 0 |

Based on the above results, test for the toxicity upon reproduction is in a high dosage of 2160 mg/kg, TGP showed no side effects on survival, appearance, organs and tissues development, and skeletal system development for the embryo. It is deducted that TGP does not have teratogenic action.

Mutagenic Test

Microbiologic Reverse Mutation Test (Ames' Test)

1) Materials and Method:

The standard bacteria strains histidine-requiring salmonell typhimurrium mutants TA 97, TA 98, Ta 100 and TA 102 TGP was dissolved in double distilled water to make it at five concentrations, i.e. 10000 μg, 1000 μg, 1001 μg, 10 μg, and 1 μg for each plate.

Mixed with TGP in half of the plates, a standard metabolic activation system S₉ was applied. That was a parallel test between two groups of plates with or without the mixture of S₉.

Mitomycin C

Each course was to be performed twice for three plates of different concentrations. Positive indicators and solvent control were included within all courses. Courses were performed with a ‘pre-incubation’ method, and the final result comes out in average from total six values.

TABLE 40
Mcrobiologic reverse mutation test of TGP (S₉₋)

| | Concentration | | | | |
|---|---|---|---|---|---|
| Drugs (−S₉) | μg/plate | AT 97 | AT 98 | AT 100 | AT 102 |
| | 0 | 128 ± 15.8 | 27 ± 8.6 | 137 ± 31.3 | 214 ± 37.0 |
| DKS | 50 | 1262 ± 38.9 | 528 ± 27.1 | 1072 ± 40.5 | >2000 |
| TGP | 1 | 127 ± 26.0 | 23 ± 11.3 | 106 ± 13.3 | 228 ± 44.0 |
| TGP | 10 | 123 ± 14.0 | 20 ± 6.9 | 117 ± 28.1 | 232 ± 38.0 |
| TGP | 100 | 148 ± 23.0 | 23 ± 5.9 | 140 ± 11.1 | 197 ± 38.2 |
| TGP | 1000 | 141 ± 16.2 | 26 ± 4.4 | 129 ± 18.5 | 181 ± 21.0 |
| TGP | 10000 | 10 26 ± 6.7 | 10 ± 0.9 | 143 ± 23.0 | 165 ± 17.0 |

TABLE 41
Mcrobiologic reverse mutation test of TGP (S₉₊)

| Drugs | Concentration | | | | |
|---|---|---|---|---|---|
| (+S₉) | μg/plate | AT 97 | AT 98 | AT 100 | AT 102 |
| | 0 | 159 ± 20.6 | 33 ± 6.8 | 118 ± 30.8 | 242 ± 41.0 |
| 2-AF | 10 μg | 598 ± 27.4 | 974 ± 30.7 | 772 ± 29.8 | >7200 (MMC 2 μg/plate) |
| TGP | 1 | 166 ± 30.3 | 26 ± 5.6 | 122 ± 27.4 | 265 ± 35.4 |
| TGP | 10 | 153 ± 15.3 | 35 ± 10.9 | 132 ± 43.1 | 312 ± 38.4 |
| TGP | 100 | 154 ± 22.2 | 28 ± 7.7 | 115 ± 13.6 | 247 ± 35.7 |
| TGP | 1000 | 166 ± 22.8 | 17 ± 6.1 | 130 ± 14.4 | 244 ± 46.8 |
| TGP | 10000 | 128 ± 25.7 | 17 ± 9.2 | 125 ± 24.6 | 183 ± 59.8 |

2) Results

The ratio for numbers of reverting colonies of positive control groups over negative (Rt/Rc) is larger than 3. Either in the presence or absence of S9 metabolic activation, the number of reverting colonies in groups treated with TGP did not exceed 2 fold of the number of their corresponding spontaneous reverting colonies in any histidine requiring salmonella thphimurium indicator strain TA 97, TA 98, Ta 100 and TA 102.

Therefore, TGP was unable to induce gene mutation in these strains and the Ames test result was negative.

Micronucleus Test

1) Materials and Method:

ICR mice of sexual maturity, all males. Six random groups with eight mice in each one.

TGP was dissolved in double distilled water before use. With a gradient of 4:1, the four concentrations were set at 2500 mg/kg, 625 mg/kg, 126 mg/kg and 39 mg/kg. The top dose was at 2500 mg/kg, since it was demonstrated in another experiment that no death in gastric injection even in this accelerated dose.

Cytoxan (CTX)

The drug was administered by gastric injection at the above doses together with the positive indicator (Cytoxan) and the solvent control. The course was repeated after 24 hours and animals were killed six hours after the second administration. Samples were taken and examined.

TABLE 42
Result of TGP's micronucleus test in mice

| | Dose | Num. of | Num. of | | p |
|---|---|---|---|---|---|
| Group | (mg/kg) | mice | PCE | MPCE % ± SD | value |
| Solvent | 0 | 8 | 8000 | 1.37 ± 1.40 | |
| CTX | 80 | 8 | 8000 | 41.37 ± 10.29 | <0.01 |
| TGP | 39 | 8 | 8000 | 1.40 ± 1.00 | >0.05 |
| TGP | 156 | 8 | 8000 | 1.75 ± 0.82 | >0.05 |
| TGP | 625 | 8 | 8000 | 1.50 ± 0.70 | >0.05 |
| TGF | 2500 | 8 | 8000 | 1.50 ± 1.22 | >0.05 |

2) Results

The effects of TGP on micronuclei are shown in the above table. It was found that TGP did not produce any statistically significant increase in numbers of micronucleated polychromatic erythrocyte compared with the negative control. TGP is negative in this test and was considered not clastogenic.

Chromosome Aberration Test

1) Materials and Method:

TGP, was dissolved in methyl-sulfoxide. 333.3 μg/ml, 111.1 μg/ml, 37.0 μg/ml, and 12.3 μg/ml in concentrations. Cyclophosphamide (CP): 60.0 μg/ml in methyl-sulfoxide Mitomycin C (MMC): 0.2 μg/ml in methyl-sulfoxide Chinese hamaster lung fibroblast (CHL) cells

In the Fifty Percent Growth Inhibition Test of TGP, IC₅₀ of TGP was 111.1 μg/ml. It was 333.3 μg/ml with the standard metabolic activation system S₉ Take both IC₅₀ as the top doses and 3:1 for gradient, two sets of 3 dose groups were set up respectively for S₉− and S₉+. They were 111.1 μg/ml, 37.0 μg/ml, 12.3 μg/ml groups and 333.3 μg/ml, 111.1 μg/ml, and 37.0 μg/ml groups.

With method of Ishidate (1981), cells were collected in 24 and 48 hours for S₉− groups and 24 hours for S₉+. All assays were performed twice and their average was taken for final results.

TABLE 43
Result of TGP's chromosome aberration test (1)

| | | |
|---|---|---|
| Dose | Time of | S₉₋ |

| Group | (μg/ml) | Incubation | Deformation | Rate (%) |
|---|---|---|---|---|
| Solvent | 0 | 24 | g, p, b | 1.5 |
| MMC | 0.2 | 24 | g, b, p, t, f | 79.5 |
| CP | 60.0 | 24 | | |
| TGP | 12.3 | 24 | g, b | 2.0 |
| TGP | 37.0 | 24 | g, p | 2.0 |
| TGP | 111.1 | 24 | g, b, p | 3.5 |
| TGP | 333.3 | 24 | | |
| Solvent | 0 | 48 | g, b | 2.0 |
| CP | 15 | 48 | g, b, p, r, t, | 34.5 |
| | | | f | |
| TGP | 12 | 48 | g, b | 2.5 |
| TGP | 37 | 48 | g, b | 3.0 |
| TGP | 111 | 48 | g, b, p | 4.0 |

TABLE 44
Result of TGP's chromosome aberration test (2)

| | S₉₊ | | |
|---|---|---|---|
| Group | Deformation | Rate (%) | Result |
| Solvent | g, b | 2.0 | — |
| MMC | | | |
| CP | g, p, b, r, t, f | 44.0 | + + |
| TGP | | | — |
| TGP | g, b | 2.0 | — |
| TGP | g, b, p | 2.5 | — |
| TGP | g, b, p | 2.5 | — |
| Solvent | | | — |
| CP | | | + + |
| TGP | | | — |
| TGP | | | — |
| TGP | | | — |

2) Results

TGP did not induce any significant chromosome aberration in CHL cells

Pharmacodynamics

Experimental materials

Animals

Sprage-Dawley (SD) rats, male, 2˜3 months in age, weight 180±30 g

C₅₇BL/6J mice, male, 6˜8 weeks in age, weight 20±3 g

ICR mice, male and female, 8˜12 weeks in age, weight 31±3 g Above animals were provided by Experiment Animal Section, Anhui Medical University, Institute of Clinical Pharmacology

BALB/C mice, male, 12˜16 weeks in age, weight 24±3 g, provided by Experiment Animal Section, Anhui Medical Research Institute

Cells and Clones

L929 cell strain (mouse fibroma cell) product of Immunity Department, Academy of Medical Science, Shandong.

Hep₂ Cell (interferon detective cell) presented by Institute of Virology, Wuhan.

Reagents and Drugs

Total Glycosides of Paeony (TGP) was provided by Department of Phytochemistry, Anhui Medical University, Institute of Clinical Pharmacology. The powder drug was to be dissolved evenly in normal saline or normal nutrient agent right before administration.

Indomethacin (IM) and Cyclophosphamide (Cy), products of Twelfths Pharmaceutical Company, Shanghai

Lobenzarit disodium; CCA, Japanese products

BCG vaccine, product of Biological Product Institute, Shanghai

Concanavalin A (conA), Lipopolysaccharide (LPS), Actinomycin D, Car-xi macin (A23187), Zymosan, Collagenase Type II (440 U/mg), Arachidonic acid (AA) and Prostaglandin B2 (PGB₂) are all products by Sigma Company.

Rehabilitated human Tumor Necrosis Factor a (rhTNFa, 4×10⁷ U/mg protein), a product of BASF/Knoll Company.

MTT and Scopoletin, products of Fluka Company

Trypsin (1:250), product of Difco Company

Dinitrofluorobenzene (DNFB), product of Xingta Chemical Firm, Jinshan, Shanghai.

Horse radish Peroxidase (HRPO), Biological Institute, Shanghai, Chinese Academy of Science.

Thioglycollate (TG), Biological Products Institute, Beijing

New town fowl pest Virus (NDV), Anhui Biological Medicine Laboratory

Fluorandiol Isothiocyanic acid sheep IgG against mice, from Dr. Yigong Ge, Changzheng Hospital, Shanghai

OX series of mono-clone antibody against rat T cells subgroups, from Dr. Mason, Immunology Center of Oxford University, England

Leukotriene B₄, (LTB₄), present from Prof. Tianli Yue, 2^{nd} Military Medical Collage

Culture medium PRMI1640 and DMEM, products of Gibico Company

[³H] TdR (666 TBq/mmol), product of Atomic Energetic Institute, China

[³H] PGE₂ Radio-immunity Test Kit (5.92 TBq/mmol), Pharmacological Lab, Institute of Basic Medicine, Chinese Academy of Medical Science

Instruments

CO₂ Incubation cabinet, product of Chongqing Experiment Instrument Factory

FJ-2107 liquid scintillation counter, product of 262 Factory, Xian

DYQ-III multi-duct cell collector, product of Shaoxing Potang Medical Equipment Factory

DG-3021 enzyme immunoassay detector, product of Nanjing Donghua Electronic Company

MK-500 volume measuring instrument, Hitach-650 fluorospectrophotometer, and LC-6A HPLC are Japanese products

Test on Animals with Inflammations

Effects on Adjuvant Arthritis in Rats

1) The preparation of Freund's Complete Adjuvant (FCA) and the Animal Models

Inactive the BCG vaccine under temperature 80° C. for 1 hour, added it to the sterile fluid wax and shook to make the mixture an emulsion of 10 mg/ml—Freund's Complete Adjuvant (FCA). Injected 0.1 ml FCA intracutaneously on do to the left hind foot of the rats. The volumes of the feet both injected and non-injected were measured, which represent primary and secondary inflammations. The volume changes before and after injection made up the “degree of swelling”.

2) Groups and Administration

Six groups of rats, each contained six. One for normal control that receives no injection and no treatment; one for positive control of adjuvant arthritis that were injected but were not treated. The other four groups were injected on d0 and administered with TGP and CCA by gastric infusion from d12 through d20. TGP were administered in three different doses and CCA in one dose.

3) Results

As result, groups of all three doses of TGP and CCA show certain improvements in joint swelling and the weight of their thymus are gained, some are even significantly changed. A study in general pathology are taken within the test. It shows that TGP result in the decreasing in fibrinous exudates, inflammatory cell infiltration, and hyperplasia of joint synovium. It also shows that TGP may increase the quantity of lymph cells located in the cortex or medulla of the thymus.

The degree of swelling on the non-injected feet (model of secondary inflammation) is showed in Table 45. The degree of swelling on the injected feet (model of primary inflammation) is showed in Table 46.

TABLE 45
Effect of TGP on secondary adjuvant arthritis (non—injected foot)

| | Dosage | Degree of Swelling (ml) | | | | | Weight of |
|---|---|---|---|---|---|---|---|
| Groups | (mg/kgd) | d12 | d16 | d20 | d24 | d28 | Thymus |
| Norm | — | — | — | — | — | — | 9.0 ± 2.9 |
| +Ctrl | — | 0.40 ± 0.10 | 0.55 ± 0.15 | 0.75 ± 0.27 | 0.59 ± 0.30 | 0.83 ± 0.24 | 9.8 ± 2.3 |
| TGP | 10 | 0 35 ± 0.08 | 0 43 ± 0.14 | 0.33 ± 0.14** | 0.24 ± 0.14** | 0.27 ± 0.15** | 16.2 ± 3.4** |
| | 50 | 0.40 ± 0.09 | 0.36 ± 0.12 | 0.19 ± 0.06** | 0.14 ± 0.08** | 0.24 ± 0.09** | 19.3 ± 5.9** |
| | 100 | 0.39 ± 0.12 | 0.36 ± 0.150 | 0.29 ± 0.07** | 0.26 ± 0.07** | 0.24 ± 0.15** | 13.8 ± 2.5** |
| CCA | 50 | 0 40 ± 0.07 | 0 54 ± 0.23 | 0.41 ± 0.22* | 0.14 ± 0.15** | 0.05 ± 0.02** | 14.3 ± 3.9** |

d0 FCA Injection; d12˜22 Administration of TSP or CCA etc.; Mean ± SD, n = 6; * P < 0.05, ** P < 0.01 in comparison with “+Ctrl” group

TABLE 46
Effect of TGP on primary adjuvant arthritis (injected foot)

| Groups in | Dose | Degree of swelling | | | |
|---|---|---|---|---|---|
| treatment | (mg/kgd) | d1 | d3 | d5 | d7 |
| Negative | — | 0.63 ± 0.19 | 1.17 ± 0.03 | 0.88 + 0.24 | 0.58 ± 0.08 |
| TGP | 50 | 0.59 ± 0.21 | 0.82 ± 0.19** | 0.52 ± 0.15** | 0.42 ± 0.17* |
| TM | 2 | 0.53 ± 0.23 | 0.58 ± 0.12** | 0.36 ± 0.09** | 0.31 ± 0.09** |
| CCA | 50 | 0.66 ± 3.24 | 0.94 ± 0.17 | 0.67 ± 0.18 | 0.63 ± 0.18 |

D0 FCA Injection; d3˜d6 Administration of TGP, IM and CCA; Mean ± SD, n = 6; *P < 0.05, **P < 0.01 in comparison with control group

4) TGP's Effects on Certain Inflammation Related Cell Activities

A serial of cell activities in connection with inflammation are also examined during the above tests: the amount of H₂O₂, IL-1, and PGE2 which are produced by the macrophage of abdominal cavity, the Con A proliferative reaction of thymocyte, the Con A induced IL-2 creation of splenocyte, the amount of sub-grouped T cells in the peripheral circulation, and the IL-1, TNF, and PGE₂ generated by synovial cells, etc. (See FIG. 1 through FIG. 4)

Effects on Carrageenin Injection Induced Foot Swelling

1) Preparation of the Animals

Inject 0.1 ml of 1% Carrageenin subcutaneously to the right hind foot of rat, measure the girth of ankle joint accurately with a special narrow ruler. The difference of the girth of the ankle joint before and after injection makes up the “degree of swelling”.

2) Group and Administration

40 rats were randomly divided into 4 groups: one for the control group, the others three were TGP 60 mg/kg, TGP 120 mg/kg, and IM 10 mg/kg groups. Drugs and plain solvent were given 30 minutes before Carrageenin injection. The result indicated in FIG. 5 shows that both doses of TGP were effective in inhabiting the swelling. (See FIG. 5)

Effects on Weakened Delayed Type Hypersensitivity (DTH) Reaction of Mice Induced by Cyclophosphamide

ICP mice are randomly divided into 8 groups, seven mice in each. All groups are to be tested for DTH reaction yet were treated differently in drug administration:

Group one is a solvent control group in which, neither cyclophosphamide nor TGP are given; group two is TGP control group; group 3, 4, and 5 are for the wakened DTH models treated with three doses of cyclophosphamide; group 6, 7, and 8 are wakened DTH groups with the interference of TGP. Doses of TGP, when applied, are administered 5 mg/kg per day intraperitoneally. It starts in 6 hours right after the first sensitization and last for five days. Results are indicated in FIG. 6.

Effects on Enhanced Delayed Type Hypersensitivity (DTH) Reaction of Mice Induced by Cyclophosphamide

ICR mice were divided randomly into three groups: one solvent control, one cyclophosphamide control, and one cyclophosphamide plus TGP group (see table 47). Cyclophosphamide was administered 3 days before (d-3) the first day sensitization (enhanced DTH model). TGP was administered in d-3 through d4, 5 mg/kg day. The results are shown in Table 5.3. TGP if effective against the enhancement in DTH reaction.

TABLE 47
Effect on enhanced DTH reaction by cyclophosphamide in mice.

| | | Number of | Degree of ear |
|---|---|---|---|
| Groups | Dose (mg/kg) | animal | swelling |
| Solvent | — | 9 | 19.6 ± 6.1 |
| (control) | | | |
| Cy + Solvent | 250 | 8 | 28.1 ± 7.2** |
| Cy + TGP | 250, 5 | 10 | 20.1 ± 5.8^{Δ} |

Cy is administered i.p. at d-3, TGP or solvent is administered i.p. d-3˜d4. Mean ± SD. ⁶⁶ P < 0.05, in comparison with Cy control; **P < 0.01, in comparison with solvent control

Effects on Weakened IgM Formation of Mice Induced by Cyclophosphamide

Animals were randomly divided into four groups: solvent control group, TGP control group, Cy group, and Cy plus TGP group. All groups of mice were sensitized with 0.2 ml 10% SRBC suspension i.p., mice were also administered 125 mg/kg of cyclophosphamide 3 days before the sensitization to make up the models of weakened immunoreactions. TGP was administered 5 mg/kg per day for 4 days, it started in 6 hours after the first sensitization. The mice were killed on the fifth day, organs of spleens and thymus were weighted and quantity of IgM in the spleen cells examined.

The result showed that TGP has the antagonistic effects upon IgM formation in the spleen cells of mice, and inhabit the decrease of spleen and thymus weights in the mice with weakened immunoreactions. Yet it showed few effect upon normal mice.

TABLE 48
Effect of TGP on weakened IgM formation mice by cyclophosphamide.

| | | Quantity | | |
|---|---|---|---|---|
| | Dose | of Ig M | Weight of | Weight of |
| Groups | (mg/kg/day) | (A value) | Spleen | thymus |
| Solvent | — | 0.59 ± 0.14 | 68.3 ± 22.7 | 12.2 ± 3.9 |
| (control) | | | | |
| TGP | 5 × 4d | 0.61 ± 0.14 | 59.8 +8.2 | 12.8 ± 4.2 |
| Cy + solvent | — | 0.41 ± 0.10* | 56.6 +11.5 | 6.2 ± 4.7* |
| Cy + TGP | 5 × 4d | 0.57 ± 0.06^{ΔΔ} | 63.3 ± 14.4 | 9.3 ± 3.8 |

Cy is administered 125 mg/kg i.p. at d-3; Mean ± SD, n-6; *P < 0.05, in comparison with solvent control; ^{ΔΔ}P < 0.01, in comparison with Cy control.

Effects on Enhanced IgM Formation of Mice Induced by Cyclophosphamide

See table 49 and table 50 for grouping and test procedures: spleen cells of donor mice are transferred to acceptor mice. And, SRBC (4×108/mouse i.p.) are given to the acceptor mice for additional immune enhancement. Acceptors are killed on the fifth day of transference and examined for the quantity of IgM in the spleen cells.

TABLE 49
Effect of TGP on enhanced Ig M formation mice by cyclophosphamide (1)

| | Donor mice | | |
|---|---|---|---|
| | Immune or | Doses | Time of |
| Groups | treatment | (mg/kg d) | administration |
| A | — | — | — |
| B | SOI + solvent | — | — |
| C | SOI + Cy^{b} + solvent | — | — |
| D | SOI + Cy + TGP | 5 | d1˜d8 |

TABLE 50
Effect of TGP on enhanced Ig M formation mice by
cyclophosphamide (2)

| | Acceptor mice | | |
|---|---|---|---|
| | Number of | | |
| Groups | animal | Ig M (A) | Rate of inhibition |
| A | 8 | 0.70 ± 0.09 | — |
| B | 9 | 0.38 ± 0.03** | 45.7 |
| C | 7 | 0.53 ± 0.03^{ΔΔ} | 24.3 |
| D | 7 | 0.35 ± 0.06^{##} | 50.0 |

Donor mice n = 5; Cy 100 mg/kg i.p. 2^{nd} day after sensitization; Mean ± SD; **P < 0.01, in comparison with A group; ^{ΔΔP < 0.01, in comparison with B group;} ^{##P <0.001, in comparison with C group.}

The results from test 3˜6 indicate that TGP is a dual antagonist, it acts as an enhancer or moderator in the weakened or enhanced function of cellular and humoral immunity induced by cyclophosphamide.

Test on Main Immunocytes Functions

TGP interferes activities of the three major immuncytes in their immune functions in proliferation and secretions. Its effect is dose-dependent.

Effect on Functional T Lymph Cells

1) Effect on T Cell Induced Mitogen Reaction

With the exist of Cona (3 μg/ml), TGP will act on the proliferating reaction of spleen lymph cell in C₅₇ B3L/6J mice. FIG. 7 shows that when TGP is in the concentrations of 0.1˜1.63 μg/ml, it will increase the reaction. The dose-effect curve is in the shape of a bell that indicates a dose-dependent dual effect.

TABLE 51
Effect on MDV induced increasing α-IFN titer

| | Concentration of TGP (μg/ml) | | | | | Virus | TGP |
|---|---|---|---|---|---|---|---|
| | 1000 | 100 | 10 | 1 | 0.1 | Control | Control |
| EXP | 10.04 | 13.29 | 13.62 | 12.51 | 12.29 | 12.07 | <4 |
| 1 | | | | | | | |
| EXP | 11.95 | 13.62 | 13.95 | 12.62 | 12.84 | 12.37 | <4 |
| 2 | | | | | | | |
| EXP | 11.89 | 12.51 | 13.25 | 12.69 | 12.77 | 12.19 | <4 |
| 3 | | | | | | | |
| Mean | 11.29 ± | 13.14 ± | 13.61 ± | 12.61± | 12.63 ± | 12.21 ± | <4 |
| ± | 1.09 | 0.57 | 0.35 | 0.09 | 0.30 | 0.15 | 0 |
| SD | | | | | | | |

TABLE 52
Effect on ConA (10 μg/ml) induced γ-IFN titer

| | Concentration of TGP (gg/ml) | | | | | ConA | TGP |
|---|---|---|---|---|---|---|---|
| | 1000 | 100 | 10 | 1 | 0.1 | Control | Control |
| EXP | 9.52 | 10.35 | 11.09 | 11.02 | 10.52 | 9.78 | <3 |
| 1 | | | | | | | |
| EXP | 9.32 | 9.76 | 10.19 | 9.58 | 9.52 | 9.02 | <3 |
| 2 | | | | | | | |
| EXP | 9.24 | 9.74 | 10.52 | 10.74 | 10.74 | 9.24 | <3 |
| 3 | | | | | | | |
| EXP | 8.20 | 8.16 | 8.87 | 9.54 | 8.45 | 7.89 | <3 |
| 4 | | | | | | | |
| EXP | 8.85 | 10.16 | 10.98 | 11.23 | 9.98 | 9.73 | <3 |
| 5 | | | | | | | |
| Mean | 9.03 ± | 9.63 ± | 10.33 ± | 10.42 ± | 9.84 ± | 9.13 ± | <3 |
| ± | | | | | | | |
| SD | 0.52 | 0.86 | 0.89 | 0.81 | 0.91 | 0.77 | 0 |

Effect on Lipopolysaccharide (LPS) Induced B lymph Cell Proliferation

FIG. 9 indicates that TGP with 8˜1000 ng/ml in concentration will increase the LPS (61 μg/ml) induced spleen lymph cell proliferation in mice. The dose-effect curve indicates that it is a dose-dependent dual effect.

Effect on Functional Macrophage

1) Effect on LPS Induced Celiac Macrophage Interleukin-1 (IL-1) Production in Rats

TGP with concentration of 0.5˜125 μg/ml may increase the LPS (6μg/ml) induced celiac macrophage's production of IL-1 in rat. The dose-effect curve indicates that it is a dose-dependent dual effect (FIG. 10).

2) Effect on LPS Induced Celiac Macrophage Production of Tumor Necrosis Factor (TNF)

TGP with concentration of 0.5˜125 μg/ml may increase the LPS (5 μg/ml) induced celiac macrophage's production of TNF in rat. The dose-effect curve indicates that it is a dose-dependent dual effect (Table 53).

TABLE 53
Effect on LPS induced TNF production

| TGP (μg/ml) | LPS (μg/ml) | TNF (U/ml) |
|---|---|---|
| 0 | 5 | 138.2 ± 15.1 |
| 0.5 | 5 | 180.6 ± 19.4* |
| 2.5 | 5 | 230.7 ± 24.6** |
| 12.5 | 5 | 252.6 ± 28.4** |
| 62.5 | 5 | 224.2 ± 20.7** |
| 125 | 5 | 182.4 ± 20.2* |
| 250 | 5 | 115.8 ± 14.6 |

Mean ± SD, n = 5;
*P < 0.05,
**P < 0.01, in comparison with LPS control group.

3) Effect on Zymosan Induced Celiac Macrophage H₂O₂ Production

TGP in concentration of 0.45˜62.5 μg/ml may obviously increase celiac macrophage's H₂O₂ release in rat. The dose-effect curve indicates that it is a dose-dependent dual effect (FIG. 11)

4) Effect on A23187 Induced Celiac Macrophage's Production of PGE₂

In a solution of A23187 (0.1 μmol/L), TGP in concentration of 0.1˜10 μg/ml may increase celiac macrophage's production of PGE₂ in rat. The dose-effect curve indicates that it is a dose-dependent dual effect (Table 53). In A23187 of 1 μmol/L solution, TGP will inhabit this reaction and it is also dose-dependent.

TABLE 54
Effect on A23187 induced PGE₂ by celiac macrophages in rat

| | | PGE₂ (pg/10⁶ |
|---|---|---|
| TGP (μg/ml) | A 23187 (μmol/L) | Cells) |
| 0 | 0.1 | 1045 ± 105 |
| 0.01 | 0.1 | 1080 ± 70 |
| 0.1 | 0.1 | 1590 ± 194.5* |
| 1 | 0.1 | 2650 ± 440.5** |
| 10 | 0.1 | 1327 ± 380* |
| 100 | 0.1 | 1035 ± 169 |
| 0 | 1 | 1456.5 ± 184 |
| 0.01 | 1 | 1245.5 ± 78.5* |
| 0.1 | 1 | 962 ± 48* |
| 1 | 1 | 863 ± 42* |
| 10 | 1 | 806.5 ± 335* |
| 100 | 1 | 672.5 ± 445** |

Mean ± SD, n = 4;
*P < 0.05,
**P < 0.01, in comparison with control group (TGP = 0).

5) Effect on A23187 Induced Celiac Macrophage's Leukotriene B4 (LTB₄) Production

FIG. 12 indicates that In A23187 of 1 μmol/L solution, TGP (0.01˜100 μg/ml) will inhabit A23187 (1 μmol/L) induced LTB₄ generation by celiac macrophage in rat and this reaction is dose-dependent (FIG. 12)

Phase II Clinical Study

As an anti-inflammatory and immune regulatory drug showing positive effects in the pre-clinical studies in vitro and in vivo, Total Glycosidess of Paeony claims to have an outstandingly effective cure against Rheumatoid Arthritis. In comparison with the second stage drugs currently used, TGP is also unique for its natural resources and its producing procedures. It is extracted from a natural botanical material, which has been approved by experiments and experience through a long period that it has lower toxic effect upon animals and human body.

To determine the effectiveness and safety of TGP, Clinical Center of Pharmacology, Second Medical University, Shanghai was authorized by Department of Public Health, P. R. China in conducting the clinical trial Phase II in patients with rheumatoid arthritis. It was a multiple-centered, randomized, parallel, positive controlled Phrase II clinical study.

The study was conducted in five hospitals among 450 patients of rheumatoid arthritis, 300 were administered with TGP and 150 with Methotrexate (MTX)—an effective second-line remedy for rheumatoid arthritis that was selected to be the drug for positive control.

Out of the 450 patients in 5 hospitals, 120 cases (60 patients for TGP and 60 patients for MTX) were studied double-blinded; and another 120 cases among single blinded study were designed for an extra 12 weeks long-term observation. All clinical studies except the above mentioned 120 cases were conducted in a single-blind condition.

Among 450 patients in 5 hospitals, two doses of TGP were set up for the effect test. 60 patients in Gulou hospital accepted low dose TGP treatment while the others were tested with a high dose TGP. MTX positive controls were set up for every hospital.

TABLE 55
Numbers of patients in the five center

| | Cases | | Dose | | |
|---|---|---|---|---|---|
| Center | TGP | MTX | set | Test design | Long term |
| Renji Hosp., | 30 | 30 | High | Double blind | 6 months |
| Shanghai | | | | | |
| Provincial | 30 | 30 | High | Double blind | 6 months |
| Hosp., Anhui | 30 | 30 | High | Single blind | |
| Gulou Hosp., | 60 | 30 | Low | Single blind | |
| Nanj ing | | | | | |
| Guanghua Hosp., | 60 | 30 | High | Single blind | |
| Shanghai | | | | | |
| Attached Hosp. | 60 | 30 | High | Single blind | |
| of S.M.U | | | | | |

Patient Selection

Criteria in Patient Selection

1) Age 18˜65, male or female who agreed to the investigational treatment for 12 weeks to 6 months. History of rheumatoid arthritis was within 3 years.

2) Documented Rheumatoid Arthritis based on a diagnostic criteria of 1987 Revised ARA Criteria for the Classification of Rheumatoid Arthritis

3) Diagnosis of a typical active stage of RA: A patient is said to be on an active stage of RA if he or she has satisfied at least 4 of the following 5 items.

Pain at rest: Pain at minimum of moderate intensity in joints at rest.

Morning stiffness: Morning stiffness, lasting at least 1 hour before maximal improvement.

Swelling of joints: At least 3 hours of area swollen in joint(s) in a day.

Tenderness of joints: Tenderness of joint that involves at minimum of 8 joints.

Erythrocyte sedimentation: Westergren's erythrocyte sedimentation over 28 mm/h.

4) In stage II˜III for joint function.

5) No active or remote peptic ulcer.

6) Without any serious parenchymatous disease on heart, liver, lung, kidney etc.

7) Without a history of drug allergy.

8) Not in pregnancy or period of child nursing.

9) Not administrated with, locally or generally, immunosuppressive drugs, D-penicillamine, chloroquine, gold compounds, and glucocorticoid hormones three weeks prior to the test.

Randomization and Statistical Exclusion

1) Patients in each of the site were randomized to two treatment groups: TGP and MTX groups. Conditions of patient were well balanced between groups to make it comparable in age, sex, duration of the disease, and severity of the symptoms.

2) Patient who quitted the treatment for the ineffectiveness 7 days after the start of administration was included in the efficacy analysis. Patient who quitted the treatment for the side effects was not included in the efficacy analysis but the safety analyses.

Procedures of the Study

Administration

1) The placebo run-in period: Seven days before administration, the selected patients stopped using NSAIDs and took a placebo instead. Symptoms would be aggravated during this time.

2) For the high dose study: Patients in the TGP group receive TGP capsules 0.6 g (two capsules) each time, 3 times a day. Those in MTX group took MTX tablet, 15 mg each time, once every week.

3) For the low dose study, patients in the TGP group received TGP capsules 0.6 g (two capsules) each time, 2 times a day. Those in the MTX group took MTX tablet, 7.5 mg each time, once every week.

4) For the double-blinded study, patients in both groups would be given capsules and tablets in same shapes, odors and amount. Placebo of capsule or tablet were included. Neither doctors nor patients can tell the group of treatment from the other.

5) No analgesics, anti-inflammation drugs, immune regulatory agents or muscle relaxants were allowed during the period of study. Acetaminophen could be used when necessary. If a physical therapy was started before enrollment, it was allowed to be continued, yet, it should be taken all the way during the period of clinical study.

Scoring for Symptoms and Physical Examination

1) Pain at rest: patients were asked to scale the degree of pain with a ruler

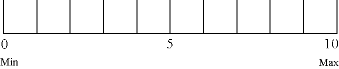

2) Morning stiffness: Patients were requested to make notes on duration of their morning joint stiffness, a time span from its appearance to recovery in minutes every morning. The doctors should evaluate their record sheets by questioning them. For example “Do you fell stiff in any of your joints? (today, these 2 days)” When get a “yes” in answer, then “when did you get up today?” and “when does the stiffness be gone?”

3) Swelling of joints: Take count of the index of joint swelling by summing up the degree number of each joint.

0=normal; 1=swelling soft tissues (synovium) without hydrarthrus; 2=swelling soft tissues (synovium) with hydrarthrus.

4) Tenderness of joints: Take count of the index of joint tenderness by summing up the degree number of each joint:

0=normal, no pain occurred at press and maximum passive movement

1=mild, patient complains a pain deep pressed on the edge of joint or ligament while no limit for passive movement.

2=medium, together with a knitted brows, patient complains a pain deep pressed on the joint or ligament. A slight restriction in passive movement.

3=severe, with a withdrawal, patient complains a pain when deep pressed. A high-grade restriction in passive movement.

5) Grip strength: Fold the arm belt of a sphygmomanometer twice and seal it in a small cloth bag suitable for a hand to hold. Aerates the belt in pressure of 20 mm Hg. Patients were asked to grip the bag with their not braced left and right hand, the grip strength is the average pressures in mm Hg for three times.

6) Time consumed for a 15 meters' walk (Seconds): Time started from a calm standing to the final of 15 meters. The average of three times.

7) General joint function (criteria of ARA):

Grade I: All joints can move properly.

Grade II: Restrictions in joint movement of medium severity. At least one joint is dysfunctional with no problem in daily work and life.

Grade III: Obvious restriction in joint movement. One can only deal with limited activity to maintain an essential daily life.

Grade IV: Confined in bed or chair.

8) Self-scoring and physician's grading on disease conditions: Both patients and doctors were required to submit their score sheet every time on follow-up survey. The scoring was made upon following criteria:

0=no obvious pain and swelling in joints

1=slight pain and swelling in joints, no obvious affection on patient's daily life.

2=pain of medium severity, affect the patient's daily life.

3=severe sore and pain in joints, obviously affect the patient's daily life and a bed rest is required.

9) Patient's general condition: condition on the physical strength, sleep and appetite.

1 = good; 2 = medium; 3 = bad

Laboratory Tests and X-Ray Examination

10) Routine blood and urine exam: Hemoglobin, platelet count, biochemical serum test and routine urine test

11) Blood Sedimentation (Norm. M: 15 mm/hr F: 20 mm/hr)

12) Serum Rheumatoid Factor (Method: Latex Agglutination Test, LAT): The maximum titer value were required for positive reaction patients. All centers use the same standard agents in one product batch.

13) C-reactive protein (quantitative method): All centers use the same standard agents in one product batch.

14) Immunoglobulin (Ig A, Ig G and Ig M): (Single Radial Immunodiffusion Test)

15) Immunocyte Potency: Lymphocyte Transformation Rate (PHA, ³H-Tda Method of Edosmosis) and IL-1 produced by mononuclear leukocyte in peripheral circulation

16) X-ray Examination: At the beginning and by the end of the clinical study, patients were to take X-ray examinations in their hands. The pictures should include the wrists. X-ray examinations in other part of the body were also required if necessary. The index for X-ray examination is as follows:

Stage I: normal or osteoporosis at ends of both sides.

Stage II: osteoporosis at ends of both sides, cystic or erosive damages beneath the articular cartilage can be seldom observed.

Stage III: obvious cystic or erosive damages beneath the articular cartilage, narrowed joint space or subluxation of joint.

Stage IV: Besides lesions of stage II and III, there is a fibrous or bony ankylosis.

Comprehensive Evaluation on the Effectiveness of Treatments

Evaluation on Benefic Effectiveness

Researchers are required to give their comprehensive evaluation on effectiveness of the treatments at the end of a complete study, or after the study was ceased for any reasons.

No effect: less than 30% improvement in symptoms and physical signs.

Improved: About 50% improvement in symptoms and physical signs (including general conditions in physical strength, sleep and appetite). Index of laboratory tests in blood sedimentation and items of immune system showed an improvement.

Obviously improved: more than 75% improved symptoms and physical signs of inflammation (including improvement in general conditions of physical strength, sleep and appetite) Index of laboratory tests in blood sedimentation and items of immune system turned back to normal or nearly normal.

Evaluation on Adverse-Effectiveness

1) Side-Effects' Scoring

The eneral condition of patients was evaluated on each visit and follow-up survey. Patients were inquired of symptoms and signs into a 26-item list of side effect. Once a possible side effect occurred, the relevant information as time, severity, frequency, duration, method used in release and its result were recorded by the doctor. Doctors were also asked to give their suggestion upon the relationship between those effects and the tested drug. A second disease occurred during the study was not counted in as a side effect. Index of overall side-effect is as below:

0=nothing abnormal

1=mild symptoms which does not interfere with a normal daily life

2=upset of medium severity, interfere with the daily life of patient

3=sever unwell, obviously affect a normal daily life and may need a bed rest.

4=life threatening side effect

2) Evaluation of Drug Toleration

Investigators should score individually on drug toleration in each patient after the test base on the following criteria:

0=bear sever side effect(s) and need to halt the test

1=side effects(s) of medium severity, relevant treatments is adopted.

2=mild side effect(s), no treatment was needed

3=no side effect.

Discontinuance of the Test

A detailed prescription on reasons of withdrawal should be recorded together with the date of withdrawal by the doctor responsible for the patient. The four possible reasons for withdrawal can be prescribed as following. (Test case should be replenished upon the first three conditions)

1) Case that was not in compliant with the criteria of patient selection found after the begging of the test. i.e. those whose symptoms were not getting worse during washout period.

2) For reasons other than side effects: happened to have other diseases, or move to other areas etc.

3) For poor effect of treatment, patient stop using drugs or giving up the test. Test period was less than a week.

4) Treatment have to be ceased for the sever side effect(s).

Test Results

TABLE 56
Effective rate of two groups (double blind, TGP n = 60, MTX n = 60)

| Period of | | No | Im- | Obviously | Effective | P |
|---|---|---|---|---|---|---|
| treatment | Group | effect | proved | improved | Rate | value |
| 4 weeks | TGP | 27 | 29 | 4 | 55.0% | 0.1569 |
| | | (45.0%) | (48.3%) | (6.7%) | | |
| | MTX | 34 | 19 | 7 | 43.3% | |
| | | (56.7%) | (31.7%) | (11.7%) | | |
| 8 weeks | TGP | 22 | 23 | 15 | 63.3% | 0.4780 |
| | | (36.7%) | (38.3%) | (25.0%) | | |
| | MTX | 20 | 19 | 21 | 66.7% | |
| | | (33.3%) | (31.7%) | (35.0%) | | |
| 12 weeks | TGP | 17 | 19 | 24 | 71.7% | 0.6525 |
| | | (28.3%) | (31.7%) | (40. 0%) | | |
| | MTX | 15 | 16 | 29 | 75.0% | |
| | | (25.0%) | (26.7%) | (48.33%) | | |

TABLE 57
Changes in RA perimeters: symptoms and physical
examinations (double blind, mean ± SD)

| Period of | | TGP | MTX | P value | | |
|---|---|---|---|---|---|---|
| treatment | Perimeters | (n = 60) | (n = 60) | TGP | MTh | P value |
| 4 weeks | PR | 1.53 ± | 1.33 ± | <0.0001 | <0.0001 | 0.5050 |
| | | 1.89 | 1.27 | | | |
| | MR | 30.29 | 33.52 ± | <0.0001 | <0.0001 | 0.7399 |
| | | 50.63 | 55.34 | | | |
| | GS-L | 25.27 | 12.70 ± | 0.0475 | <0.0001 | 0.6769 |
| | | 96.70 | 15.51 | | | |
| | GS-R | 9.20 ± | 12.05 ± | 0.0126 | <0.0001 | 0.5194 |
| | | 27.70 | 19.00 | | | |
| | NTJ | 2.81 ± | 3.53 ± | 0.0013 | <0.0001 | 0.5476 |
| | | 6.44 | 3.44 | | | |
| | ITJ | 5.16 ± | 6.83 ± | <0.0001 | <0.0001 | 0.1814 |
| | | 7.98 | 5.62 | | | |
| | NSJ | 1.41 ± | 1.92 ± | 0.0007 | <0.0001 | 0.6760 |
| | | 3.06 | 2.42 | | | |
| | ISJ | 2.21 ± | 2.47 ± | 0.0011 | <0.0001 | 0.7324 |
| | | 5.00 | 3.04 | | | |
| 8 weeks | PR | 1.97 ± | 1.94 ± | <0.0001 | <0.0001 | 0.9204 |
| | | 2.13 | 1.02 | | | |
| | MR | 42.24 ± | 41.03 ± | <0.0001 | <0.0001 | 0.9172 |
| | | 72.53 | 57.95 | | | |
| | GS-L | 17.18 ± | 17.55 ± | <0.0001 | <0.0001 | 0.9369 |
| | | 26.69 | 21.99 | | | |
| | GS-R | 13.55 ± | 16.68 ± | 0.0015 | <0.0001 | 0.5445 |
| | | 31.58 | 23.28 | | | |
| | NTJ | 3.59 ± | 5.52 ± | 0.0002 | <0.0001 | 0.0661 |
| | | 6.95 | 4.21 | | | |
| | ITJ | 7.47 ± | 9.53 ± | <0.0001 | <0.0001 | 0.1449 |
| | | 8.45 | 6.95 | | | |
| | NSJ | 1.98 ± | 2.97 ± | 0.0002 | <0.0001 | 0.1139 |
| | | 3.86 | 2.87 | | | |
| | ISJ | 3.33 ± | 4.03 ± | 0.0001 | <0.0001 | 0.5460 |
| | | 6.29 | 3.40 | | | |
| 12 weeks | PR | 0.48 | 2.60 ± | <0.0001 | <0.0001 | 0.7489 |
| | | 2.24 | 1.74 | | | |
| | MR | 54.02 ± | 46.08 ± | <0.0001 | <0.0001 | 0.5578 |
| | | 81.98 | 61.32 | | | |
| | GS, L | 22.73 ± | 22.03 ± | <0.0001 | <0.0001 | 0.8887 |
| | | 30.23 | 26.32 | | | |
| | GS, R | 21.09 ± | 21.85 ± | <0.0001 | <0.0001 | 0.8766 |
| | | 29.39 | 26.61 | | | |
| | NTJ | 5.66 ± | 6.73 ± | <0.0001 | <0.0001 | 0.6458 |
| | | 7.31 | 5.04 | | | |
| | ITJ | 9.12 ± | 11.59 ± | <0.0001 | <0.0001 | 0.1437 |
| | | 10.35 | 7.84 | | | |
| | NSJ | 2.91 ± | 3.92 ± | <0.0001 | <0.0001 | 0.123 |
| | | 3.52 | 3.57 | | | |
| | TSJ | 4.14 ± | 5.08 ± | <0.0001 | <0.0001 | 0.306 |
| | | 5.44 | 4.50 | | | |

TABLE 58
Changes in main perimeters scoring for the
effectiveness, double blinded (1)

| | | Cases in | | |
|---|---|---|---|---|
| | | TGP | | |
| | | group | | |
| | | (n = 60) | | |
| Items | Effectiveness | 4 weeks | 8 weeks | 12 weeks |
| MR | Disappeared | 11(18.3%) | 12(20.0%) | 15(25.0%) |
| | 50% reduced | 27(45.0%) | 29(48.3%) | 36(60.0%) |
| ITJ | Disappeared | 1(1.7%) | 3(5.0%) | 14(23.3%) |
| | 50% reduced | 17(28.3%) | 22(36.7%) | 22(36.7%) |
| NSJ | Disappeared | 8(13.3%) | 14(23.3%) | 15(31.7%) |
| | 50% reduced | 29(48.3%) | 28(46.7%) | 37(61.7%) |
| AGS | 50% | 3(5.0%) | 4(6.7%) | 9(15.0%) |
| | increased | | | |
| ESR | Reduced to | 18(30.0%) | 14(23.3%) | 19(31.7%) |
| | norm. | | | |
| | 50% reduced | 19(31.7%) | 29(48.3%) | 34(56.7%) |
| CRP | Reduced to | 7(11.7%) | 20(33.3%) | 19(31.7%) |
| | Norm. | | | |
| | 50% reduced | 16(26.7%) | 14(23.3%) | 16(26.6%) |
| RF | Reduced to | 0 | 0 | 0 |
| | norm. | | | |
| | 2 dilutions | 7(11.67%) | 20(33.3%) | 24(40.0%) |
| | lower | | | |

TABLE 59
Changes in main perimeters scoring for the
effectiveness, double blinded (2)

| | Cases an MTX | | |
|---|---|---|---|
| | group (n = 60) | | |
| Items | 4 weeks | 8 weeks | 12 weeks |
| MR | 15(25.0%) | 13(21.7%) | 15(25.0%) |
| | 23(38.3%) | 32(53.3%) | 36(60.0%) |
| ITJ | 2(3.3%) | 2(3.3%) | 9(15.0%) |
| | 19(31.7%) | 26(43.3%) | 31(51.7%) |
| NSJ | 9(15.0%) | 12(20.0%) | 16(26.7%) |
| | 24(40.0%) | 33(55.0%) | 34(56.7%) |
| AGS | 3(5.0%) | 6(10.0%) | 9(15.0%) |
| ESR | 16(26.7%) | 19(31.7%) | 20(33.3%) |
| | 18(30.0%) | 25(41.7%) | 31(51.7%) |
| CRP | 4(6.7%) | 15(25.0%) | 5(8.3%) |
| | 10(16.7%) | 16(26.7%) | 20(33.3%) |
| RF | 0 | 0 | 0 |
| | 8(13.3%) | 22(36.7%) | 22(36.7%) |

TABLE 60
Overall effectiveness ot the two treatment groups

| | | Number of patient | | | | |
|---|---|---|---|---|---|---|
| Period of | | | | Obv. | Effective | P |
| treatment | Groups | No effect | Improved | improved | rate | value |
| 4 weeks | TGP | 147(49.0%) | 116(38.7%) | 37(12.3%) | 51.0% | 0.7253 |
| | (n = 300) | | | | | |
| | MTX | 75(52.8%) | 52(36.6%) | 15(10.6%) | 47.2% | |
| | (n = 142) | | | | | |
| 8 weeks | TGP | 94(31.3%) | 99(33.0%) | 107(35.7%) | 68.7% | 0.7125 |
| | (n = 300) | | | | | |
| | MTX | 50(35.2%) | 45(31.7%) | 47(33.1%) | 64.8% | |
| | (n = 142) | | | | | |
| 12 weeks | TGP | 63(21.0%) | 69(23.0%) | 168(56.0) | 79.0% | 0.2970 |
| | (n = 300) | | | | | |
| | MTX | 32(22.5%) | 41(28.9%) | 69(48.6%) | 77.5% | |
| | (n = 142) | | | | | |
| 24 weeks | TGP | 13(20.6%) | 12(19.1%) | 38(60.3%) | 79.4% | 0.3007 |
| | (n = 63) | | | | | |
| | MTX | 11(18.3%) | 6(10.0%) | 43(71.7%) | 81.7% | |
| | (n = 60) | | | | | |

TABLE 61
Changes in RA perimeters of symptoms and physical examinations
(overall groups, mean ± SD)

| Period of | Peri- | TGP | MTX | P value | | |
|---|---|---|---|---|---|---|
| treatment | meters | (n = 300) | (n = 142) | TGP | NTX | P value |
| 4 weeks | PR | 1.66 ± | 1.05 ± | <0.0001 | <0.0001 | 0.0003 |
| | | 1.71 | 1.16 | | | |
| | MR | 27.98 ± | 35.76 ± | <0.0001 | <0.0001 | 0.0548 |
| | | 39.95 | 47.96 | | | |
| | GS-L | 12.33 ± | 12.56 ± | <0.0001 | <0.0001 | 0.9540 |
| | | 46.15 | 14.80 | | | |
| | GS-R | 9.14 ± | 11.60 ± | <0.0001 | <0.0001 | 0.1996 |
| | | 20.26 | 15.58 | | | |
| | NTJ | 3.29 ± | 2.61 ± | <0.0001 | <0.0001 | 0.1388 |
| | | 5.00 | 3.38 | | | |
| | ITJ | 6.79 ± | 6.04 ± | <0.0001 | <0.0001 | 0.6204 |
| | | 8.89 | 6.56 | | | |
| | NSJ | 2.45 ± | 2.10 ± | <0.0001 | <0.0001 | 0.6241 |
| | | 3.83 | 3.83 | | | |
| | ISJ | 3.96 ± | 3.73 ± | <0.0001 | <0.0001 | 0.7410 |
| | | 7.11 | 5.93 | | | |
| 8 weeks | PR | 2.37 ± | 1.94 ± | <0.0001 | <0.0001 | 0.0394 |
| | | 2.06 | 1.76 | | | |
| | MR | 36.93 ± | 54.20 ± | <0.0001 | <0.0001 | 0.0030 |
| | | 48.47 | 68.92 | | | |
| | GS-L | 16.58 ± | 16.66 ± | <0.0001 | 0.0043 | 0.7364 |
| | | 49.42 | 68.34 | | | |
| | GS-R | 13.93 ± | 19.36 ± | <0.0001 | <0.0001 | 0.0261 |
| | | 24.24 | 23.89 | | | |
| | NTJ | 5.72 ± | 5.38 ± | <0.0001 | <0.0001 | 0.6249 |
| | | 6.70 | 6.56 | | | |
| | ITJ | 12.02 ± | 12.66 ± | <0.0001 | <0.0001 | 0.6171 |
| | | 10.73 | 15.02 | | | |
| | NSJ | 4.44 ± | 4.43 ± | <0.0001 | <0.0001 | 0.6573 |
| | | 6.83 | 6.13 | | | |
| | TSJ | 7.17 ± | 6.59 ± | <0.0001 | <0.0001 | 0.5019 |
| | | 7.81 | 9.11 | | | |
| 12 weeks | PR | 3.09 ± | 3.36 ± | <0.0001 | <0.0001 | 0.2738 |
| | | 2.38 | 2.40 | | | |
| | MR | 55.83 ± | 78.52 ± | <0.0001 | <0.0001 | 0.0014 |
| | | 57.31 | 84.81 | | | |
| | GS-L | 20.99 ± | 23.06 ± | <0.0001 | 0.0002 | 0.6547 |
| | | 27.10 | 72.27 | | | |
| | GS-R | 21.23 ± | 28.10 ± | <0.0001 | <0.0001 | 0.0018 |
| | | 25.67 | 32.01 | | | |
| | NTJ | 8.06 ± | 8.78 ± | <0.0001 | <0.0001 | 0.6090 |
| | | 7.84 | 8.48 | | | |
| | ITJ | 15.75 ± | 18.77 ± | <0.0001 | <0.0001 | 0.0655 |
| | | 13.65 | 20.63 | | | |
| | NSJ | 5.83 ± | 6.77 ± | <0.0001 | <0.0001 | 0.2956 |
| | | 8.99 | 8.23 | | | |
| | ISJ | 9.12 | 10.25 ± | <0.0001 | <0.0001 | 0.2575 |
| | | 8.57 | 12.09 | | | |
| n = 63 | | | | n = 60 | | |
| 24 weeks | PR | 2.13 ± | 2.62 ± | <0.0001 | <0.0001 | 0.2423 |
| | | 2.34 | 2.11 | | | |
| | MR | 59.69 ± | 60.70 ± | <0.0001 | <0.0001 | 0.2055 |
| | | 70.08 | 63.51 | | | |
| | GS-L | 31.75 ± | 31.83 ± | <0.0001 | <0.0001 | 0.9558 |
| | | 33.18 | 25.89 | | | |
| | GS-R | 32.13 ± | 30.90 ± | <0.0001 | <0.0001 | 0.1270 |
| | | 26.08 | 26.29 | | | |
| | NTJ | 11.05 ± | 9.05 ± | <0.0001 | <0.0001 | 0.2099 |
| | | 9.09 | 7.73 | | | |
| | ITJ | 11.35 ± | 13.94 ± | <0.0001 | <0.0001 | 0.2199 |
| | | 11.88 | 10.31 | | | |
| | NSJ | 5.68 ± | 5.39 ± | <0.0001 | <0.0001 | 0.7430 |
| | | 4.55 | 4.94 | | | |
| | ISJ | 6.98 ± | 6.88 ± | <0.0001 | <0.0001 | 0.9129 |
| | | 5.23 | 5.68 | | | |

TABLE 62
Changes in main perimeters scoring for
effectiveness (overall group)

| | | Cases in TGP group | | | |
|---|---|---|---|---|---|
| | | (n = 300 for studies within 12 weeks; | | | |
| | | n = 63 for study in 24 weeks) | | | |
| Items | Effectiveness | 4 weeks | 8 weeks | 12 weeks | 24 weeks |
| MR | Disappeared | 29(9.7%) | 33(11.0%) | 42(14.0%) | 23(36.5%) |
| | 50% reduced | 135(45.0%) | 162(54.0%) | 201(67.0%) | 27(42.9%) |
| TTJ | Disappeared | 3(1.0%) | 9(3.0%) | 16(5.3%) | 5(7.9%) |
| | 50% reduced | 64(21.3%) | 88(29.3%) | 170(36.7%) | 40(63.5%) |
| NSJ | Disappeared | 27(9.0%) | 39(13.0%) | 50(16.7%) | 14(22.2%) |
| | 50% reduced | 138(46.0%) | 173(57.7%) | 194(64.7%) | 34(54.0%) |
| AGS | 50% increased | 41(13.7%) | 59(19.7%) | 92(30.7%) | 12(19.1%) |
| ESR | Reduced to | 43(14.3%) | 75(25.0%) | 84(28.0%) | 24(38.1%) |
| | norm. | | | | |
| | 50% reduced | 88(29.3%) | 134(44.7%) | 143(47.7%) | 18(28.6%) |
| CRP | Reduced to | 9(3.0%) | 24(8.0%) | 53(17.7%) | 24(38.1%) |
| | Norm. | | | | |
| | 50% reduced | 64(21.3%) | 39(13.0%) | 42(14.0%) | 13(20.6%) |
| RF | Reduced to | 6(2.0%) | 35(11.7%) | 28(9.3%) | 6(9.5%) |
| | norm. | | | | |
| | 2 dilutions | 54(18.0%) | 120(40.0%) | 152(50.7%) | 11(17.5%) |
| | lower | | | | |

TABLE 63
Changes in main perimeters scoring for
effectiveness (overall group)

| | | Cases in MTX group | | | |
|---|---|---|---|---|---|
| | | (n = 150 for studies within 12 weeks; | | | |
| | | n = 60 for study an 24 weeks) | | | |
| Items | Effectiveness | 4 weeks | 8 weeks | 12 weeks | 24 weeks |
| MR | Disappeared | 14(9.8%) | 28(19.7%) | 34(11.3%) | 19(31.7%) |
| | 50% | 65(45.8%) | 74(52.1%) | 96(67.6%) | 30(50.0%) |
| | reduced | | | | |
| ITJ | Disappeared | 4(2.8%) | 5(3.5%) | 9(6.3%) | 6(10.0%) |
| | 50% | 27(19.0%) | 49(34.5%) | 72(50.7%) | 35(58.3%) |
| | reduced | | | | |
| NSJ | Disappeared | 15(10.6%) | 24(16.9%) | 27(19.0%) | 16(26.7%) |
| | 50% | 69(48.6%) | 73(51.4%) | 87(61.3%) | 28(46.7%) |
| | reduced | | | | |
| AGS | 50% | 19(7.5%) | 23(16.7%) | 42(29.5%) | 10(16.7%) |
| | increased | | | | |
| ESR | Reduced to | 22(15.5%) | 26(18.3%) | 31(21.8%) | 26(43.3%) |
| | norm. | | | | |
| | 50% | 59(41.6%) | 80(56.3%) | 76(53.5%) | 14(23.3%) |
| | reduced | | | | |
| CRP | Reduced to | 7(4.9%) | 18(12.7%) | 25(17.6%) | 24(40.0%) |
| | Norm. | | | | |
| | 50% | 19(13.4%) | 25(17.6%) | 33(23.2%) | 11(18.3%) |
| | reduced | | | | |
| RF | Reduced to | 7(4.9%) | 13(9.2%) | 16(11.3%) | 6(10.0%) |
| | norm. | | | | |
| | 2 | 70(49.3%) | 85(59.9%) | 85(59.9%) | 23(38.3%) |
| | dilutions | | | | |
| | lower | | | | |

TABLE 64
Changes in tests on functional immune system

| Period | | | | |
|---|---|---|---|---|
| of | TGP (n = 60, n = 20 for 24-week study) | | MTX (n-30, n = 10 for 24-week study) | |
| treatment | PHA | IL-1 | PHA | IL-1 |
| 0 | 16.42 ± 3.23 | 29.86 ± 4.56 | 17.03 ± 3.80 | 27.93 ± 4.57 |
| 4 weeks | 19.45 ± 3.53* | 21.17 ± 3.72* | 17.42 ± 4.00 | 26.42 ± 4.03 |
| 8 weeks | 21.21 ± 3.27* | 18.95 ± 3.95* | 17.85 ± 3.43 | 27.38 ± 4.34 |
| 12 weeks | 22.64 ± 3.26** | 17.84 ± 3.72** | 17.47 ± 3.16 | 27.29 ± 4.34 |
| 24 weeks | 25.46 ± 3.53** | 16.32 ± 3.15** | 17.68 ± 3.27 | 26.86 ± 3.36 |

TABLE 65
Changes in perimeters of immune system (1)

| | Period of | | |
|---|---|---|---|
| Group | treatment | ESR | CRP |
| TCP | 0 | 45.13 ± 23.29 | 29.52 ± 21.42 |
| n = 300 | 4 weeks | 39.05 ± 29.91 | 27.28 ± 21.38 |
| (n = 63 for 24- | 8 weeks | 32.9 ± 19.23* | 22.89 ± 19.71 |
| week test) | 12 weeks | 29.96 ± 19.96* | 22.42 ± 20.17 |
| | 24 weeks | 26.42 ± 23.96* | 18.09 ± 11.55* |
| MTX | 0 | 47.11 ± 26.19 | 32.01 ± 22.75 |
| n = 142 | 4 weeks | 42.11 ± 38.68 | 27.97 ± 21.12 |
| (n = 60 for 24- | 8 weeks | 44.92 ± 78.36 | 25.72 ± 19.42 |
| week test) | 12 weeks | 29.96 ± 20.48* | 22.31 ± 18.91 |
| | 24 weeks | 28.66 ± 20.96* | 27.12 ± 22.39 |

TABLE 66
Changes in perimeters of immune system (2)

| Group | RF | Ig A | Ig G | Ig M |
|---|---|---|---|---|
| TCP | 1.87 ± 0.59 | 3.25 ± 2.19 | 20.48 ± 9.59 | 2.34 ± 1.68 |
| n = 300 | 1.68 ± 0.57 | 3.07 ± 1.98 | 18.76 ± 8.26 | 2.20 ± 1.36 |
| (n = 63 | 1.51 ± 0.55 | 3.01 ± 1.72 | 18.02 ± 7.15 | 2.08 ± 1.15 |
| for 24- | 1.56 ± 0.55 | 2.79 ± 1.72 | 17.03 ± 6.27 | 1.82 ± 0.87 |
| week test) | 1.42 ± 0.53 | 2.82 ± 2.00 | 17.95 ± 7.47 | 1.32 ± 1.43 |
| MTX | 1.93 ± 0.61 | 3.62 ± 3.42 | 18.13 ± 7.12 | 2.45 ± 1.89 |
| n = 142 | 1.87 ± 0.62 | 3.64 ± 3.54 | 17.40 ± 6.59 | 2.62 ± 2.15 |
| (n = 60 | 1.59 ± 0.61 | 3.98 ± 3.56 | 17.62 ± 6.37 | 2.76 ± 2.24 |
| for 24- | 1.72 ± 0.56 | 3.39 ± 3.15 | 16.47 ± 5.92 | 2.28 ± 1.84 |
| week test) | 1.74 ± 0.63 | 2.35 ± 1.55 | 15.26 ± 6.37 | 1.57 ± 0.79 |

TABLE 67
Laboratory blood tests, Overall group (1)

| Group | Period of treatment | Hg | WBC |
|---|---|---|---|
| TCP | 0 | 10.86 ± 3.25 | 5.40 ± 1.80 |
| n = 300 | 4 weeks | 10.64 ± 3.21 | 5.76 ± 1.56 |
| (n = 63 for | 8 weeks | 10.23 ± 3.09 | 6.12 ± 2.04 |
| 24-week | 12 weeks | 10.97 ± 2.92 | 6.33 ± 2.11 |
| test) | 24 weeks | 11.00 ± 1.76 | 5.82 ± 1.80 |
| | P value | >0.05 | >0.05 |
| MTX | 0 | 11.28 ± 3.72 | 7.96 ± 2.61 |
| n = 142 | 4 weeks | 10.96 ± 3.65 | 7.47 ± 2.09 |
| (n = 60 for | 8 weeks | 9.26 ± 3.09 | 8.47 ± 2.82 |
| 24-week | 12 weeks | 9.24 ± 2.98 | 8.42 ± 2.57 |
| test) | 24 weeks | 10.78 ± 3.10 | 7.28 ± 2.67 |
| | P value | >0.05 | >0.05 |

TABLE 68
Laboratory blood tests, Overall group (2)

| Group | BP | Cr | BUN | GPT |
|---|---|---|---|---|
| TGP | 168.00 ± 56.00 | 78.96 ± 26.32 | 4.82 ± 1.61 | Norm. |
| n = 300 | 142.67 ± 47.58 | 73.21 ± 24.72 | 4.25 ± 1.42 | Norm. |
| (n = 63 | 128.31 ± 42.69 | 62.59 ± 20.86 | 3.97 ± 1.32 | Norm. |
| for | 158.64 ± 52.57 | 59.63 ± 19.96 | 3.82 ± 1.28 | Norm. |
| 24-week | 138.40 ± 44.26 | 68.94 ± 22.00 | 4.12 ± 1.72 | Norm. |
| test) | >0.05 | >0.05 | >0.05 | |
| MTX | 98.97 ± 32.86 | 74.86 ± 28.28 | 3.96 ± 1.32 | Norm. |
| n = 142 | 94.28 ± 31.42 | 82.82 ± 27.64 | 3.32 ± 1.11 | Norm. |
| (n = 60 | 108.73 ± 36.21 | 98.89 ± 32.93 | 1.97 ± 0.65 | Norm. |
| for | 110.66 ± 37.48 | 102.03 ± 36.86 | 1.82 ± 0.47 | Norm. |
| 24-week | 168.48 ± 30.78 | 78.66 ± 26.42 | 3.38 ± 1.01 | Norm. |
| test) | >0.05 | >0.05 | >0.05 | |

TABLE 69
Events of reverse drug effects in the two groups (double-blind)

| | TGP (n = 60) | | | | | | MTX (n = 60) | | | | | |
|---|---|---|---|---|---|---|---|---|---|---|---|---|
| Reverse | 4 weeks | | | 12 weeks | | | 4 weeks | | | 12 weeks | | |
| effect | 1 | 2 | 3 | 1 | 2 | 3 | 1 | 2 | 3 | 1 | 2 | 3 |
| Headache | | | | | | | 1 | | | 1 | | |
| Dizziness | | | | | | | 3 | | | 1 | | |
| Insomnia | | | | | | | | | | | 1 | |
| Lethargy | | | | | | | | | | 1 | | |
| Nausea | | | | 2 | | | | | | 4 | 4 | |
| Bad | | | | 2 | | | | | | 2 | 2 | |
| appetite | | | | | | | | | | | | |
| Abdo- | | | | 2 | | | | | | | | |
| distension | | | | | | | | | | | | |
| Frequency | | | | | | | | | | 1 | | |
| of | | | | | | | | | | | | |
| micturition | | | | | | | | | | | | |

TABLE 70
Events of reverse drug effects in the two groups (overall group)

| | TGP | | | | | | | | | | | | MTX | | | | | | | | | | | |
|---|---|---|---|---|---|---|---|---|---|---|---|---|---|---|---|---|---|---|---|---|---|---|---|---|
| | 4 weeks | | | 8 weeks | | | 12 weeks | | | 24 weeks | | | 4 weeks | | | 8 weeks | | | 12 weeks | | | 24 weeks | | |
| | (n = 300) | | | (n = 266) | | | (n = 300) | | | (n = 63) | | | (n = 150) | | | (n = 120) | | | (n = 300) | | | (n = 60) | | |
| Side effects | 1 | 2 | 3 | 1 | 2 | 3 | 1 | 2 | 3 | 1 | 2 | 3 | 1 | 2 | 3 | 1 | 2 | 3 | 1 | 2 | 3 | 1 | 2 | 3 |
| Headache | 4 | | | 1 | | | 1 | | | | | | 1 | 1 | | 2 | 1 | | 2 | 1 | | 2 | | |
| Dizziness | 4 | | | 3 | | | 3 | | | | | | 6 | | | 4 | | | 4 | | | 1 | | |
| Insomnia | 5 | 2 | | 5 | | 1 | 3 | 1 | | | | | | | | 5 | | | 5 | 1 | | 1 | 1 | |
| Lethargy | 1 | | | 1 | | | 1 | | | | | | 1 | | | 2 | | | 1 | | | | | |
| Palpitation | 7 | | | 3 | | | 2 | | | | | | 3 | | | 3 | | | 2 | | | | | |
| Nausea | 6 | | | 5 | | | 5 | | | | | | 14 | 8 | | 16 | 7 | | 25 | 10 | | 7 | 2 | |
| Vomiting | 1 | | | 2 | | | 1 | 2 | | | | | 3 | | | 2 | 1 | | 3 | | | 2 | 1 | |
| Bad appetite | 11 | 2 | | 9 | 1 | | 8 | 1 | | | | | 27 | 3 | | 28 | 5 | 1 | 35 | 3 | | 3 | | |
| Stomachache | 1 | | | | | | | | | | | | 1 | 1 | 1 | | | | | | | | | |
| Burning heart | 2 | 1 | | 4 | | | 4 | | | | | | 1 | | 2 | 3 | 1 | 1 | 2 | | 1 | | | |
| Abd | 9 | | | 8 | | | 5 | | | | | | 7 | 5 | | 11 | 4 | 1 | 15 | 4 | | | | |
| distension | | | | | | | | | | | | | | | | | | | | | | | | |
| Constipation | 7 | 1 | | 6 | | 1 | 7 | | | | | | 3 | | | 3 | | 1 | 2 | | | | | |
| Diarrhea | 7 | | | 4 | | 1 | 4 | | | | | | 1 | 1 | | 1 | | | 1 | | | | | |
| Skin rash | | | | 1 | | | 1 | | | | | | | | | | 1 | | 1 | | | | | |
| Stomatitis | | | | | | | | | | | | | 2 | | | 9 | 1 | | 9 | 1 | | | | |

### REFERENCES

1. Medical Sciences Bulletin, published by Pharmaceutical information Associates, Ltd. http://pharminfo.com/pubs/msb/rheumart.html

2. Rheumatoid Arthritis Fact Sheet. Arthritis Foundation, American College of Rheumatology, www.rheumatology.org, www.arthritis.org

3. Medical Sciences Bulletin, published by Pharmaceutical information Associates, Ltd., Rheumatoid Arthritis Fact Sheet. Arthritis Foundation, American College of Rheumatology, www.medicinenet.com

4. Medical Sciences Bulletin, published by Pharmaceutical information Associates, Ltd., Rheumatoid Arthritis Fact Sheet. Arthritis Foundation, American College of Rheumatology, www.medicinenet.com

5. The Merck Manual of Diagnosis and Therapy, Section 5. Musculoskeketal and Connective Tissue Disorders. www.merk.com/pubs/mmanual/section5/chapter50/50a.htm

6. PDR for Herbal Medicine 1^{st} edition (1999): 517, 1008 Peonies. Timber Press, Oregon. Stern, Frederick C. (1946) <<Chinese medical herbs>> P. 646, Administrative Office

7. <<Chinese medical herbs>> P. 646, Administrative Office of TCM, China

8. Jin, Q. Quan, Pharmacological experimental methodology, Edited by Xu, Shuyun. People's health press. 1982:400-410

9. Sun, Ruiyuan, Pharmacological experimental methodology, Edited by Xu, Shuyun. People's health press. 1982:400-410

10. Regulation for New Drug Application. Administration of health and sanitation, P.R. China. 1985:33

11. Xu, shuyun et al. (Eds.) (1982) Pharmacological experimental methodology. People's health press. P 400

12. Members of compile group, Pharmacological Experiments, First edition. People's Health Press, Beijing. 1985:238

13. Chen, Z. M. Organic Chemistry, Edited by Xu, J. D. People's Health Press, 1984: 114

14. Schmid. W. The Micronucleus Test Mutation Res, 1975, 31:9

15. Heddle, J. A, et al. The induction of micronuclei as a measure of genetoxycity. Mutation Res, 1983, 123-1: 61

What is claimed is:

1. A composition comprising Paeoniflorin, Albiflorin, Oxypaeoniflorin, and Benzoylpaeomiflorin.

2. The composition of claim 1 wherein the proportion of Paeoniflorin, Albiflorin, Oxypaeoniflorin, and Benzoylpaeomiflorin is 85-90%.

3. The composition of claim 1 wherein the proportion of Paeoniflorin is no less than 35%.

4. The composition of claim 1, derived from an extract of white peony.

5. The pharmaceutical composition comprising an effective amount of Paeoniflorin, Albiflorin, Oxypaeoniflorin, and Benzoylpaeomiflorin and a pharmaceutically acceptable carrier.

6. The pharmaceutical composition of claim 5 wherein the proportion of Paeoniflorin, Albiflorin, Oxypaeoniflorin, and Benzoylpaeomiflorin is 85-90%.

7. The pharmaceutical composition of claim 5 wherein the proportion of Paeoniflorin is no less than 35%.

8. A formulation comprising the pharmaceutical composition of claim 4, wherein the formulation is a pill, capsule, granule, tablet, suspension, injection, syrup, or tincture.

9. A method for producing the composition of claim 1 comprising steps of:
a. obtaining appropriate herbal materials;
b. chopping the obtained herbal materials into small pieces;
c. immersing the herbal materials into an organic solvent for extraction.
d. separating the extracted materials into residue and solution and repeat step c appropriate times for the residue;
e. combining solutions from the extractions;
f. concentrating the solution from step e;
g. diluting the solution from step f to approximately 6.0 in pH;
h. extracting solutions from after step g in appropriate solution to obtain lipo-solutions;
i. concentrating the combined lipo-solution from step h; and
j. vacuum drying the extract from step (i) to obtain a composition of claim 1.

10. The method of claim 9, wherein in step c and d, the chopped herbs are extracted three times in 95% alcohol.

11. The method of claim 9, wherein in step h, solutions from after step g are extracted three times in ethyl acetate.

12. The method of claim 9, herb is white peony.

13. The composition produced by method of claim 9, 10, 11, or 12.

14. The composition of claim 13, comprising Paeoniflorin.

15. The composition of claim 14, further comprising Albiflorin, Oxypaeoniflorin, and Benzoylpaeomiflorin.

16. A pharmaceutical composition of claim 13 and a pharmaceutically acceptable carrier.

17. A method for treating arthritis in a subject comprising administering to the subject the pharmaceutical composition of claim 5 or claim 6.

18. A method for alleviating clinical symptoms in a subject suffering from arthritis comprising administering to the subject the pharmaceutical composition of claim 5 or 6.

19. The method of claim 17 or 18, wherein the arthritis is a rheumatic arthritis.

20. A method for adjusting immunity in a subject comprising administering to the subject the pharmaceutical composition of claim 1.

21. A compound with the structure set forth in FIG. 37.

22. A derivative of the compound of claim 21.

23. A composition comprising the compound of claim 21 or 22.

24. A pharmaceutical composition comprising the compound of claim 21 or 22 and a pharmaceutically acceptable carrier.

25. A method for treating inflammatory conditions or immune disorders in a subject comprising administering to the subject the pharmaceutical composition of claim 23 or claim 21.

26. A method for producing compound of claim 21 as set forth in FIG. 39.

27. Total Glycosides of Paeony as characterized by at least 4 of the 8 peaks recited, wherein if the retention time of paeoniflorin as 1, the corresponding value for relative retention times are: 0.73 for peak 1, 0.91 for peak 2, 1 for peak 3, 1.12 for peak 4, 1.29 for peak 5, 1.37 for peak 6, 1.54 for peak 7, and 2.16 for peak 8.

28. The fingerprinting of Total Glycosides of Paeony as set forth in FIG. 48.

29. Total Glycosides of Paeony of claim 27, characterized by at least 5 peak of the 8 peaks recited in claim 28.

30. Total Glycosides of Paeony of claim 27, characterized by at least 6 peak of the 8 peaks recited in claim 28.

31. Total Glycosides of Paeony of claim 27, characterized by at least 7 peak of the 8 peaks recited in claim 28.
